SEQUENCE LISTING (37 sequences)

SEQ ID NO: 1 - protein, 1672 aa, Artificial Sequence [sequence omitted]

SEQ ID NO: 2 - protein, 601 aa, Artificial Sequence
  Description of Artificial Sequence Synthetic polypeptide
KSSPYQKKTENPCAQRCLQSCQQEPDDLKQKACESRCTKLEYDPRCVYDPRGHTGTTNQRSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 3 - protein, 151 aa, Artificial Sequence
  Description of Artificial Sequence Synthetic polypeptide
RQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 4 - protein, 492 aa, Artificial Sequence
  Description of Artificial Sequence Synthetic polypeptide
VTFRQGGEENECQFQRLNAQRPDNRIESEGGYIETWNPNNQEFQCAGVALSRTVLRRNALRRPFYSNAPLEIYVQQGSGYFGLIFPGCPSTYEEPAQEGRRYQSQKPSRRFQVGQDDPSQQQQDSHQKVHRFDEGDLIAVPTGVAFWMYNDEDTDVVTVTLSDTSSIHNQLDQFPRRFYLAGNQEQEFLRYQQQQGSRPHYRQISPRVRGDEQENEGSNIFSGFAQEFLQHAFQVDRQTVENLRGENEREEQGAIVTVKGGLRILSPDEEDESSRSPPNRREEFDEDRSRPQQRGKYDENRRGYKNGIEETICSASVKKNLGRSSNPDIYNPQAGSLRSVNELDLPILGWLGLSAQHGTIYRNAMFVPHYTLNAHTIVVALNGRAHVQVVDSNGNRVYDEELQEGHVLVVPQNFAVAAKAQSENYEYLAFKTDSRPSIANQAGENSIIDNLPEEVVANSYRLPREQARQLKNNNPFKFFVPPFDHQSMREVA

SEQ ID NO: 5 - protein, 306 aa, Artificial Sequence
  Description of Artificial Sequence Synthetic polypeptide
VTFRQGGEENECQFQRLNAQRPDNRIESEGGYIETWNPNNQEFQCAGVALSRTVLRRNALRRPFYSNAPLEIYVQQGSGYFGLIFPGCPSTYEEPAQEGRRYQSQKPSRRFQVGQDDPSQQQQDSHQKVHRFDEGDLIAVPTGVAFWMYNDEDTDVVTVTLSDTSSIHNQLDQFPRRFYLAGNQEQEFLRYQQQQGSRPHYRQISPRVRGDEQENEGSNIFSGFAQEFLQHAFQVDRQTVENLRGENEREEQGAIVTVKGGLRILSPDEEDESSRSPPNRREEFDEDRSRPQQRGKYDENRRGYKN

SEQ ID NO: 6 - protein, 726 aa, Artificial Sequence
  Description of Artificial Sequence Synthetic polypeptide
MAPRSARRPLLLLLLLLLLGLMHCASAAMFMVKNGNGTACIMANFSAAFSVNYDTKSGPKNMTLDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENSLEVTFRQGGEENECQFQRLNAQRPDNRIESEGGYIETWNPNNQEFQCAGVALSRTVLRRNALRRPFYSNAPLEIYVQQGSGYFGLIFPGCPSTYEEPAQEGRRYQSQKPSRRFQVGQDDPSQQQQDSHQKVHRFDEGDLIAVPTGVAFWMYNDEDTDVVTVTLSDTSSIHNQLDQFPRRFYLAGNQEQEFLRYQQQQGSRPHYRQISPRVRGDEQENEGSNIFSGFAQEFLQHAFQVDRQTVENLRGENEREEQGAIVTVKGGLRILSPDEEDESSRSPPNRREEFDEDRSRPQQRGKYDENRRGYKNEFTLIPIAVGGALAGLVLIVLIAYLVGRKRSHAGYQTI

SEQ ID NO: 7 - protein, 571 aa, Artificial Sequence
  Description of Artificial Sequence Synthetic polypeptide
MAPRSARRPLLLLLLLLLLGLMHCASAAMFMVKNGNGTACIMANFSAAFSVNYDTKSGPKNMTLDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENSLERQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRYEFTLIPIAVGGALAGLVLIVLIAYLVGRKRSHAGYQTI

SEQ ID NO: 8 - protein, 1021 aa, Artificial Sequence
  Description of Artificial Sequence Synthetic polypeptide
MAPRSARRPLLLLLLLLLLGLMHCASAAMFMVKNGNGTACIMANFSAAFSVNYDTKSGPKNMTLDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENSLEKSSPYQKKTENPCAQRCLQSCQQEPDDLKQKACESRCTKLEYDPRCVYDPRGHTGTTNQRSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFNEFTLIPIAVGGALAGLVLIVLIAYLVGRKRSHAGYQTI

SEQ ID NO: 9 - protein, 186 aa, Artificial Sequence
  Description of Artificial Sequence Synthetic polypeptide
GIEETICSASVKKNLGRSSNPDIYNPQAGSLRSVNELDLPILGWLGLSAQHGTIYRNAMFVPHYTLNAHTIVVALNGRAHVQVVDSNGNRVYDEELQEGHVLVVPQNFAVAAKAQSENYEYLAFKTDSRPSIANQAGENSIIDNLPEEVVANSYRLPREQARQLKNNNPFKFFVPPFDHQSMREVA

SEQ ID NO: 10 - DNA, 1227 bp, Artificial Sequence
  Description of Artificial Sequence Synthetic polynucleotide
atggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagttcgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactggatgcccccaagttcttatatgtgcaacaaagagcagactgtttcagtgtctggagcatttcagataaatacctttgatctaagggttcagcctttcaatgtgacacaaggaaagtattctacagctcaagactgcagtgcagatgacgacaacttccttgtgcccatagcggtgggagctgccttggcaggagtacttattctagtgttgctggcttattttattggtctcaagcaccatcatgctggatatgagcaattttag

SEQ ID NO: 11 - DNA, 1248 bp, Artificial Sequence
  Description of Artificial Sequence Synthetic polynucleotide
atgccccggcagctcagcgcggcggccgcgctcttcgcgtccctggccgtaattttgcacgatggcagtcaaatgagagcaaaagcatttccagaaaccagagattattctcaacctactgcagcagcaacagtacaggacataaaaaaacctgtccagcaaccagctaagcaagcacctcaccaaactttagcagcaagattcatggatggtcatatcacctttcaaacagcggccacagtaaaaattccaacaactaccccagcgactacaaaaaacactgcaaccaccagcccaattacctacaccctggtcacaacccaggccacacccaacaactcacacacagctcctccagttactgaagttacagtcggccctagcttagccccttattcactgccacccaccatcaccccaccagctcatacaactggaaccagttcatcaaccgtcagccacacaactgggaacaccactcaacccagtaaccagaccacccttccagcaactttatcgatagcactgcacaaaagcacaaccggtcagaagcctgttcaacccacccatgccccaggaacaacggcagctgcccacaataccacccgcacagctgcacctgcctccacggttcctgggcccacccttgcacctcagccatcgtcagtcaagactggaatttatcaggttctaaacggaagcagactctgtataaaagcagagatggggatacagctgattgttcaagacaaggagtcggttttttcacctcggagatacttcaacatcgaccccaacgcaacgcaagcctctgggaactgtggcacccgaaaatccaaccttctgttgaattttcagggcggatttgtgaatctcacatttaccaaggatgaagaatcatattatatcagtgaagtgggagcctatttgaccgtctcagatccagagacaatttaccaaggaatcaaacatgcggtggtgatgttccagacagcagtcgggcattccttcaagtgcgtgagtgaacagagcctccagttgtcagcccacctgcaggtgaaaacaaccgatgtccaacttcaagcctttgattttgaagatgaccactttggaaatgtggatgagtgctcgtctgactacacaattgtgcttcctgtgattggggccatcgtggttggtctctgccttatgggtatgggtgtctataaaatccgcctaaggtgtcaatcatctggataccagagaatc

SEQ ID NO: 12 - DNA, 594 bp, Artificial Sequence
  Description of Artificial Sequence Synthetic polynucleotide
atgtcgcggctctcccgctcactgctttgggccgccacctgcctgggcgtgctctgcgtgctgtccgcggacaagaacacgacccagcacccgaacgtgacgactttagcgcccatctccaacgtaacctcggcgccggtgacgtccctcccgctggtcaccactccggcaccagaaacctgtgaaggtcgaaacagctgcgtttcctgttttaatgttagcgttgttaatactacctgcttttggatagaatgtaaagatgagagctattgttcacataactcaacagttagtgattgtcaagtggggaacacgacagacttctgttccgtttccacggccactccagtgccaacagccaattctacagctaaacccacagttcagccctccccttctacaacttccaagacagttactacatcaggtacaacaaataacactgtgactccaacctcacaacctgtgcgaaagtctacctttgatgcagccagtttcattggaggaattgtcctggtcttgggtgtgcaggctgtaattttctttctttataaattctgcaaatctaaagaacgaaattaccacactctgtaa

SEQ ID NO: 13 - DNA, 1437 bp, Artificial Sequence
  Description of Artificial Sequence Synthetic polynucleotide
atgggccgatgctgcttctacacggcggggacgttgtccctgctcctgctggtgaccagcgtcacgctgctggtggcccgggtcttccagaaggctgtagaccagagtatcgagaagaaaattgtgttaaggaatggtactgaggcatttgactcctgggagaagccccctctgcctgtgtatactcagttctatttcttcaatgtcaccaatccagaggagatcctcagaggggagacccctcgggtggaagaagtggggccatacacctacagggaactcagaaacaaagcaaatattcaatttggagataatggaacaacaatatctgctgttagcaacaaggcctatgtttttgaacgagaccaatctgttggagaccctaaaattgacttaattagaacattaaatattcctgtattgactgtcatagagtggtcccaggtgcacttcctcagggagatcatcgaggccatgttgaaagcctatcagcagaagctctttgtgactcacacagttgacgaattgctctggggctacaaagatgaaatcttgtcccttatccatgttttcaggcccgatatctctccctattttggcctattctatgagaaaaatgggactaatgatggagactatgtttttctaactggagaagacagttaccttaactttacaaaaattgtggaatggaatgggaaaacgtcacttgactggtggataacagacaagtgcaatatgattaatggaacagatggagattcttttcacccactaataaccaaagatgaggtcctttatgtcttcccatctgacttttgcaggtcagtgtatattactttcagtgactatgagagtgtacagggactgcctgcctttcggtataaagttcctgcagaaatattagccaatacgtcagacaatgccggcttctgtatacctgagggaaactgcctgggctcaggagttctgaatgtcagcatctgcaagaatggtgcacccatcattatgtctttcccacacttttaccaagcagatgagaggtttgtttctgccatagaaggcatgcacccaaatcaggaagaccatgagacatttgtggacattaatcctttgactggaataatcctaaaagcagccaagaggttccaaatcaacatttatgtcaaaaaattagatgactttgttgaaacgggagacattagaaccatggttttcccagtgatgtacctcaatgagagtgttcacattgataaagagacggcgagtcgactgaagtctatgattaacactactttgatcatcaccaacataccctacatcatcatggcgctgggtgtgttctttggtttggtttttacctggcttgcatgcaaaggacagggatccatggatgagggaacagcggatgaaagagcacccctcattcgaacctag

SEQ ID NO: 14 - DNA, 3756 bp, Artificial Sequence
  Description of Artificial Sequence Synthetic polynucleotide
aagtccagcccctaccagaagaaaaccgagaacccctgcgcccagcggtgcctgcagtcttgtcagcaggaacccgacgacctgaagcagaaggcctgcgagagccggtgcaccaagctggaatacgaccccagatgcgtgtacgaccctagaggccacaccggcaccaccaaccagagaagccctccaggcgagcggaccagaggcagacagcctggcgactacgacgacgacagacggcagcccagaagagaagagggcggcagatggggacctgccggccctagagagagagaacgcgaggaagattggagacagcccagagaggactggcggaggccttctcaccagcagccccggaagatcagacccgagggcagagaaggcgagcaggaatggggcacacctggctctcacgtgcgcgaggaaaccagccggaacaaccccttctacttcccctcccggcggttcagcaccagatacggcaaccagaacggccggatcagagtgctgcagagattcgaccagcggagccggcagttccagaacctgcagaaccaccggatcgtgcagatcgaggccaagcccaacaccctggtgctgcccaaacacgccgacgccgacaacatcctcgtgatccagcagggccaggccaccgtgacagtggccaacggcaacaacagaaagagcttcaacctggacgagggccacgccctgagaatccccagcggcttcatcagctacatcctgaacagacacgacaatcagaacctgagggtggccaagatcagcatgcccgtgaacacccctggccagttcgaggacttcttccccgcatcctcccgggaccagagcagctacctgcagggcttcagccggaataccctggaagccgccttcaacgccgagttcaacgagatcagacgggtgctgctggaagagaacgctggcggagagcaggaagaacggggccagagaagatggtccaccagaagcagcgagaacaacgagggcgtgatcgtgaaggtgtccaaagaacacgtggaagaactgaccaagcacgccaagagcgtgtccaagaagggctccgaggaagagggggacatcaccaaccccatcaatctgagagagggcgagcccgacctgagcaacaacttcggcaagctgttcgaagtgaagcccgacaagaagaacccccagctgcaggacctggacatgatgctgacctgcgtggaaatcaaagagggggccctgatgctgccacacttcaactccaaagccatggtcatcgtggtcgtgaacaagggcaccggcaacctggaactggtggccgtgcggaaagagcagcagcagagaggccgcagagaggaagaagaggacgaggacgaagaagaagagggatccaaccgggaagtgcggcggtacaccgccagactgaaagaaggcgacgtgttcatcatgcctgccgcccaccccgtggccatcaatgcctctagcgagctgcatctgctgggcttcggcattaacgccgagaacaatcaccggatctttctggccggcgacaaagacaacgtgatcgaccagatcgagaagcaggccaaggacctggcctttcccggctctggcgaacaagtggaaaagctgatcaagaaccagaaagaaagccacttcgtgtccgccagaccccagagccagtctcagagccctagctcccccgagaaagagtctcctgagaaagaggaccaggaagaggaaaaccagggcggcaagggccctctgctgagcatcctgaaggccttcaatggcggcggaggcaggcagcagtgggaactgcagggcgacagaagatgccagtcccagctggaacgggccaacctgaggccttgcgagcagcacctgatgcagaaaatccagcgcgacgaggacagctacggccgggatccttacagccccagccaggacccttactcccctagccaggatcccgacagaagggacccctacagccctagcccctacgatagaagaggcgccggaagcagccagcaccaggaaagatgctgcaacgagctgaacgagtttgagaacaaccagcgctgcatgtgcgaggccctgcagcagatcatggaaaatcagagcgaccggctgcagggacggcagcaggaacagcagttcaagagagagctgcggaacctgccccagcagtgtggactgagagccccccagagatgcgacctggaagtggaaagcggcggcagagataggtacggcggagggggcgtgaccttcagacagggcggagaagagaatgagtgccagtttcagcggctgaacgcccagaggcccgacaacagaatcgagagcgagggcggctacatcgagacatggaaccccaacaaccaggaatttcagtgcgctggggtggccctgagcaggaccgtgctgagaagaaatgccctgaggcggcccttctacagcaacgcccccctggaaatctacgtgcagcagggcagcggctacttcggcctgatctttcccggatgcccctccacctatgaggaacccgctcaggaaggcagacggtatcagagccagaagcctagcagacggttccaagtgggccaggacgatcccagccaacagcagcaggactctcaccagaaggtgcaccgcttcgacgagggcgacctgatcgctgtgccaaccggcgtggccttctggatgtacaacgacgaggataccgacgtcgtgaccgtgaccctgagcgacaccagctccatccacaaccagctggaccagttccccaggcggttttacctggccggcaatcaggaacaggaatttctgagataccagcagcagcagggctccagaccccactacagacagatcagccctagagtgcggggcgacgaacaggaaaatgagggcagcaacatcttctccggctttgcccaggaatttctgcagcacgccttccaggtggaccggcagaccgtggaaaacctgagaggcgagaacgagagagaggaacagggcgccatcgtgactgtgaagggcggcctgaggatcctgagccccgacgaagaggatgagtcctctagaagcccccccaaccgccgggaagagttcgatgaggaccgcagcagacctcagcagcgggggaagtacgacgagaacaggcggggctacaagaacggcatcgaggaaacaatctgcagcgccagcgtgaagaagaatctgggccggtccagcaaccccgacatctacaatccacaggccggcagcctgcggagcgtgaacgaactggatctgcccatcctgggatggctgggcctgtctgcccagcacggcaccatctaccggaacgccatgttcgtgcctcactacaccctgaatgcccacaccatcgtggtggctctgaacggccgcgcccacgtccaagtggtggacagcaacggcaatcgggtgtacgatgaagaactgcaggaaggacacgtcctggtggtgccccagaattttgccgtggccgccaaggcccagtccgagaactatgagtatctggccttcaagaccgacagccggccctctatcgccaatcaagccggcgagaacagcatcatcgacaacctgcccgaggaagtggtggccaacagctaccggctgcctagagagcaggcccggcagctgaagaacaacaaccctttcaagttcttcgtgcccccattcgaccaccagagcatgagagaggtggcc

SEQ ID NO: 15 - DNA, 1803 bp, Artificial Sequence
  Description of Artificial Sequence Synthetic polynucleotide
aagtccagcccctaccagaagaaaaccgagaacccctgcgcccagcggtgcctgcagtcttgtcagcaggaacccgacgacctgaagcagaaggcctgcgagagccggtgcaccaagctggaatacgaccccagatgcgtgtacgaccctagaggccacaccggcaccaccaaccagagaagccctccaggcgagcggaccagaggcagacagcctggcgactacgacgacgacagacggcagcccagaagagaagagggcggcagatggggacctgccggccctagagagagagaacgcgaggaagattggagacagcccagagaggactggcggaggccttctcaccagcagccccggaagatcagacccgagggcagagaaggcgagcaggaatggggcacacctggctctcacgtgcgcgaggaaaccagccggaacaaccccttctacttcccctcccggcggttcagcaccagatacggcaaccagaacggccggatcagagtgctgcagagattcgaccagcggagccggcagttccagaacctgcagaaccaccggatcgtgcagatcgaggccaagcccaacaccctggtgctgcccaaacacgccgacgccgacaacatcctcgtgatccagcagggccaggccaccgtgacagtggccaacggcaacaacagaaagagcttcaacctggacgagggccacgccctgagaatccccagcggcttcatcagctacatcctgaacagacacgacaatcagaacctgagggtggccaagatcagcatgcccgtgaacacccctggccagttcgaggacttcttccccgcatcctcccgggaccagagcagctacctgcagggcttcagccggaataccctggaagccgccttcaacgccgagttcaacgagatcagacgggtgctgctggaagagaacgctggcggagagcaggaagaacggggccagagaagatggtccaccagaagcagcgagaacaacgagggcgtgatcgtgaaggtgtccaaagaacacgtggaagaactgaccaagcacgccaagagcgtgtccaagaagggctccgaggaagagggggacatcaccaaccccatcaatctgagagagggcgagcccgacctgagcaacaacttcggcaagctgttcgaagtgaagcccgacaagaagaacccccagctgcaggacctggacatgatgctgacctgcgtggaaatcaaagagggggccctgatgctgccacacttcaactccaaagccatggtcatcgtggtcgtgaacaagggcaccggcaacctggaactggtggccgtgcggaaagagcagcagcagagaggccgcagagaggaagaagaggacgaggacgaagaagaagagggatccaaccgggaagtgcggcggtacaccgccagactgaaagaaggcgacgtgttcatcatgcctgccgcccaccccgtggccatcaatgcctctagcgagctgcatctgctgggcttcggcattaacgccgagaacaatcaccggatctttctggccggcgacaaagacaacgtgatcgaccagatcgagaagcaggccaaggacctggcctttcccggctctggcgaacaagtggaaaagctgatcaagaaccagaaagaaagccacttcgtgtccgccagaccccagagccagtctcagagccctagctcccccgagaaagagtctcctgagaaagaggaccaggaagaggaaaaccagggcggcaagggccctctgctgagcatcctgaaggccttcaat

SEQ ID NO: 16 - DNA, 453 bp, Artificial Sequence
  Description of Artificial Sequence Synthetic polynucleotide
aggcagcagtgggaactgcagggcgacagaagatgccagtcccagctggaacgggccaacctgaggccttgcgagcagcacctgatgcagaaaatccagcgcgacgaggacagctacggccgggatccttacagccccagccaggacccttactcccctagccaggatcccgacagaagggacccctacagccctagcccctacgatagaagaggcgccggaagcagccagcaccaggaaagatgctgcaacgagctgaacgagtttgagaacaaccagcgctgcatgtgcgaggccctgcagcagatcatggaaaatcagagcgaccggctgcagggacggcagcaggaacagcagttcaagagagagctgcggaacctgccccagcagtgtggactgagagccccccagagatgcgacctggaagtggaaagcggcggcagagataggtac

SEQ ID NO: 17 - DNA, 1476 bp, Artificial Sequence
  Description of Artificial Sequence Synthetic polynucleotide
gtgaccttcagacagggcggagaagagaatgagtgccagtttcagcggctgaacgcccagaggcccgacaacagaatcgagagcgagggcggctacatcgagacatggaaccccaacaaccaggaatttcagtgcgctggggtggccctgagcaggaccgtgctgagaagaaatgccctgaggcggcccttctacagcaacgcccccctggaaatctacgtgcagcagggcagcggctacttcggcctgatctttcccggatgcccctccacctatgaggaacccgctcaggaaggcagacggtatcagagccagaagcctagcagacggttccaagtgggccaggacgatcccagccaacagcagcaggactctcaccagaaggtgcaccgcttcgacgagggcgacctgatcgctgtgccaaccggcgtggccttctggatgtacaacgacgaggataccgacgtcgtgaccgtgaccctgagcgacaccagctccatccacaaccagctggaccagttccccaggcggttttacctggccggcaatcaggaacaggaatttctgagataccagcagcagcagggctccagaccccactacagacagatcagccctagagtgcggggcgacgaacaggaaaatgagggcagcaacatcttctccggctttgcccaggaatttctgcagcacgccttccaggtggaccggcagaccgtggaaaacctgagaggcgagaacgagagagaggaacagggcgccatcgtgactgtgaagggcggcctgaggatcctgagccccgacgaagaggatgagtcctctagaagcccccccaaccgccgggaagagttcgatgaggaccgcagcagacctcagcagcgggggaagtacgacgagaacaggcggggctacaagaacggcatcgaggaaacaatctgcagcgccagcgtgaagaagaatctgggccggtccagcaaccccgacatctacaatccacaggccggcagcctgcggagcgtgaacgaactggatctgcccatcctgggatggctgggcctgtctgcccagcacggcaccatctaccggaacgccatgttcgtgcctcactacaccctgaatgcccacaccatcgtggtggctctgaacggccgcgcccacgtccaagtggtggacagcaacggcaatcgggtgtacgatgaagaactgcaggaaggacacgtcctggtggtgccccagaattttgccgtggccgccaaggcccagtccgagaactatgagtatctggccttcaagaccgacagccggccctctatcgccaatcaagccggcgagaacagcatcatcgacaacctgcccgaggaagtggtggccaacagctaccggctgcctagagagcaggcccggcagctgaagaacaacaaccctttcaagttcttcgtgcccccattcgaccaccagagcatgagagaggtggcc

SEQ ID NO: 18 - DNA, 3066 bp, Artificial Sequence
  Description of Artificial Sequence Synthetic polynucleotide
atggcgccccgcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacccttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgttcagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagcaggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcctcgagaagtccagcccctaccagaagaaaaccgagaacccctgcgcccagcggtgcctgcagtcttgtcagcaggaacccgacgacctgaagcagaaggcctgcgagagccggtgcaccaagctggaatacgaccccagatgcgtgtacgaccctagaggccacaccggcaccaccaaccagagaagccctccaggcgagcggaccagaggcagacagcctggcgactacgacgacgacagacggcagcccagaagagaagagggcggcagatggggacctgccggccctagagagagagaacgcgaggaagattggagacagcccagagaggactggcggaggccttctcaccagcagccccggaagatcagacccgagggcagagaaggcgagcaggaatggggcacacctggctctcacgtgcgcgaggaaaccagccggaacaaccccttctacttcccctcccggcggttcagcaccagatacggcaaccagaacggccggatcagagtgctgcagagattcgaccagcggagccggcagttccagaacctgcagaaccaccggatcgtgcagatcgaggccaagcccaacaccctggtgctgcccaaacacgccgacgccgacaacatcctcgtgatccagcagggccaggccaccgtgacagtggccaacggcaacaacagaaagagcttcaacctggacgagggccacgccctgagaatccccagcggcttcatcagctacatcctgaacagacacgacaatcagaacctgagggtggccaagatcagcatgcccgtgaacacccctggccagttcgaggacttcttccccgcatcctcccgggaccagagcagctacctgcagggcttcagccggaataccctggaagccgccttcaacgccgagttcaacgagatcagacgggtgctgctggaagagaacgctggcggagagcaggaagaacggggccagagaagatggtccaccagaagcagcgagaacaacgagggcgtgatcgtgaaggtgtccaaagaacacgtggaagaactgaccaagcacgccaagagcgtgtccaagaagggctccgaggaagagggggacatcaccaaccccatcaatctgagagagggcgagcccgacctgagcaacaacttcggcaagctgttcgaagtgaagcccgacaagaagaacccccagctgcaggacctggacatgatgctgacctgcgtggaaatcaaagagggggccctgatgctgccacacttcaactccaaagccatggtcatcgtggtcgtgaacaagggcaccggcaacctggaactggtggccgtgcggaaagagcagcagcagagaggccgcagagaggaagaagaggacgaggacgaagaagaagagggatccaaccgggaagtgcggcggtacaccgccagactgaaagaaggcgacgtgttcatcatgcctgccgcccaccccgtggccatcaatgcctctagcgagctgcatctgctgggcttcggcattaacgccgagaacaatcaccggatctttctggccggcgacaaagacaacgtgatcgaccagatcgagaagcaggccaaggacctggcctttcccggctctggcgaacaagtggaaaagctgatcaagaaccagaaagaaagccacttcgtgtccgccagaccccagagccagtctcagagccctagctcccccgagaaagagtctcctgagaaagaggaccaggaagaggaaaaccagggcggcaagggccctctgctgagcatcctgaaggccttcaatgaattcacgctgatccccatcgctgtgggtggtgccctggcggggctggtcctcatcgtcctcatcgcctacctcgtcggcaggaagaggagtcacgcaggctaccagactatctag

SEQ ID NO: 19 - DNA, 1716 bp, Artificial Sequence
  Description of Artificial Sequence Synthetic polynucleotide
atggcgccccgcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacccttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgttcagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagcaggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcctcgagaggcagcagtgggaactgcagggcgacagaagatgccagtcccagctggaacgggccaacctgaggccttgcgagcagcacctgatgcagaaaatccagcgcgacgaggacagctacggccgggatccttacagccccagccaggacccttactcccctagccaggatcccgacagaagggacccctacagccctagcccctacgatagaagaggcgccggaagcagccagcaccaggaaagatgctgcaacgagctgaacgagtttgagaacaaccagcgctgcatgtgcgaggccctgcagcagatcatggaaaatcagagcgaccggctgcagggacggcagcaggaacagcagttcaagagagagctgcggaacctgccccagcagtgtggactgagagccccccagagatgcgacctggaagtggaaagcggcggcagagataggtacgaattcacgctgatccccatcgctgtgggtggtgccctggcggggctggtcctcatcgtcctcatcgcctacctcgtcggcaggaagaggagtcacgcaggctaccagactatctag

SEQ ID NO: 20 - DNA, 2739 bp, Artificial Sequence
  Description of Artificial Sequence Synthetic polynucleotide
atggcgccccgcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacccttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgttcagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagcaggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcctcgaggtgaccttcagacagggcggagaagagaatgagtgccagtttcagcggctgaacgcccagaggcccgacaacagaatcgagagcgagggcggctacatcgagacatggaaccccaacaaccaggaatttcagtgcgctggggtggccctgagcaggaccgtgctgagaagaaatgccctgaggcggcccttctacagcaacgcccccctggaaatctacgtgcagcagggcagcggctacttcggcctgatctttcccggatgcccctccacctatgaggaacccgctcaggaaggcagacggtatcagagccagaagcctagcagacggttccaagtgggccaggacgatcccagccaacagcagcaggactctcaccagaaggtgcaccgcttcgacgagggcgacctgatcgctgtgccaaccggcgtggccttctggatgtacaacgacgaggataccgacgtcgtgaccgtgaccctgagcgacaccagctccatccacaaccagctggaccagttccccaggcggttttacctggccggcaatcaggaacaggaatttctgagataccagcagcagcagggctccagaccccactacagacagatcagccctagagtgcggggcgacgaacaggaaaatgagggcagcaacatcttctccggctttgcccaggaatttctgcagcacgccttccaggtggaccggcagaccgtggaaaacctgagaggcgagaacgagagagaggaacagggcgccatcgtgactgtgaagggcggcctgaggatcctgagccccgacgaagaggatgagtcctctagaagcccccccaaccgccgggaagagttcgatgaggaccgcagcagacctcagcagcgggggaagtacgacgagaacaggcggggctacaagaacggcatcgaggaaacaatctgcagcgccagcgtgaagaagaatctgggccggtccagcaaccccgacatctacaatccacaggccggcagcctgcggagcgtgaacgaactggatctgcccatcctgggatggctgggcctgtctgcccagcacggcaccatctaccggaacgccatgttcgtgcctcactacaccctgaatgcccacaccatcgtggtggctctgaacggccgcgcccacgtccaagtggtggacagcaacggcaatcgggtgtacgatgaagaactgcaggaaggacacgtcctggtggtgccccagaattttgccgtggccgccaaggcccagtccgagaactatgagtatctggccttcaagaccgacagccggccctctatcgccaatcaagccggcgagaacagcatcatcgacaacctgcccgaggaagtggtggccaacagctaccggctgcctagagagcaggcccggcagctgaagaacaacaaccctttcaagttcttcgtgcccccattcgaccaccagagcatgagagaggtggccgaattcacgctgatccccatcgctgtgggtggtgccctggcggggctggtcctcatcgtcctcatcgcctacctcgtcggcaggaagaggagtcacgcaggctaccagactatctag

SEQ ID NO: 21 - DNA, 5019 bp, Artificial Sequence
  Description of Artificial Sequence Synthetic polynucleotide
atggcgccccgcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacccttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgttcagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagcaggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcctcgagaagtccagcccctaccagaagaaaaccgagaacccctgcgcccagcggtgcctgcagtcttgtcagcaggaacccgacgacctgaagcagaaggcctgcgagagccggtgcaccaagctggaatacgaccccagatgcgtgtacgaccctagaggccacaccggcaccaccaaccagagaagccctccaggcgagcggaccagaggcagacagcctggcgactacgacgacgacagacggcagcccagaagagaagagggcggcagatggggacctgccggccctagagagagagaacgcgaggaagattggagacagcccagagaggactggcggaggccttctcaccagcagccccggaagatcagacccgagggcagagaaggcgagcaggaatggggcacacctggctctcacgtgcgcgaggaaaccagccggaacaaccccttctacttcccctcccggcggttcagcaccagatacggcaaccagaacggccggatcagagtgctgcagagattcgaccagcggagccggcagttccagaacctgcagaaccaccggatcgtgcagatcgaggccaagcccaacaccctggtgctgcccaaacacgccgacgccgacaacatcctcgtgatccagcagggccaggccaccgtgacagtggccaacggcaacaacagaaagagcttcaacctggacgagggccacgccctgagaatccccagcggcttcatcagctacatcctgaacagacacgacaatcagaacctgagggtggccaagatcagcatgcccgtgaacacccctggccagttcgaggacttcttccccgcatcctcccgggaccagagcagctacctgcagggcttcagccggaataccctggaagccgccttcaacgccgagttcaacgagatcagacgggtgctgctggaagagaacgctggcggagagcaggaagaacggggccagagaagatggtccaccagaagcagcgagaacaacgagggcgtgatcgtgaaggtgtccaaagaacacgtggaagaactgaccaagcacgccaagagcgtgtccaagaagggctccgaggaagagggggacatcaccaaccccatcaatctgagagagggcgagcccgacctgagcaacaacttcggcaagctgttcgaagtgaagcccgacaagaagaacccccagctgcaggacctggacatgatgctgacctgcgtggaaatcaaagagggggccctgatgctgccacacttcaactccaaagccatggtcatcgtggtcgtgaacaagggcaccggcaacctggaactggtggccgtgcggaaagagcagcagcagagaggccgcagagaggaagaagaggacgaggacgaagaagaagagggatccaaccgggaagtgcggcggtacaccgccagactgaaagaaggcgacgtgttcatcatgcctgccgcccaccccgtggccatcaatgcctctagcgagctgcatctgctgggcttcggcattaacgccgagaacaatcaccggatctttctggccggcgacaaagacaacgtgatcgaccagatcgagaagcaggccaaggacctggcctttcccggctctggcgaacaagtggaaaagctgatcaagaaccagaaagaaagccacttcgtgtccgccagaccccagagccagtctcagagccctagctcccccgagaaagagtctcctgagaaagaggaccaggaagaggaaaaccagggcggcaagggccctctgctgagcatcctgaaggccttcaatggcggcggaggcaggcagcagtgggaactgcagggcgacagaagatgccagtcccagctggaacgggccaacctgaggccttgcgagcagcacctgatgcagaaaatccagcgcgacgaggacagctacggccgggatccttacagccccagccaggacccttactcccctagccaggatcccgacagaagggacccctacagccctagcccctacgatagaagaggcgccggaagcagccagcaccaggaaagatgctgcaacgagctgaacgagtttgagaacaaccagcgctgcatgtgcgaggccctgcagcagatcatggaaaatcagagcgaccggctgcagggacggcagcaggaacagcagttcaagagagagctgcggaacctgccccagcagtgtggactgagagccccccagagatgcgacctggaagtggaaagcggcggcagagatcggtacggcggagggggcgtgaccttcagacagggcggagaagagaatgagtgccagtttcagcggctgaacgcccagaggcccgacaacagaatcgagagcgagggcggctacatcgagacatggaaccccaacaaccaggaatttcagtgcgctggggtggccctgagcaggaccgtgctgagaagaaatgccctgaggcggcccttctacagcaacgcccccctggaaatctacgtgcagcagggcagcggctacttcggcctgatctttcccggatgcccctccacctatgaggaacccgctcaggaaggcagacggtatcagagccagaagcctagcagacggttccaagtgggccaggacgatcccagccaacagcagcaggactctcaccagaaggtgcaccgcttcgacgagggcgacctgatcgctgtgccaaccggcgtggccttctggatgtacaacgacgaggataccgacgtcgtgaccgtgaccctgagcgacaccagctccatccacaaccagctggaccagttccccaggcggttttacctggccggcaatcaggaacaggaatttctgagataccagcagcagcagggctccagaccccactacagacagatcagccctagagtgcggggcgacgaacaggaaaatgagggcagcaacatcttctccggctttgcccaggaatttctgcagcacgccttccaggtggaccggcagaccgtggaaaacctgagaggcgagaacgagagagaggaacagggcgccatcgtgactgtgaagggcggcctgaggatcctgagccccgacgaagaggatgagtcctctagaagcccccccaaccgccgggaagagttcgatgaggaccgcagcagacctcagcagcgggggaagtacgacgagaacaggcggggctacaagaacggcatcgaggaaacaatctgcagcgccagcgtgaagaagaatctgggccggtccagcaaccccgacatctacaatccacaggccggcagcctgcggagcgtgaacgaactggatctgcccatcctgggatggctgggcctgtctgcccagcacggcaccatctaccggaacgccatgttcgtgcctcactacaccctgaatgcccacaccatcgtggtggctctgaacggccgcgcccacgtccaagtggtggacagcaacggcaatcgggtgtacgatgaagaactgcaggaaggacacgtcctggtggtgccccagaattttgccgtggccgccaaggcccagtccgagaactatgagtatctggccttcaagaccgacagccggccctctatcgccaatcaagccggcgagaacagcatcatcgacaacctgcccgaggaagtggtggccaacagctaccggctgcctagagagcaggcccggcagctgaagaacaacaaccctttcaagttcttcgtgcccccattcgaccaccagagcatgagagaggtggccgaattcacgctgatccccatcgctgtgggtggtgccctggcggggctggtcctcatcgtcctcatcgcctacctcgtcggcaggaagaggagtcacgcaggctaccagactatctag

SEQ ID NO: 22 - protein, 416 aa, Artificial Sequence
  Description of Artificial Sequence Synthetic polypeptide
MPRQLSAAAALFASLAVILHDGSQMRAKAFPETRDYSQPTAAATVQDIKKPVQQPAKQAPHQTLAARFMDGHITFQTAATVKIPTTTPATTKNTATTSPITYTLVTTQATPNNSHTAPPVTEVTVGPSLAPYSLPPTITPPAHTTGTSSSTVSHTTGNTTQPSNQTTLPATLSIALHKSTTGQKPVQPTHAPGTTAAAHNTTRTAAPASTVPGPTLAPQPSSVKTGIYQVLNGSRLCIKAEMGIQLIVQDKESVFSPRRYFNIDPNATQASGNCGTRKSNLLLNFQGGFVNLTFTKDEESYYISEVGAYLTVSDPETIYQGIKHAVVMFQTAVGHSFKCVSEQSLQLSAHLQVKTTDVQLQAFDFEDDHFGNVDECSSDYTIVLPVIGAIVVGLCLMGMGVYKIRLRCQSSGYQRI

SEQ ID NO: 23 - protein, 408 aa, Artificial Sequence [sequence omitted]

SEQ ID NO: 24 - protein, 478 aa, Artificial Sequence
  Description of Artificial Sequence Synthetic polypeptide
MGRCCFYTAGTLSLLLLVTSVTLLVARVFQKAVDQSIEKKIVLRNGTEAFDSWEKPPLPVYTQFYFFNVTNPEEILRGETPRVEEVGPYTYRELRNKANIQFGDNGTTISAVSNKAYVFERDQSVGDPKIDLIRTLNIPVLTVIEWSQVHFLREIIEAMLKAYQQKLFVTHTVDELLWGYKDEILSLIHVFRPDISPYFGLFYEKNGTNDGDYVFLTGEDSYLNFTKIVEWNGKTSLDWWITDKCNMINGTDGDSFHPLITKDEVLYVFPSDFCRSVYITFSDYESVQGLPAFRYKVPAEILANTSDNAGFCIPEGNCLGSGVLNVSICKNGAPIIMSFPHFYQADERFVSAIEGMHPNQEDHETFVDINPLTGIILKAAKRFQINIYVKKLDDFVETGDIRTMVFPVMYLNESVHIDKETASRLKSMINTTLIITNIPYIIMALGVFFGLVFTWLACKGQGSMDEGTADERAPLIRT

SEQ ID NO: 25 - protein, 197 aa, Artificial Sequence
  Description of Artificial Sequence Synthetic polypeptide
MSRLSRSLLWAATCLGVLCVLSADKNTTQHPNVTTLAPISNVTSAPVTSLPLVTTPAPETCEGRNSCVSCFNVSVVNTTCFWIECKDESYCSHNSTVSDCQVGNTTDFCSVSTATPVPTANSTAKPTVQPSPSTTSKTVTTSGTTNNTVTPTSQPVRKSTFDAASFIGGIVLVLGVQAVIFFLYKFCKSKERNYHTL

SEQ ID NO: 26 - DNA, 918 bp, Artificial Sequence
  Description of Artificial Sequence Synthetic polynucleotide
gtgaccttcagacagggcggagaagagaatgagtgccagtttcagcggctgaacgcccagaggcccgacaacagaatcgagagcgagggcggctacatcgagacatggaaccccaacaaccaggaatttcagtgcgctggggtggccctgagcaggaccgtgctgagaagaaatgccctgaggcggcccttctacagcaacgcccccctggaaatctacgtgcagcagggcagcggctacttcggcctgatctttcccggatgcccctccacctatgaggaacccgctcaggaaggcagacggtatcagagccagaagcctagcagacggttccaagtgggccaggacgatcccagccaacagcagcaggactctcaccagaaggtgcaccgcttcgacgagggcgacctgatcgctgtgccaaccggcgtggccttctggatgtacaacgacgaggataccgacgtcgtgaccgtgaccctgagcgacaccagctccatccacaaccagctggaccagttccccaggcggttttacctggccggcaatcaggaacaggaatttctgagataccagcagcagcagggctccagaccccactacagacagatcagccctagagtgcggggcgacgaacaggaaaatgagggcagcaacatcttctccggctttgcccaggaatttctgcagcacgccttccaggtggaccggcagaccgtggaaaacctgagaggcgagaacgagagagaggaacagggcgccatcgtgactgtgaagggcggcctgaggatcctgagccccgacgaagaggatgagtcctctagaagcccccccaaccgccgggaagagttcgatgaggaccgcagcagacctcagcagcgggggaagtacgacgagaacaggcggggctacaagaac

SEQ ID NO: 27 - DNA, 2181 bp, Artificial Sequence
  Description of Artificial Sequence Synthetic polynucleotide
atggcgccccgcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacccttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgtccagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagccggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcctcgaggtgaccttcagacagggcggagaagagaatgagtgccagtttcagcggctgaacgcccagaggcccgacaacagaatcgagagcgagggcggctacatcgagacatggaaccccaacaaccaggaatttcagtgcgctggggtggccctgagcaggaccgtgctgagaagaaatgccctgaggcggcccttctacagcaacgcccccctggaaatctacgtgcagcagggcagcggctacttcggcctgatctttcccggatgcccctccacctatgaggaacccgctcaggaaggcagacggtatcagagccagaagcctagcagacggttccaagtgggccaggacgatcccagccaacagcagcaggactctcaccagaaggtgcaccgcttcgacgagggcgacctgatcgctgtgccaaccggcgtggccttctggatgtacaacgacgaggataccgacgtcgtgaccgtgaccctgagcgacaccagctccatccacaaccagctggaccagttccccaggcggttttacctggccggcaatcaggaacaggaatttctgagataccagcagcagcagggctccagaccccactacagacagatcagccctagagtgcggggcgacgaacaggaaaatgagggcagcaacatcttctccggctttgcccaggaatttctgcagcacgccttccaggtggaccggcagaccgtggaaaacctgagaggcgagaacgagagagaggaacagggcgccatcgtgactgtgaagggcggcctgaggatcctgagccccgacgaagaggatgagtcctctagaagcccccccaaccgccgggaagagttcgatgaggaccgcagcagacctcagcagcgggggaagtacgacgagaacaggcggggctacaagaacgaattcacgctgatccccatcgctgtgggtggtgccctggcggggctggtcctcatcgtcctcatcgcctacctcgtcggcaggaagaggagtcacgcaggctaccagactatctag

SEQ ID NO: 28 - protein, 912 aa, Artificial Sequence
  Description of Artificial Sequence Synthetic polypeptide
MAPRSARRPLLLLLLLLLLGLMHCASAAMFMVKNGNGTACIMANFSAAFSVNYDTKSGPKNMTLDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENSLEVTFRQGGEENECQFQRLNAQRPDNRIESEGGYIETWNPNNQEFQCAGVALSRTVLRRNALRRPFYSNAPLEIYVQQGSGYFGLIFPGCPSTYEEPAQEGRRYQSQKPSRRFQVGQDDPSQQQQDSHQKVHRFDEGDLIAVPTGVAFWMYNDEDTDVVTVTLSDTSSIHNQLDQFPRRFYLAGNQEQEFLRYQQQQGSRPHYRQISPRVRGDEQENEGSNIFSGFAQEFLQHAFQVDRQTVENLRGENEREEQGAIVTVKGGLRILSPDEEDESSRSPPNRREEFDEDRSRPQQRGKYDENRRGYKNGIEETICSASVKKNLGRSSNPDIYNPQAGSLRSVNELDLPILGWLGLSAQHGTIYRNAMFVPHYTLNAHTIVVALNGRAHVQVVDSNGNRVYDEELQEGHVLVVPQNFAVAAKAQSENYEYLAFKTDSRPSIANQAGENSIIDNLPEEVVANSYRLPREQARQLKNNNPFKFFVPPFDHQSMREVAEFTLIPIAVGGALAGLVLIVLIAYLVGRKRSHAGYQTI

SEQ ID NO: 29 - DNA, 1251 bp, Artificial Sequence
  Description of Artificial Sequence Synthetic polynucleotide
atggcgccccgcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacccttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgtccagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagccggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcacgctgatccccatcgctgtgggtggtgccctggcggggctggtcctcatcgtcctcatcgcctacctcgtcggcaggaagaggagtcacgcaggctaccagactatctag

SEQ ID NO: 30 - protein, 416 aa, Artificial Sequence
  Description of Artificial Sequence Synthetic polypeptide
MAPRSARRPLLLLLLLLLLGLMHCASAAMFMVKNGNGTACIMANFSAAFSVNYDTKSGPKNMTLDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENSTLIPIAVGGALAGLVLIVLIAYLVGRKRSHAGYQTI

SEQ ID NO: 31 - protein, 4 aa, Artificial Sequence
  Description of Artificial Sequence Synthetic peptide
YQTI

SEQ ID NO: 32 - protein, 4 aa, Artificial Sequence
  Description of Artificial Sequence Synthetic peptide
YQRI

SEQ ID NO: 33 - protein, 4 aa, Artificial Sequence
  Description of Artificial Sequence Synthetic peptide
YEQF

SEQ ID NO: 34 - protein, 4 aa, Artificial Sequence
  Description of Artificial Sequence Synthetic peptide
YHTL

SEQ ID NO: 35 - protein, 4 aa, Artificial Sequence
  Description of Artificial Sequence Synthetic peptide
GGGG

SEQ ID NO: 36 - protein, 5 aa, Artificial Sequence
  Description of Artificial Sequence Synthetic peptide
GGGGS

SEQ ID NO: 37 - protein, 4 aa, Artificial Sequence
  Description of Artificial Sequence Synthetic peptide
LIRT

(19) United States
(12) Patent Application Publication
(10) Pub. No.: US 2017/0304432 A1
(43) Pub. Date: Oct. 26, 2017

(54) NUCLEIC ACIDS FOR TREATMENT OF PEANUT ALLERGIES

(71) Applicant: Immunomic Therapeutics, Inc., Rockville, MD (US)

(72) Inventors: William HEARL, Rockville, MD (US); Teri HEILAND, New Market, MD (US)

(21) Appl. No.: 15/321,366

(22) PCT Filed: Jun. 23, 2015
(86) PCT No.: PCT/US15/37240

§ 371 Date: Dec. 22, 2016

Related U.S. Application Data
(60) Provisional application No. 62/015,981, filed on Jun. 23, 2014.

(51) Int. Cl. A61K 39/35; C07K 14/47; C07K 14/415; C07K 14/705; C07K 5/107; A61K 39/00

(52) U.S. Cl. CPC A61K 39/35; C07K 14/415; C07K 14/47; C07K 14/70596; A61K 2039/57; A61K 2039/577; A61K 2039/53; C07K 5/1016

(57) ABSTRACT

Provided herein are DNA vaccines for the treatment of peanut allergies. The vaccines comprise the coding sequence for one or more peanut allergenic epitopes fused in-frame with the luminal domain of the lysosomal associated membrane protein (LAMP) and the targeting sequence of LAMP. The vaccines can be multivalent molecules and/or can be provided as part of a multivalent vaccine comprising two or more DNA constructs.

### CROSS REFERENCE TO RELATED APPLICATIONS

This application claims the benefit of U.S. Appl. No. 62/015,981, filed Jun. 23, 2014, which is incorporated by reference herein in its entirety.

### TECHNICAL FIELD

The disclosed subject matter relates to the fields of molecular biology and medicine. More specifically, the disclosed subject matter relates to nucleic acids for use as DNA vaccines, and methods of using them to treat subjects suffering from or susceptible to peanut allergic reactions.

### BACKGROUND

Allergic reactions occur when the immune system reacts to harmless foreign substances, called allergens. Food allergies are an important public health issue due to the high risk of anaphylaxis, a potentially deadly systemic shock (Sampson et al. (1992) N. Engl. J. Med. 327:380-384; Bock et al. (2001) J. Allergy Clin. Immunol. 107:191-193). Young children are at greater risk of developing food allergies than the general public (Lack et al. (2003) N. Engl. J. Med 348:977-985; Zimmerman et al. (1989) J. Allergy Clin. Immunol. 83:764-770; Green et al. (2007) Pediatrics 120:1304-1310). During the first three years of life, 6-8% of children experience an allergic reaction caused by food (Bock (1987) Allergy 45:587-596; Burks and Sampson (1993) Curr. Prob. Pediatr. 23:230-252; Jansen et al. (1994) J. Allergy Clin. Inmmunol. 93; 2:446-456; Sampson (1999) J. Allergy Chu. Immunol. 103; 5:717-728). Milk, eggs, and peanuts are the three most common food allergens (Sampson (1988) J. Allergy Clin. Immunol. 81:635-645), and by the age of five, 80-85% of children with milk or egg allergies will have outgrown their allergy (Host et al. (1997) J. Allergy Clin. Immunol. 99:S490). In contrast, only 20% of infants develop tolerance to peanut (Skolnick et al. (2001) J. Allergy Clin. Immunol. 107; 2:367-374).

Anaphylaxis caused by exposure to a food allergen, especially peanut, results in a severe immune reaction characterized by overproduction of histamine and is responsible for half of U.S. anaphylaxis emergency room visits annually. Such extreme reactions to peanut result in over 30,000 incidents of anaphylaxis and between 100-200 deaths in the U.S. each year. Peanuts in trace amounts are commonly found in thousands of individually branded, but not labeled, packaged food items. More than one and a half million Americans suffer symptoms from peanut allergy and symptoms often persist throughout life. Many experience dangerous reactions on exposure to trace amounts.

There is no treatment for relieving peanut allergy symptoms; individuals suffering from peanut allergy and institutions like elementary schools must take stringent measures to avoid exposure or ingestion and the risk of a potentially fatal anaphylaxis episode. A diagnosis of peanut allergy requires maintaining constant dietary vigilance to avoid the risk of anaphylaxis (Yunginger et al. (1988) JAMA 260:1450-1452). In the case of children, this vigilance must also be practiced by parents, schools, and care givers. Over the last ten years, the prevalence of peanut allergies has doubled to affect 2% of adult Americans (Sampson (1999) J. Allergy Clin. Immunol. 103; 5:717-728; Sicherer et al. (2003) J. Allergy Clin. Immunol. 112:1203-1207). While the symptoms for many other allergies like hay fever and short ragweed pollen are not life threatening, for a peanut allergic individual, the ingestion of as little as 1/1000th of a peanut can induce anaphylactic shock and death (Taylor et al. (2002) J. Allergy Clin. Immunol. 109 (1):24-30; Wensing et al. (2002) J. Allergy Clin. Immunol. 110(6):915-920). Accidental ingestion of peanuts has been linked to two-thirds of all food-induced anaphylactic shock (Bock et al. (2001) J. Allergy Clin. Immunol. 107:191-193). In the event that accidental ingestion triggers anaphylaxis, injections of epinephrine are used to open up airway passages (Stark and Sullivan (1986) J. Allergy Clin. Immunol. 78:76-83; Sampson (2003) Pediatrics 111(6):1601-1608).

Food allergies occur when an individual fails to develop oral tolerance and instead becomes sensitized to subsequent allergen exposure (Till et al. (2004) J. Allergy Clin. Immunol. 113(6):1025-1034). In allergic patients, allergens preferentially activate type 2 helper CD4+ T lymphocytes (Th2), which produce the pro-allergic cytokines interleukin IL-4, IL-5, and IL-13 that help orchestrate inflammation underlying most allergic symptoms (Woodfolk (2007) J. Allergy Clin. Immunol. 118(2):260-294), IL-4 instructs antibody-producing B cells to secrete allergen-specific Immunoglobulin (Ig) E (Del Prete et al. (1988) J. Immunol. 140:4193-4198; Swain et al. (1990) J. Immunol. 145:3796-3806). Unlike neutralizing IgG, IgE binds to its high affinity receptor Fc-εRI expressed by mast cells and eosinophils (Blank et al. (1989) Nature 337:187-190; Benhamou et al. (1990) J. Immunol. 144:3071-3077), thus sensitizing these cells. Upon subsequent exposure, IgE binds the offending allergen, cross-links, and transduces a signal instructing mast cells to degranulate and release the volatile chemicals that trigger the allergic reaction.

Immunotherapy, the administration of increasing doses of an allergen to bring about tolerance, is a standard treatment for allergic diseases, but has not been approved for treating peanut allergies due to frequent anaphylactic reactions (Nelson et al. (1997) J. Allergy Clin. Immunol 99; 6:744-751; Oppenheimer et. al. (1992) J. Allergy Clin. Immunol 90:256-262). In addition, the utility of immunotherapy is limited by the length of treatment, which requires up to 36 months of weekly or bi-weekly injections and results in varying degrees of success and compliance (Bousquet et al. (1998) J. Allergy Clin. Immunol 102:558-562; Rank and Li (2007) Mayo Clin. Proc. 82(9):1119-1123; Ciprandi et al. (2007) Allergy Asthma Proc. 28:40-43).

### SUMMARY

In one aspect, provided herein is an isolated or purified nucleic acid comprising, in sequential order: a sequence encoding a signal sequence; a sequence encoding an intra-organelle stabilizing/trafficking domain; a sequence encoding a peanut allergen domain, wherein the peanut allergen domain comprises at least one peanut allergen that does not include a naturally-occurring signal sequence for the peanut allergen; a sequence encoding a transmembrane domain; and a sequence encoding an endosomal/lysosomal targeting domain. In some embodiments, the at least one peanut allergen is Ara H1, Ara H2, Ara H3, AraH3del, a portion of Ara H1, Ara H2, or Ara H3 having at least one peanut allergenic epitope, or any combination thereof.

Another aspect provides a pharmaceutical composition comprising one or more isolated or purified nucleic acids comprising in sequential order: a sequence encoding a signal sequence; a sequence encoding an intra-organelle stabilizing/trafficking domain; a sequence encoding a peanut allergen domain, wherein the peanut allergen domain comprises at least one peanut allergen that does not include a naturally-occurring signal sequence for the peanut allergen; a sequence encoding a transmembrane domain; and a sequence encoding an endosomal/lysosomal targeting domain. In some embodiments, the at least one peanut allergen is Ara H1, Ara H2, Ara H3, Ara H3del, a portion of Ara H1, Ara H2, or Ara H3 having at least one peanut allergenic epitope, or any combination thereof.

In still another aspect are provided methods of reducing, eliminating, or preventing an allergic reaction in a subject, the methods comprising administering to the subject a presently disclosed DNA vaccine in an amount sufficient to reduce or eliminate production of an allergen-specific IgE response.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE DRAWINGS

FIG. 1 is a schematic representation of an allergen-LAMP1 protein.

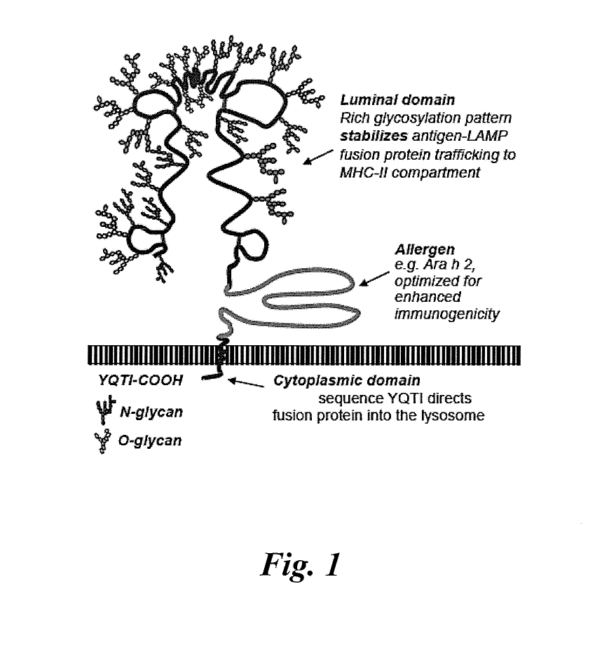

FIG. 2 shows a vector map of a nucleic acid that includes three peanut allergens (AraH1, AraH2, and AraH3, all lacking their native or naturally occurring signal sequences) in the allergen domain.

FIG. 3 shows a schematic of the protein encoded by the nucleic acid of FIG. 2.

FIG. 4 depicts a Western blot showing co-expression of peanut allergens Ara H1, H2, and H3 from a construct according to the present disclosure.

FIG. 5 shows IgG1 antibody levels in mice after immunization with a combination of Ara H1-LAMP, Ara H2-LAMP, and Ara-H3del-LAMP plasmids or a single multivalent-Ara H1/H2/H3-LAMP plasmid (H1-3 multivalent plasmid) by intradermal (ID) or intramuscular (IM) injection; each set of three bars represents the following: left bar, day 49; middle bar, day 70, right bar, day 84 post-immunization.

FIGS. 6A-6B show IgG2a antibody levels in mice after immunization with a combination of Ara. H1-LAMP, Ara H2-LAMP, and Ara-H3del-LAMP plasmids or a single multivalent Ara H1/H2/H3-LAMP plasmid: A) intradermal (ID) injection; each set of three bars represents the following: left bar, day 21; middle bar, day 35; right bar, day 49 post-immunization; and B) intradermal (ID) or intramuscular (IM) injection; blue bar, day 49; red bar, day 70; green bar, day 84 post-immunization.

FIG. 7 shows a representative embodiment of a protocol for the prophylactic studies shown herein.

FIG. 8 shows IgG1 and IgG2a antibody levels in mice after immunization with ARA-LAMP-vax (defined as a combination of Ara H1, Ara H2, and Ara H3del plasmids). Immunization Protocol: five week old female C3H/HeJ mice (N=10 mice/group) were immunized on day 0, 7, and 14 with Ara-LAMP DNA vaccine (wk-3, -2, -1). Control group received LAMP-only vector. CPE is defined as crude peanut extract.

FIG. 9 shows IgG1 and IgG2a antibody levels in mice at day 58 after immunization with ARA-LAMP-vax and sensitization. Sensitization Protocol: mice were sensitized with 10 mg peanut paste (PN)+20 ug cholera toxin (CT), intragastrically (i.g.) three times initially at week (W) 0 and then weekly through W5 followed by two boostings with 50 mg PN+20 ug CT, i.g. at W6 and W8.

FIG. 10 shows IgG1 and IgG2a antibody levels in mice at day 92 after immunization with ARA-LAMP-vax and sensitization. Antibody titers prior to PN challenge, after 5 rounds of PN-CT sensitization—Day 92. Sensitization Protocol continued.

FIG. 11 shows IgG1 and IgG2a antibody levels in mice after immunization with ARA-LAMP-vax, sensitization, and anaphylaxis challenge. Anaphylaxis Challenge: mice were then challenged with 200 mg peanut paste (PN). i.g. at W12.

FIG. 12 shows IgE antibody levels in mice after immunization, sensitization, and anaphylaxis challenge supporting a prophylactic mechanism of ARA-LAMP-vax; each set of two bars represents the following: left bar, Control Vector, right bar, Ara H-LAMP Vaccine; the far left bar that is individually set apart from the other sets of bars represents PreBleed.

FIG. 13 shows a summary of the data shown in FIGS. 9 through 12.

FIG. 14 shows another summary of the data shown in FIGS. 9 through 12; the lower chart shows two lines representing the data points, the top line represents the Control Vector, the bottom line represents ARA-LAMP vax.

FIG. 15 illustrates a representative protocol for the prophylactic studies shown herein.

FIG. 16 shows the IgG-1 (panel A), IgG2a (panel B), and :IgE (panel C) responses to ARA-LAMP-vax or to the single multivalent Ara H1/H2/H3-LAMP plasmid when delivered by intradermal injection (ID) via the Bioject B2000 needle-free device. The 28, 57, 92, 108, 140, and 171 day time points are depicted on the x-axis, each of which shows three bars representing the following: control vector (left bar), combination of 3 plasmids (middle bar) and single multi-allergen plasmid (right bar).

FIG. 17 shows a representative embodiment of a protocol for the therapeutic studies shown herein.

FIG. 18 shows serum peanut specific IgE antibody levels in mice prior to vaccine treatment with ARA-LAMP-vax.

FIG. 19 shows serum peanut specific IgE antibody levels in mice prior to and post vaccine treatment with ARA-LAMP-vax.

FIG. 20 shows anaphylaxis challenge results (symptom score, panel A; body temperature, panel B) in mice at week 15 using ARA-LAMP-vax and a therapeutic protocol.

FIG. 21 shows plasma histamine levels in mice post oral challenge at week 15 using ARA-LAMP-vax and a therapeutic protocol.

FIG. 22 shows IL-4 cytokine levels in mice at week 15 using ARA-LAMP-vax and a therapeutic protocol.

FIG. 23 shows IFN-γ levels in mice at week 15 using ARA-LAMP-vax and a therapeutic protocol.

### DETAILED DESCRIPTION OF EXEMPLARY EMBODIMENTS

Reference will now be made in detail to various exemplary embodiments of the present disclosure. It is to be understood that the following discussion of exemplary embodiments is not intended as a limitation on the invention, as broadly disclosed herein. Rather, the following discussion is provided to give the reader a more detailed understanding of certain aspects and features of the invention. The practice of the present invention employs, unless otherwise indicated, conventional molecular biology, microbiology, and recombinant DNA techniques within the skill of those in the art. Such techniques are explained fully in the literature, are known to the ordinarily skilled artisan in these fields, and thus need not be detailed herein. Likewise, practice of the invention for medical treatment follows standard protocols known in the art, and those protocols need not be detailed herein.

Before embodiments of the present invention are described in detail, it is to be understood that the terminology used herein is for the purpose of describing particular embodiments only, and is not intended to be limiting. Further, where a range of values is provided, it is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention. It is thus to be understood that, where a range of values is presented, each value within that range, and each range falling within that range, is inherently recited as well, and that the avoidance of a specific recitation of each and every value and each and every possible range of values is not an omission of those values and ranges, but instead is a convenience for the reader and for brevity of this disclosure.

Unless defined otherwise, all technical and scientific terms used herein have the same meaning as commonly understood by one of ordinary skill in the art to which the term belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. The present disclosure is controlling to the extent it conflicts with any incorporated publication.

As used herein and in the appended claims, the singular forms “a”, “an”, and “the” include plural referents unless the context clearly dictates otherwise. Thus, for example, reference to “an allergen” includes a plurality of such allergens and reference to “the sample” includes reference to one or more samples and equivalents thereof known to those skilled in the art, and so forth. Furthermore, the use of terms that can be described using equivalent terms include the use of those equivalent terms. Thus, for example, the use of the term “subject” is to be understood to include the terms “animal”, “human”, and other terms used in the art to indicate one who is subject to a medical treatment.

As used herein, the term “comprising” is intended to mean that the constructs, compositions, and methods include the recited elements and/or steps, but do not exclude other elements and/or steps. “Consisting essentially of”, when used to define constructs, compositions, and methods, means excluding other elements and steps of any essential significance to the recited constructs, compositions, and methods. Thus, a composition consisting essentially of the elements as defined herein would not exclude trace contaminants from the isolation and purification method and pharmaceutically acceptable carriers, such as phosphate buffered saline, preservatives, and the like. “Consisting of” means excluding more than trace elements of other ingredients and substantial method steps for administering the compositions of this invention. Embodiments defined by each of these transition terms are within the scope of this invention.

A “chimeric DNA” is an identifiable segment of DNA within a larger DNA molecule that is not found in association with the larger molecule in nature. Thus, when the chimeric DNA encodes a protein segment, the segment coding sequence will be flanked by DNA that does not flank the coding sequence in any naturally occurring genome. In the case where the flanking DNA encodes a polypeptide sequence, the encoded protein is referred to as a “chimeric protein” (i.e., one having non-naturally occurring amino acid sequences fused together). Allelic variations or naturally occurring mutational events do not give rise to a chimeric DNA or chimeric protein as defined herein.

As used herein, the terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA. tRNA, rRNA, ribozymes, antisense molecules, cDNA, recombinant polynucleotides, branched polynucleotides, aptamers, plasmids, vectors, isolated DNA of any sequence, isolated RNA of any sequence, nucleic acid probes, and primers. A nucleic acid molecule may also comprise modified nucleic acid molecules (e.g., comprising modified bases, sugars, and/or internucleotide linkers).

As used herein, the term “peptide” refers to a compound of two or more subunit amino acids, amino acid analogs, or peptidmimetics. The subunits may be linked by peptide bonds or by other bonds (e.g., as esters, ethers, and the like). The term “peptide” is used herein generically to refer to peptides (i.e., polyamino acids of from 2 to about 20 residues), polypeptides (i.e., peptides of from about 20 residues to about 100 residues), and proteins (i.e., peptides having about 100 or more residues).

As used herein, the term “amino acid” refers to either natural and/or unnatural or synthetic amino acids, including glycine and both D or L optical isomers, and amino acid analogs and peptidomimetics. A peptide of three or more amino acids is commonly called an oligopeptide if the peptide chain is short. While the term “protein” encompasses the term “polypeptide”, a “polypeptide” may be a less than a full-length protein.

The term “allergen” refers to any naturally occurring protein or mixtures of proteins that have been reported to induce allergic, i.e., IgE-mediated, reactions upon their repeated exposure to an individual. An allergen is any compound, substance, or material that is capable of evoking an allergic reaction. Allergens are usually understood as a subcategory of antigens, which are compounds, substances, or materials capable of evoking an immune response. For carrying out the invention, the allergen may be selected, among other things, from natural or native allergens, modified natural allergens, synthetic allergens, recombinant allergens, allergoids, and mixtures or combinations thereof. Of particular interest are peanut allergens, especially those that are capable of causing an IgE-mediated immediate type hypersensitivity. In terms of their chemical or biochemical nature, allergens can represent native or recombinant proteins or peptides, fragments or truncated versions of native or recombinant proteins or peptides, fusion proteins, synthetic compounds (chemical allergens), synthetic compounds that mimic an allergen, or chemically or physically altered allergens, such as allergens modified by heat denaturation.

An “epitope” is a structure, usually made up of a short peptide sequence or oligosaccharide, which is specifically recognized or specifically bound by a component of the immune system. T-cell epitopes have generally been shown to be linear oligopeptides. Two epitopes correspond to each other if they can be specifically bound by the same antibody. Two epitopes correspond to each other if both are capable of binding to the same B cell receptor or to the same T cell receptor, and binding of one antibody to its epitope substantially prevents binding by the other epitope (e.g., less than about 30%, preferably, less than about 20%, and more preferably, less than about 10%, 5%, 1%, or about 0.1% of the other epitope binds).

As used herein, two nucleic acid coding sequences “correspond” to each other if the sequences or their complementary sequences encode the same amino acid sequences.

As used herein, a polynucleotide or polynucleotide region (or a polypeptide or polypeptide region) which has a certain percentage (for example, at least about 50%, at least about 60%), at least about 70%, at least about 80%, at least about 85%, at least about 90%, at least about 95%, at least about 99%) of “sequence identity” to another sequence means that, when maximally aligned, manually or using software programs routine in the art, that percentage of bases (or amino acids) are the same in comparing the two sequences.

Two nucleotide sequences are “substantially homologous” or “substantially similar” when at least about 50%, at least about 60%, at least about 70%, at least about 75%, and preferably at least about 80%, and most preferably at least about 90 or 95% of the nucleotides match over the defined length of the DNA sequences. Similarly, two polypeptide sequences are “substantially homologous” or “substantially similar” when at least about 40%, at least about 50%), at least about 60%, at least about 66%, at least about 70%, at least about 75%, and preferably at least about 80%, and most preferably at least about 90 or 95% or 98% of the amino acid residues of the polypeptide match over a defined length of the polypeptide sequence. Sequences that are substantially homologous can be identified by comparing the sequences using standard software available in sequence data banks. Substantially homologous nucleic acid sequences also can be identified in a Southern hybridization experiment under, for example, stringent conditions as defined for that particular system. Defining appropriate hybridization conditions is within the skill of the art. For example, stringent conditions can be: hybridization at 5×SSC and 50%>formamide at 42° C., and washing at 0.1×SSC and 0.1% sodium dodecyl sulfate at 60° C.

“Conservatively modified variants” of domain sequences also can be provided. With respect to particular nucleic acid sequences, the term conservatively modified variants refers to those nucleic acids that encode identical or essentially identical amino acid sequences, or where the nucleic acid does not encode an amino acid sequence, to essentially identical sequences. Specifically, degenerate codon substitutions can be achieved by generating sequences in which the third position of one or more selected (or all) codons is substituted with mixed-base and/or deoxyinosine residues (Balzer et al (1991) Nucleic Acid Res. 19: 508; Ohtsuka et al (1985) J. Biol. Chem. 260: 2605-2608; Rossolini et al (1994) Mol. Cell. Probes 8: 91-98).

The term “biologically active fragment”, “biologically active form”, “biologically active equivalent”, and “functional derivative” of a wild-type protein, means a substance that possesses a biological activity that is at least substantially equal (e.g., not significantly different from) the biological activity of the wild type protein as measured using an assay suitable for detecting the activity. For example, a biologically active fragment comprising a trafficking domain is one which can co-localize to the same compartment as a full length polypeptide comprising the trafficking domain.

A cell has been “transformed”, “transduced”, or “transfected” by exogenous or heterologous nucleic acids when such nucleic acids have been introduced inside the cell.

Transforming DNA may or may not be integrated (covalently linked) with chromosomal DNA making up the genome of the cell. In prokaryotes, yeast, and mammalian cells for example, the transforming DNA may be maintained on an episomal element, such as a plasmid. In a eukaryotic cell, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations (e.g., at least about 10).

A “replicon” is any genetic element (e.g., plasmid, chromosome, virus) that functions as an autonomous unit of DNA replication in vivo.

As used herein, a “viral vector” refers to a virus or viral particle that comprises a polynucleotide to be delivered into a host cell, either in vivo, ex vivo, or in vitro. Examples of viral vectors include, but are not limited to, adenovirus vectors, adeno-associated virus vectors, retroviral vectors, and the like. In aspects where gene transfer is mediated by an adenoviral vector, a vector construct refers to the polynucleotide comprising the adenovirus genome or part thereof, and a selected, non-adeno viral gene, in association with adenoviral capsid proteins.

As used herein, a “nucleic acid delivery vector” is a nucleic acid molecule that can transport a polynucleotide of interest into a cell. Preferably, such a vector comprises a coding sequence operably linked to an expression control sequence. However, a polynucleotide sequence of interest does not necessarily comprise a coding sequence. For example, a polynucleotide sequence of interest can be an aptamer which binds to a target molecule. In another example, the sequence of interest can be a complementary sequence of a regulatory sequence that binds to a regulatory sequence to inhibit regulation of the regulatory sequence. In still another example, the sequence of interest is itself a regulatory sequence (e.g., for titrating out regulatory factors in a cell).

As used herein, a “nucleic acid delivery vehicle” is defined as any molecule or group of molecules or macromolecules that can carry inserted polynucleotides into a host cell (e.g., such as genes or gene fragments, antisense molecules, ribozymes, aptamers, and the like) and that occurs in association with a nucleic acid delivery vector as described above.

As used herein, “nucleic acid delivery” or “nucleic acid transfer” refers to the introduction of an exogenous polynucleotide (e.g., such as a transgene) into a host cell, irrespective of the method used for the introduction. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

As used herein, “expression” refers to the process by which polynucleotides are transcribed into mRNA and/or translated into peptides, polypeptides, or proteins. If the polynucleotide is derived from genomic DNA, expression may include splicing of the mRNA transcribed from the genomic DNA.

As used herein, “under transcriptional control” or “operably linked” refers to expression (e.g., transcription or translation) of a polynucleotide sequence which is controlled by an appropriate juxtaposition of an expression control element and a coding sequence. In one aspect, a DNA sequence is “operatively linked” to an expression control sequence when the expression control sequence controls and regulates the transcription of that DNA sequence.

As used herein, “coding sequence” is a sequence which is transcribed and translated into a polypeptide when placed under the control of appropriate expression control sequences. The boundaries of a coding sequence are determined by a start codon at the 5′ (amino) terminus and a translation stop codon at the 3′ (carboxyl) terminus. A coding sequence can include, but is not limited to, a prokaryotic sequence, cDNA from eukaryotic mRNA, a genomic DNA sequence from eukaryotic (e.g., mammalian) DNA, and even synthetic DNA sequences. For example, such synthetic DNA sequences may include those that are codon optimized for the organism in which the sequences are intended to be expressed. A polyadenylation signal and transcription termination sequence will usually be located 3′ to the coding sequence.

As used herein, a “genetic modification” refers to any addition to or deletion or disruption of a cell's normal nucleotide sequence. Art-recognized methods include viral mediated gene transfer, liposome mediated transfer, transformation, transfection and transduction, e.g., viral-mediated gene transfer such as the use of vectors based on DNA viruses such as adenovirus, adeno-associated virus and herpes virus, as well as retroviral based vectors.

As used herein, “the lysosomal/endosomal compartment” refers to membrane-bound acidic vacuoles containing LAMP molecules in the membrane, hydrolytic enzymes that function in antigen processing, and MHC class II molecules for antigen recognition and presentation. This compartment functions as a site for degradation of foreign materials internalized from the cell surface by any of a variety of mechanisms including endocytosis, phagocytosis, and pinocytosis, and of intracellular material delivered to this compartment by specialized autolytic phenomena (see, for example, de Duve (1983) Eur. J. Biochem. 137: 391). The term “endosome” as used herein encompasses a lysosome.

As used herein, a “lysosome-related organelle” refers to any organelle that comprises lysozymes and includes, but is not limited to, MIIC, CUV, melanosomes, secretory granules, lytic granules, platelet-dense granules, basophil granules, Birbeck granules, phagolysosomes, secretory lysosomes, and the like. Preferably, such an organelle lacks mannose 6-phosphate receptors and comprises a LAMP, but might or might not comprise an MHC class II molecule. For reviews, see, e.g., Blott and Griffiths (2002) Nature Reviews, Molecular Cell Biology; DellAngelica et al. (2000) The FASEB Journal 14: 1265-1278.

As used herein, a “lysosomal associated membrane protein” or “LAMP” refers to any protein comprising a domain found in the membrane of an endosomal/lysosomal compartment or lysosome-related organelle and which further comprises a luminal domain. Exemplary LAMPs include but are not limited to LAMP-1, LAMP-2, CD63/LAMP-3 (DC-LAMP), or homologs, orthologs, variants (e.g., allelic variants) and modified forms (e.g., comprising one or more mutations, either naturally occurring or engineered) thereof. In some embodiments, a LAMP is a mammalian lysosomal associated membrane protein, e.g., such as a human or mouse lysosomal associated membrane protein. Exemplary LAMPs include a peptide comprising an amino acid sequence which is at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, or 100% identical to SEQ ID NOs: 22, 23, 24, 25, or 30. Exemplary nucleotide sequences encoding LAMP peptides that may be used in accordance with the disclosed nucleic acid molecules include but are not limited to any sequence that is at least about 80% identical, at least about 81% identical, at least about 82% identical, at least about 83% identical, at least about 84% identical, at east about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical to, or 100% identical to SEQ ID NO: 10, 11, 12, 13, or 29.

As used herein, the term “stabilizing domain” is intended to mean a domain that aids in keeping a protein active and/or in its natural conformation.

As used herein, the term “trafficking domain” is intended to mean a domain that aids in targeting a protein to a specific part of a cell.

As used herein, “targeting domain” denotes the polypeptide sequence that directs a chimeric protein of the invention to a preferred site, such as a cellular organelle or compartment where antigen processing and binding to MHC II occurs. As such, a “targeting domain” refers to a series of amino acids that are required for delivery to a cellular compartment/organelle. Preferably, a targeting domain is a sequence that binds to an adaptor or AP protein (e.g., such as an AP1, AP2, or AP3 protein). Exemplary targeting domain sequences are described in DellAngelica, 2000, for example.

As used herein, an “endosomal/lysosomal targeting domain” refers to a series of amino acids that are required for delivery to an endosomal/lysosomal compartment or lysosome-related organelle. For example, LAMP trafficking to the outer membrane of lysosomes is mediated by binding of adaptor proteins to an endosomal/lysosomal targeting domain, which is a tyrosine recognition sequence (YXXØ) in the carboxy-terminal cytoplasmic tail (where Y is a tyrosine residue, X can be any amino acid and Ø is a large hydrophobic residue). Exemplary tyrosine recognition sequences include the amino acid sequences YQTI, YQRI, YEQF, and YHTL.

As used herein, in vivo nucleic acid delivery, nucleic acid transfer, nucleic acid therapy, and the like, refer to the introduction of a vector comprising an exogenous polynucleotide directly into the body of an organism, such as a human or non-human mammal, whereby the exogenous polynucleotide is introduced into a cell of such organism in vivo.

As used herein, the term “in situ” refers to a type of in vivo nucleic acid delivery in which the nucleic acid is brought into proximity with a target cell (e.g., the nucleic acid is not administered systemically). For example, in situ delivery methods include, but are not limited to, injecting a nucleic acid directly at a site (e.g., into a tissue, such as a tumor or heart muscle), contacting the nucleic acid with cell(s) or tissue through an open surgical field, or delivering the nucleic acid to a site using a medical access device such as a catheter.

As used herein, the terms “isolated” and “purified” are used at times interchangeably to mean separated from constituents, cellular and otherwise, with which the polynucleotide, peptide, polypeptide, protein, antibody, or fragments thereof, are normally associated in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragments thereof, does not require “isolation” to distinguish it from its naturally occurring counterpart. Furthermore, the terms “isolated” and “purified” do not imply total isolation and total purity. These terms are used to denote both partial and total purity from some or all other substances naturally found in association with the polynucleotide, etc. Thus, these terms can mean isolation or purification from one naturally associated substance (e.g., isolation or purification of DNA from RNA), isolation or purification from other substances of the same general class of molecule (e.g., a particular protein showing 20% purity as compared to all proteins in a sample), or any combination. Isolation and purification can mean any level from about 1% to about 100%, including 100%. As such, an “isolated” or “purified” population of cells is substantially free of cells and materials with which it is associated in nature. Of course, those of skill in the art will recognize that all specific values, including fractions of values, are encompassed within these ranges without the need for each particular value to be listed herein. Each value is not specifically disclosed for the sake of brevity; however, the reader is to understand that each and every specific value is inherently disclosed and encompassed by the invention.

As used herein, a “target cell” or “recipient cell” refers to an individual cell or cell which is desired to be, or has been, a recipient of an exogenous nucleic acid molecule, polynucleotide, and/or protein. The term is also intended to include progeny of a single cell, and the progeny may not necessarily be completely identical (in morphology or in genomic or total DNA complement) to the original parent cell due to natural, accidental, or deliberate mutation. A target cell may be in contact with other cells (e.g., as in a tissue) or may be found circulating within the body of an organism.

The term “antigen presenting cell” or “APC” as used herein refers to any cell that presents on its surface an antigen in association with a major histocompatibility complex molecule, or portion thereof, or, alternatively, one or more non-classical MHC molecules, or a portion thereof. Examples of suitable APCs are discussed in detail below and include, but are not limited to, whole cells such as macrophages, dendritic cells, B cells, hybrid APCs, and foster antigen presenting cells.

As used herein an “engineered antigen-presenting cell” refers to an antigen-presenting cell that has a non-natural molecular moiety on its surface. For example, such a cell may not naturally have a co-stimulator on its surface or may have additional artificial co-stimulator in addition to natural co-stimulator on its surface, or may express a non-natural class II molecule on its surface.

As used herein, the term “immune effector cell” refers to a cell that is capable of binding an antigen and that mediates an immune response. These cells include, but are not limited to, T cells, B cells, monocytes, macrophages, NK cells, and cytotoxic T lymphocytes (CTLs), for example CTL lines, CTL clones, and CTLs from tumor, inflammatory, or other infiltrates.

As used herein, the terms “subject” and “patient” are used interchangeably to indicate an animal for which the present invention is directed. The term animal is to be understood to include humans and non-human animals; where a distinction between the two is desired, the terms human and/or non-human animal are used. In some embodiments, the subject or patient is a vertebrate, preferably a mammal, more preferably a human. Mammals include, but are not limited to, murines, simians, humans, farm animals (e.g., bovines, ovines, porcines), sport animals (e.g. equines), and pets (e.g., canines and felines).

Clinical allergy symptoms are known to those of skill in the art, and an exhaustive listing herein is not required. Non-limiting examples include rhinitis, conjunctivitis, asthma, urticaria, eczema, which includes reactions in the skin, eyes, nose, upper and lower airways with common symptoms such as redness and itching of eyes and nose, itching and runny nose, coaching, wheezing, shortness of breath, itching, and swelling of tissue.

Examples of “immunological in vivo tests” are Skin Prick Test (SPT), Conjunctival Provocation Test (CPT), Bronchial Challenge with Allergen (BCA), and various clinical tests in which one or more allergy symptoms is monitored. See, for example, Haugaard et al., J Allergy Clin Immunol, Vol. 91, No. 3, pp 709-722, March 1993.

As used herein, the term “pharmaceutically acceptable carrier” encompasses any of the standard pharmaceutical carriers known in the art, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin Remington's Pharm. Sci., 15th Ed. (Mack Publ. Co., Easton (1975)).

As used herein, a “therapeutically effective amount” is used herein to mean an amount sufficient to prevent, correct, and/or normalize an abnormal physiological response. In one aspect, a “therapeutically effective amount” is an amount sufficient to reduce by at least about 30 percent, more preferably by at least 50 percent, most preferably by at least 90 percent, a clinically significant feature of pathology, such as for example, clinical allergy symptoms, antibody production, cytokine production, fever or white cell count, or level of histamine.

An “antibody” is any immunoglobulin, including antibodies and fragments thereof, that binds a specific epitope. The term encompasses polyclonal, monoclonal, and chimeric antibodies (e.g., bispecific antibodies). An “antibody combining site” is that structural portion of an antibody molecule comprised of heavy and light chain variable and hypervariable regions that specifically binds antigen. Exemplary antibody molecules are intact immunoglobulin molecules, substantially intact immunolgobulin molecules, and those portions of an immunoglobulin molecule that contains the paratope, including Fab, Fab′, F(ab′)₂, and F(v) portions, which portions are preferred for use in the therapeutic methods described herein.

The term “oromucosal administration” refers to a route of administration where the dosage form is placed under the tongue or anywhere else in the oral cavity to allow the active ingredient to come in contact with the mucosa of the oral cavity or the pharynx of the patient in order to obtain a local or systemic effect of the active ingredient. An example of an oromucosal administration route is sublingual administration. The term “sublingual administration” refers to a route of administration where a dosage form is placed underneath the tongue in order to obtain a local or systemic effect of the active ingredient. As used herein, the term “intradermal delivery” means delivery of the vaccine to the dermis in the skin. However, the vaccine will not necessarily be located exclusively in the dermis. The dermis is the layer in the skin located between about 1.0 and about 2.0 mm from the surface in human skin, but there is a certain amount of variation between individuals and in different parts of the body. In general, it can be expected to reach the dermis by going 1.5 mm below the surface of the skin. The dermis is located between the stratum corneum and the epidermis at the surface and the subcutaneous layer below. Depending on the mode of delivery, the vaccine may ultimately be located solely or primarily within the dermis, or it may ultimately be distributed within the epidermis and the dermis.

As used herein, the term “prevent” or “prophylactically” in the context of allergy immunotherapy, allergy treatment, or other terms that describe an intervention designed for an allergy patient, means the prevention of an IgE response in at least 20% of all patients. The term “prevent” does not require total prevention from developing an IgE mediated disease in all patients, and such a definition is outside the scope of the present invention for treating allergy through a mechanism that reduces allergy symptoms, and is inconsistent with the use of the term in the art. It is well known to those skilled in the art of allergy immunotherapy that allergy treatments are not 100% effective in 100% of patients, and as such an absolute definition of “prevent” does not apply within the context of the present invention. The art-recognized concept of prevention is contemplated by the present invention.

Broadly speaking, the present disclosure provides polynucleic acids, polyaminoacids, and methods of treating subjects in need of the polynucleic acids and polyaminoacids. The polynucleic acids and compositions thereof can be thought of as nucleic acid (e.g., DNA, RNA) vaccines for the intracellular production of peanut allergenic sequences (polyaminoacids) that elicit a protective immune response within the body of the subject to whom the polynucleic acid is administered. The polynucleic acids, when administered, preferentially evoke a cell-mediated immune response via the MHC-II pathway and production of IgG antibodies by activating a peanut allergen-specific T-helper type 1 (Th1) cellular response with the production of interferons by APCs, NK cells, and T cells rather than a Th2-type response, which involves production of IgE antibodies, granulocytes (e.g., eosinophils), and other substances. To an extent, both an MHC-II and an MHC-I response can be generated; however, the invention provides a response that is primarily or substantially an MHC-II response. In some embodiments, the immune system is rebalanced in favor of an IgG/Th1 response instead of an allergic IgE Th2 response. Preferably, the nucleic acids do not encode an antibiotic resistance gene.

Specifically, provided herein are novel peanut allergy DNA vaccines that utilize a lysosomal associated membrane protein (“LAMP”) chimeric construct to direct peanut allergens into the MHC/endosomal pathway. The disclosed vaccines, including the nucleic acid molecules, vectors, and pharmaceutical compositions described herein, provide peanut allergy sufferers with a safe, hypoallergenic, and cost-effective therapy that significantly reduces or eliminates sensitivity to peanuts.

The disclosed nucleic acids, when administered to a subject, sequester the antigen into the lysosomal compartment of antigen presenting cells and effect a Th2 to Th1 response modulation in allergic patients. Another advantage is that the presently disclosed constructs have been designed to prevent accidental allergen exposure. The allergen is encoded as a nucleic acid so no significant amount of allergen is exposed systemically upon administration. It is encoded within a LAMP for high fidelity lysosomal trafficking. In the lysosome, the allergen undergoes proteolysis, exposing allergenic epitopes to and presentation to helper T-cells. Thus, a subject receiving the presently disclosed therapy shows a clinical response without exposure to free allergen.

Accordingly, in some embodiments are provided an isolated or purified nucleic acid molecule comprising, in sequential order: a nucleic acid sequence encoding a signal sequence; a nucleic acid sequence encoding an intra-organelle stabilizing/trafficking domain; a nucleic acid sequence encoding a peanut allergen domain, which can comprise a single peanut allergen or two or more peanut allergens, each comprising one or more peanut allergenic epitopes, and wherein the at least one peanut allergen does not include a native signal sequence for the peanut allergen; a nucleic acid sequence encoding a transmembrane domain; and a nucleic acid sequence encoding an endosomal/lysosomal targeting domain.

The isolated or purified nucleic acid molecules provided herein comprise a signal sequence. In some embodiments, the signal sequence comprises a signal sequence of a LAMP. In some embodiments, the signal sequence is an endoplasmic reticulum translocation sequence. Exemplary LAMP signal sequences include, but are not limited to, the signal sequence of LAMP-1, LAMP2, LAMP-3 (DC-LAMP), LIMP II, or ENDOLYN. Exemplary LAMP signal sequences include a peptide comprising an amino acid sequence which is at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, or 100% identical to amino acids 1-27 of SEQ ID NO: 1, amino acids 1-27 of SEQ ID NO: 22, amino acids 1-28 of SEQ ID NO: 23, amino acids 5-27 of SEQ ID NO: 24 and amino acids 1-24 of SEQ ID NO: 25. Exemplary nucleotide sequences encoding LAMP signal sequences that may be used in accordance with the disclosed nucleic acid molecules include but are not limited to any sequence that is at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical to, or 100% identical to nucleotides 1-86 of SEQ ID NO: 18, nucleotides 1-86 of SEQ ID NO: 19, nucleotides 1-86 of SEQ ID NO: 20, nucleotides 1-86 of SEQ ID NO: 21, nucleotides 1-84 of SEQ ID NO: 10, nucleotides 1-81 of SEQ ID NO: 11, nucleotides 1-72 of SEQ ID NO: 12 and nucleotides 13-81 of SEQ ID NO: 13.

The isolated or purified nucleic acid molecule described herein further comprise a sequence encoding the intra-organelle stabilizing/trafficking domain comprising a sequence encoding a lysosomal associated membrane protein (LAMP). For example, the intra-organelle stabilizing/trafficking domain may comprise a luminal domain of a LAMP. In another embodiment, the intra-organelle stabilizing/trafficking domain comprises a luminal domain of the LAMP1, LAMP2, LAMP-3 (DC-LAMP),LIMP II, or ENDOLYN. Exemplary intra-organelle stabilizing/trafficking domains include but are not limited to an amino acid sequence which is at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, or 100% identical to amino acids 28 to 380 of SEQ ID NO: 1, amino acids 28-381 of SEQ ID NO: 22, amino acids 29-375 of SEQ ID NO: 23, amino acids 28-433 of SEQ ID NO: 24, or amino acids 25-162 of SEQ ID NO: 25. Exemplary intra-organelle stabilizing/trafficking domains may be encoded by a nucleotide sequence that is at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical to, or 100% identical to nucleotides 87-1146 of SEQ ID NO: 18, nucleotides 87-1146 of SEQ ID NO: 19, nucleotides 87-1146 of SEQ ID NO: 20, nucleotides 87-1146 of SEQ ID NO: 21, nucleotides 85-1125 of SEQ ID NO: 10, nucleotides 82-1143 of SEQ ID NO: 11, nucleotides 73-486 of SEQ ID NO: 12, or nucleotides 82-1299 of SEQ ID NO: 13.

The nucleic acid molecules described herein further comprise a sequence encoding a peanut allergen domain. The sequence encoding the peanut allergen domain comprises a nucleic acid sequence encoding one or more peanut allergen proteins, polypeptides, or peptides, which comprises one or more allergenic epitopes. The peanut allergen domain preferably does not include the naturally occurring signal sequences from the peanut allergen(s). Where less than a full-length peanut allergenic sequence is used, preferably, one or more epitopes of the full-length peanut allergen protein are provided in the context of their natural positions within the allergenic protein. The peanut allergen domain can include two or more allergens, each containing one or more allergenic epitopes. In still other embodiments, the sequence encoding a peanut allergen domain comprises a sequence that encodes three peanut allergens. It is known that certain allergenic proteins contain two or more epitopes. In some embodiments, the sequence encoding the peanut allergen domain comprises an entire allergenic coding region (i.e., the coding region lacking a signal sequence), or a substantial portion thereof, of a peanut allergenic protein. Some peanut allergen domains will include two or more epitopes in their naturally-occurring relationship. Alternatively, two or more known peanut allergenic epitopes can be fused into one coding region. Yet again, in exemplary embodiments, two or more peanut allergenic proteins, or allergenic regions thereof, are present in the peanut allergen domain. Where two or more epitopes are engineered to be present in a single epitope domain, the epitopes can be from the same antigenic protein. In some embodiments, the isolated or purified nucleic acid molecule comprises a nucleic acid sequence comprising a nucleic acid sequence that encodes two or more peanut allergenic epitopes. In yet another embodiment, the nucleic acid sequence encoding a peanut allergen domain comprises a nucleic acid sequence that encodes two or more peanut allergens. In yet another embodiment, the nucleic acid sequence encoding a peanut allergen domain comprises a nucleic acid sequence that encodes three peanut allergens. In some embodiments, the at least one peanut allergen is Ara H1, Ara H2, Ara H3, AraH3del, a portion thereof having at least one peanut allergenic epitope, or any combination thereof.

In some embodiments, the nucleic acid sequence encoding the at least one peanut allergen domain comprises a nucleotide sequence that is at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical to, or 100% identical to SEQ ID NO: 14, SEQ ID NO: 15, SEQ ID NO: 16, SEQ ID NO: 17, SEQ ID NO: 26, nucleotides 1147-2943 of SEQ ID NO: 18, nucleotides 1147-1600 of SEQ ID NO 19 , nucleotides 1147-2623 of SEQ ID NO: 20, nucleotides 1147-2949 of SEQ ID NO: 21, nucleotides 2962-3414 of SEQ ID NO: 21, and/or nucleotides 3427-4902 of SEQ ID NO: 21. In some embodiments, the Ara H peanut allergen domain comprises an amino acid sequence which is at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, or 100% identical to SEQ ID NO:2, SEQ 1D NO:3, SEQ ID NO:4, SEQ 1D NO:5, amino acids 383 to 983 of SEQ ID NO: 1, amino acids 988 to 1138 of SEQ ID NO: 1, amino acids 1143 to 1634 of SEQ ID NO: 1, and/or amino acids 383 to 1634 of SEQ ID NO: 1.

In some embodiments, the peanut allergenic epitopes or peanut allergens are separated by a linker. For example, the linker may comprise the amino acid sequence GGGG or GGGGS.

The isolated or purified nucleic acid molecules provided herein further comprise a transmembrane domain. Transmembrane domains are well characterized physical and functional elements of proteins that exist partially on both sides of a biological membrane. Generally, a transmembrane domain is a linear sequence of amino acids that are hydrophobic or lipophilic in nature and which function to anchor a protein at a biological membrane. Such sequences are often 20-25 residues in length. Those of skill in the art are well aware of such sequences and can easily obtain or engineer a suitable transmembrane sequence for use in the present invention. In some embodiments, the transmembrane domain comprises a transmembrane domain of a LAMP, for example but not limited to LAMP-1, LAMP2, LAMP-3 (DC-LAMP), LIMP II, or ENDOLYN. Exemplary nucleotide sequences encoding a transmembrane domain that may be used in accordance with the disclosed nucleic acid molecules include but are not limited to any sequence that is at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical to, or 100% identical to nucleotides 1126-1188 of SEQ ID NO: 10, nucleotides 1144-1212 of SEQ ID NO: 11, nucleotides 487-555 of SEQ ID NO: 12, nucleotides 1300-1395 of SEQ ID NO: 13, or nucleotides 1141-1212 of SEQ ID NO: 29. Exemplary LAMP transmembrane domains include an amino acid sequence which is at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, or 100% identical to amino acids 376-396 of SEQ ID NO: 23, amino acids 382-404 of SEQ ID NO: 22, amino acids 434-466 of SEQ ID NO: 24, amino acids 163-185 of SEQ ID NO: 25, or amino acids 381-404 of SEQ ID NO: 30.

The nucleic acid molecules further comprise a nucleic acid sequence encoding an endosomal/lysosomal targeting domain. In some embodiments, the endosomal/lysosomal targeting domain may be a tyrosine recognition sequence (YXXØ signal) in the carboxy-terminal cytoplasmic tail (where Y is a tyrosine residue, X can be any amino acid and Ø is a large hydrophobic residue). In some embodiments, the tyrosine recognition sequence (YXXØ signal) comprises the amino acid sequence YQTI, YQRI, YEQF, or YHTL. The nucleic acid sequence encoding an endosomal/lysosomal targeting domain may comprise nucleotides 5005-5016 of SEQ ID NO: 21, nucleotides 1213-1224 of SEQ ID NO: 10, nucleotides 1237-1248 of SEQ ID NO: 11, or nucleotides 580-591 of SEQ ID NO: 12. In some embodiments, the nucleic acid sequence encoding an endosomal/Lysosomal targeting domain may comprise nucleotides 1420-1431 of SEQ ID NO: 13.

In some embodiments, the disclosed nucleic acid molecules comprise a nucleotide sequence that is at least about 80% identical, at least about 81% identical, at least about 82% identical at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical at least about 95% identical, at least about 96% identical, at least about 97% identical, at least about 98% identical, at least about 99% identical to, or 100% identical to SEQ ID NO: 18, SEQ ID NO: 19, SEQ ID NO: 20, SEQ ID NO: 21, or SEQ ID NO: 27. The disclosed nucleic acid molecules provided herein may comprise a nucleic acid sequence encoding an amino acid sequence which is at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, or 100% identical to SEQ ID NO: 1, SEQ ID NO:6, SEQ ID NO:7, SEQ ID NO:8, or SEQ ID NO: 28.

The disclosed nucleic acid molecule can comprise deoxyribonucleic acid (DNA).

In some embodiments, the isolated or purified nucleic acid comprising, in sequential order, a sequence encoding a signal sequence; a sequence encoding an intra-organelle stabilizing/trafficking domain; a sequence encoding a peanut allergen domain, which can comprise a single peanut allergen or two or more peanut allergens, each comprising one or more peanut allergenic epitopes, and wherein the at least one peanut allergen does not include a naturally-occurring signal sequence for the peanut allergen; a sequence encoding a transmembrane domain; and a sequence encoding an endosomal/lysosomal targeting domain, is present on a single chimeric or engineered nucleic acid. The sequences encoding the respective domains of the disclosed isolated or purified nucleic acids can be combined in any order using techniques known and widely practiced in the art. In some embodiments, the domains are combined and arranged such that they comprise a single open reading frame encoding a chimeric protein, the open reading frame being operably linked to transcriptional elements sufficient for expression of the chimeric protein. The nucleic acid thus can include an expression vector, such as a plasmid, phagemid, viral vector, or the like. Preferably, the nucleic acid comprises transcriptional elements suitable for expression in mammalian cells, such as human cells.

In some embodiments, the present disclosure provides peanut allergens, e.g. Ara H1, Ara H2, Ara H3, and/or AraH3del within one plasmid. As a representative example, the single multivalent Ara H1/H2/H3 LAMP plasmid disclosed herein comprises the major peanut allergens, Ara H1, Ara H2, and Ara H3 in a single plasmid. Also provided herein is a single multivalent AraH1/H2/H3del LAMP plasmid. The plasmid may also comprise combinations of two peanut allergens, e.g. Ara H1 and Ara H2, Ara H2 and Ara H3, or Ara H1and Ara H3 in a single plasmid.

In some embodiments, the present disclosure provides peanut allergens, e.g. Ara H1, Ara H2, AraH3, and/or Ara H3del, each on its own plasmid. As a representative example, the ARA-LAMP vax composition comprises the major peanut allergens, Ara H1, Ara H2, and Ara H3del encoded by separate plasmids. Also provided herein is a composition comprising Ara H1, Ara H2, and Ara H3 encoded by separate plasmids. In such instances, the composition comprises a mixture of at least two DNA vaccines, where each vaccine comprises the sequence of one peanut allergen. The vaccine constructs can be mixed together at a ratio of 1:1, 1:2, 1:3, 1:4, sequentially up to 1:10 (e.g., 1:5, 1:6, 1:7, 1:8 and 1:9). The preferred ratio is 1:1.

In some embodiments, a presently disclosed nucleic acid is a DNA vaccine that induces an immune response in a host. In some embodiments, the DNA vaccine comprises the previously described isolated or purified nucleic acid comprising, in sequential order: a sequence encoding a signal sequence; a sequence encoding an intra-organelle stabilizing/trafficking domain; a sequence encoding a peanut allergen domain, which can comprise a single peanut allergen or two or more peanut allergens, each comprising one or more peanut allergenic epitopes, and wherein the at least one peanut allergen does not include a naturally-occurring signal sequence for the peanut allergen; a sequence encoding a transmembrane domain; and a sequence encoding an endosomal/lysosomal targeting domain. In some embodiments, the DNA vaccine comprises at least two isolated or purified nucleic acids comprising in sequential order: a sequence encoding a signal sequence; a sequence encoding an intra-organelle stabilizing/trafficking domain; a sequence encoding a peanut allergen domain, which can comprise a single peanut allergen or two or more peanut allergens, each comprising one or more peanut allergenic epitopes, and wherein the at least one peanut allergen does not include a naturally-occurring signal sequence for the peanut allergen; a sequence encoding a transmembrane domain; and a sequence encoding an endosomal/lysosomal targeting domain. In some embodiments, the DNA vaccine comprises at least three isolated or purified nucleic acids comprising, in sequential order: a sequence encoding a signal sequence; a sequence encoding an intra-organelle stabilizing/trafficking domain; a sequence encoding a peanut allergen domain, which can comprise a single peanut allergen or two or more peanut allergens, each comprising one or more peanut allergenic epitopes, and wherein the at least one peanut allergen does not include a naturally-occurring signal sequence for the peanut allergen; a sequence encoding a transmembrane domain; and a sequence encoding an endosomal/lysosornal targeting domain.

Further provided are host cells that express the nucleic acids or vectors provided herein. In some embodiments, the host cells are mammalian host cells, preferably human cells.

Also provided herein are polypeptides comprising a signal sequence; an intra-organelle stabilizing/trafficking domain; a peanut allergen domain, which can comprise a single peanut allergen or two or more peanut allergens, each comprising one or more peanut allergenic epitopes, and wherein the at least one peanut allergen does not include a naturally-occurring signal sequence for the peanut allergen; a transmembrane domain; and an endosomal/lysosomal targeting domain.

In some embodiments of the polypeptides provided herein, the signal sequence comprises a signal sequence of a LAMP. In some embodiments, the signal sequence is an endoplasmic reticulum translocation sequence. Exemplary LAMP signal sequences include, but are not limited to, the signal sequence of LAMP-1, LAMP2, LAMP-3 (DC-LAMP), LIMP II, or ENDOLYN. Exemplary LAMP signal sequences include a peptide comprising an amino acid sequence which is at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, or 100% identical to amino acids 1-27 of SEQ ID NO: 1, amino acids 1-27 of SEQ ID NO: 22, amino acids 1-28 of SEQ ID NO: 23, amino acids 5-27 of SEQ ID NO: 24 and amino acids 1-24 of SEQ ID NO: 25.

The polypeptides further comprise an intra-organelle stabilizing/trafficking domain. For example, the intra-organelle stabilizing/trafficking domain may comprise a luminal domain of a LAMP. In another embodiment, the intra-organelle stabilizing/trafficking domain comprises a luminal domain of LAMP1, LAMP2, LAMP-3 (DC-LAMP), LIMP II, or ENDOLYN. Exemplary intra-organelle stabilizing/trafficking domains include but are not limited to an amino acid sequence which is at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, or 100% identical to amino acids 28 to 380 of SEQ ID NO: 1, amino acids 28-381 of SEQ ID NO: 22, amino acids 29-375 of SEQ ID NO: 23, amino acids 28-433 of SEQ ID NO: 24, or amino acids 25-162 of SEQ ID NO: 25.

The polypeptides described herein further comprise a peanut allergen domain. The peanut allergen domain comprises one or more peanut allergen proteins, polypeptides, or peptides, which comprises one or more allergenic epitopes. The peanut allergen domain preferably does not include the naturally occurring signal sequences from the peanut allergen(s). Where less than a full-length peanut allergenic sequence is used, preferably, one or more epitopes of the full-length peanut allergen protein are provided in the context of their natural positions within the allergenic protein. The peanut allergen domain can include two or more allergens, each containing one or more allergenic epitopes. In still other embodiments, the peanut allergen domain comprises three peanut allergens. It is known that certain allergenic proteins contain two or more epitopes. Some peanut allergen domains will include two or more epitopes in their naturally-occurring relationship. Where two or more epitopes are engineered to be present in a single epitope domain, the epitopes can be from the same antigenic protein. In some embodiments, the at least one peanut allergen is Ara H1, Ara H2, Ara H3, AraH3del, a portion thereof having at least one peanut allergenic epitope, or any combination thereof.

In some embodiments, the Ara H peanut allergen domain comprises an amino acid sequence which is at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, or 100% identical to SEQ ID NO:2, SEQ ID NO:3, SEQ ID NO:4, SEQ ID NO:5, amino acids 383 to 983 of SEQ ID NO: 1, amino acids 988 to 1138 of SEQ ID NO: 1, amino acids 1143 to 1634 of SEQ ID NO: 1, and/or amino acids 383 to 1634 of SEQ ID NO: 1.

In some embodiments, the peanut allergenic epitopes or peanut allergens are separated by a linker. For example, the linker may comprise the amino acid sequence GGGG or GGGGS.

The polypeptides provided herein further comprise a transmembrane domain. In some embodiments, the transmembrane domain comprises a transmembrane domain of a LAMP, for example but not limited to LAMP-1, LAMP2, LAMP-3 (DC-LAMP), LIMP II, or ENDOLYN. Exemplary LAMP transmembrane domains include an amino acid sequence which is at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, or 100% identical to amino acids 1637 to 1660 of SEQ ID NO: 1, amino acids 376-396 of SEQ ID NO: 23, amino acids 382-404 of SEQ ID NO: 22, amino acids 434-466 of SEQ ID NO: 24, amino acids 163-185 of SEQ ID NO: 25, or amino acids 381-404 of SEQ ID NO: 30.

The described polypeptides further comprise an endosomal/lysosomal targeting domain. In some embodiments, the endosomal/lysosomal targeting domain may comprise a tyrosine recognition sequence (YXXØ signal) in the carboxy-terminal cytoplasmic tail (where Y is a tyrosine residue, X can be any amino acid and Ø is a large hydrophobic residue). In some embodiments, the tyrosine recognition sequence (YXXØ signal) comprises the amino acid sequence YQTI, YQRI, YEQF, or YHTL. In some embodiments, the endosomal/lysosomal targeting domain comprises the amino acid sequence LIRT.

In some embodiments, the described polypeptides comprise an amino acid sequence which is at least about 80% identical, at least about 81% identical, at least about 82% identical, at least about 83% identical, at least about 84% identical, at least about 85% identical, at least about 86% identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, or 100% identical to SEQ ID NO: 1, SEQ ID NO:6, SEQ ID NO:7, SEQ ID NO:8, or SEQ ID NO: 28.

In some embodiments are provided pharmaceutical compositions comprising at least one of the presently disclosed nucleic acid molecules. Also provided are pharmaceutical compositions comprising at least one presently disclosed vector. In some embodiments, the pharmaceutical compositions may comprise a vector comprising a nucleic acid encoding SEQ ID NO: 1, SEQ ID NO: 6, SEQ ID NO: 7, SEQ ID NO: 8, or SEQ ID NO: 28. For example, the nucleic acid may be encoded by SEQ ID NO: 20, SEQ ID NO: 21, SEQ ID NO: 27, SEQ ID NO: 19, or SEQ ID NO: 18. In some embodiments are provided pharmaceutical compositions comprising at least two presently disclosed nucleic acid molecules or vectors. In still other embodiments are provided pharmaceutical compositions comprising at least three presently disclosed nucleic acid molecules or vectors. For example, the at least three disclosed nucleic acid molecules may comprise a nucleic acid molecule encoding SEQ ID NO: 6, a nucleic acid molecule encoding SEQ ID NO: 7, and a nucleic acid molecule encoding SEQ ID NO: 8. Exemplary nucleic acid sequences include SEQ ID NO: 27, SEQ ID NO: 19, and SEQ ID NO: 18. In yet another embodiment, the pharmaceutical composition comprises at least two vectors, each comprising a presently disclosed nucleic acid molecule. Further provided herein are pharmaceutical compositions comprising a first, second, and third vector, wherein the first vector comprises a nucleic acid molecule encoding SEQ ID NO: 6, the second vector comprises a nucleic acid molecule encoding SEQ ID NO: 7, and the third vector comprises a nucleic acid molecule encoding SEQ ID NO: 8. Exemplary nucleic acid sequences include SEQ ID NO: 20, SEQ ID NO: 27 , SEQ ID NO: 19, and SEQ ID NO: 18.

In some embodiments, the presently disclosed pharmaceutical composition further comprises a pharmaceutically acceptable carrier. The nucleic acids or vectors of the present disclosure can be provided as a purified or isolated molecule. The nucleic acids or vectors also can be provided as part of a composition. The compositions can consist essentially of the nucleic acid or vector, meaning that the nucleic acid or vector is the only nucleic acid or vector in the composition suitable for expression of a coding sequence. Alternatively, the composition can comprise a nucleic acid or vector as disclosed herein. In exemplary embodiments, the composition is a pharmaceutical composition comprising the nucleic acid or vector as disclosed herein along with one or more pharmaceutically acceptable substances or carriers, e.g., saline. In some embodiments, the composition comprises a substance that promotes uptake of the nucleic acid by a cell. In some embodiments, the composition comprises a targeting molecule that assists in delivering the nucleic acid to a specific cell type, such as an immune cell (e.g., APC or Antigen Presenting Cell). In other embodiments, the nucleic acid is part of a delivery vehicle or delivery vector for delivery of the nucleic acid to a cell or tissue. In preferred embodiments, the presently disclosed formulations comprise naked DNA in, for example, saline. In other preferred embodiments, the DNA vaccine is delivered by intramuscular (IM) or intradermal (ID) injection.

The present disclosure also provides methods for using the presently disclosed nucleic acid molecules, vectors and pharmaceutical compositions. In some embodiments, the present disclosure provides a method of preventing or treating a peanut allergic reaction in a subject in need thereof, comprising administering a therapeutically effective amount of a presently disclosed nucleic acid molecule, vector or pharmaceutical composition to the subject. In some embodiments, the nucleic acid molecule, vector or pharmaceutical composition is administered in an amount sufficient to decrease the production of an IgE response. In some embodiments, the nucleic acid molecule, vector or pharmaceutical composition is administered in an amount sufficient to decrease plasma histidine levels. In some embodiments, the nucleic acid molecule, vector or pharmaceutical composition is administered in an amount sufficient to decrease the production of IL-4. In some embodiments, the nucleic acid molecule, vector or pharmaceutical composition is administered in an amount sufficient to increase IFN-γ levels. In some embodiments, the method reduces, eliminates, or prevents at least one clinical allergy symptom. In some embodiments, the nucleic acid molecule, vector or pharmaceutical composition is administered to the subject by intramuscular (IM) injection. In some embodiments, the nucleic acid molecule, vector or pharmaceutical composition is administered to the subject by intradermal (ID) injection. In some embodiments, the nucleic acid molecule, vector or pharmaceutical composition is administered in an amount sufficient to induce or increase the production of an allergen-specific IgG response . In some embodiments, the nucleic acid molecule, vector or pharmaceutical composition is administered in an amount sufficient to attenuate an IgE response. In some embodiments, the subject is a human.

In some embodiments, the present disclosure provides a method of preventing or treating a peanut allergic reaction in a subject in need thereof, comprising administering a therapeutically effective amount of a presently disclosed nucleic acid molecule, vector or pharmaceutical composition to the subject, wherein the subject was exposed to a peanut allergen prior to the administering. In some embodiments, the nucleic acid molecule, vector or pharmaceutical composition is administered in an amount sufficient to decrease the production of an IgE response. In some embodiments, the nucleic acid molecule, vector or pharmaceutical composition is administered in an amount sufficient to decrease plasma histidine levels. In some embodiments, the nucleic acid molecule, vector or pharmaceutical composition is administered in an amount sufficient to decrease the production of IL-4. In some embodiments, the nucleic acid molecule, vector or pharmaceutical composition is administered in an amount sufficient to increase IFN-γ levels. In some embodiments, the method reduces, eliminates, or prevents at least one clinical allergy symptom. In some embodiments, the nucleic acid molecule, vector or pharmaceutical composition is administered to the subject by intramuscular (IM) injection. In some embodiments, the nucleic acid molecule, vector or pharmaceutical composition is administered to the subject by intradermal (ID) injection. In some embodiments, the nucleic acid molecule, vector or pharmaceutical composition is administered in an amount sufficient to induce or increase the production of an allergen-specific IgG response. In some embodiments, the nucleic acid molecule, vector or pharmaceutical composition is administered in an amount sufficient to attenuate an IgE response. In some embodiments, the subject is a human.

In some embodiments, the present disclosure provides a method of preventing or treating a peanut allergic reaction in a subject in need thereof, comprising administering a therapeutically effective amount of a presently disclosed nucleic acid molecule, vector or pharmaceutical composition to the subject, wherein the subject is a human. In some embodiments, the nucleic acid molecule, vector or pharmaceutical composition is administered in an amount sufficient to decrease the production of an IgE response. In yet further embodiments, the nucleic acid molecule, vector or pharmaceutical composition is administered in an amount sufficient to decrease plasma histidine levels. In some embodiments, the nucleic acid molecule, vector or pharmaceutical composition is administered in an amount sufficient to decrease the production of IL-4. In some embodiments, the nucleic acid molecule, vector or pharmaceutical composition is administered in an amount sufficient to increase IFN-γ levels. In some embodiments, the method reduces, eliminates, or prevents at least one clinical allergy symptom. In some embodiments, the nucleic acid molecule, vector or pharmaceutical composition is administered to the subject by intramuscular (IM) injection. In some embodiments, the nucleic acid molecule, vector or pharmaceutical composition is administered to the subject by intradermal (ID) injection. In some embodiments, the nucleic acid molecule, vector or pharmaceutical composition is administered in an amount sufficient to induce or increase the production of an allergen-specific IgG response. In some embodiments, the nucleic acid molecule, vector or pharmaceutical composition is administered in an amount sufficient to attenuate an IgE response. In some embodiments, the subject is a human.

In some embodiments, the present disclosure provides a method of preventing or treating a peanut allergic reaction in a subject in need thereof, comprising administering a therapeutically effective amount of a presently disclosed nucleic acid molecule, vector or pharmaceutical composition to the subject, wherein the subject was exposed to a peanut allergen prior to the administering, wherein the subject is a human. In some embodiments, the nucleic acid molecule, vector or pharmaceutical composition is administered in an amount sufficient to decrease the production of an IgE response. In some embodiments, the nucleic acid molecule, vector or pharmaceutical composition is administered in an amount sufficient to decrease plasma histidine levels. In some embodiments, the nucleic acid molecule, vector or pharmaceutical composition is administered in an amount sufficient to decrease the production of IL-4. In some embodiments, the nucleic acid molecule, vector or pharmaceutical composition is administered in an amount sufficient to increase IFN-γ levels. In some embodiments, the method reduces, eliminates, or prevents at least one clinical allergy symptom. In some embodiments, the nucleic acid molecule, vector or pharmaceutical composition is administered to the subject by intramuscular (IM) injection. In some embodiments, the nucleic acid molecule, vector or pharmaceutical composition is administered to the subject by intradermal (ID) injection. In some embodiments, the nucleic acid molecule, vector or pharmaceutical composition is administered in an amount sufficient to induce or increase the production of an allergen-specific IgG response. In some embodiments, the nucleic acid molecule, vector or pharmaceutical composition is administered in an amount sufficient to attenuate an IgE response. In some embodiments, the subject is a human.

In other embodiments, the method comprises administering to the subject a presently disclosed nucleic acid, vector, pharmaceutical composition, or DNA vaccine in an amount sufficient to induce or increase the production of an allergen-specific IgG response. In yet other embodiments, the method prevents the peanut allergic reaction. In still other embodiments, the method reduces, eliminates, or prevents at least one clinical allergy symptom. In further embodiments, the DNA vaccine is administered prophylactically to the subject to prevent a peanut allergic reaction. In still further embodiments, the DNA vaccine is administered therapeutically to the subject to treat a peanut allergic reaction.

Methods of treating subjects in need using the presently disclosed vaccines are also provided by this disclosure. In some embodiments, the methods are methods of prophylactically treating or therapeutically treating a subject at risk of developing or a subject suffering from an allergic reaction to one or more peanut allergens. In other embodiments, the methods comprise administering to the subject a DNA vaccine according to the invention in an amount sufficient to cause uptake of and expression of the DNA vaccine by an APC. Without limiting the invention to a particular mechanism of action, expression of the DNA vaccine results in presentation of the encoded allergenic epitope(s) on the APC, and development of an IgG immune response.

In a particular instance of the invention, a nucleic acid sequence encoding SEQ ID NO: 1, SEQ ID NO:2, SEQ ID NO:3, SEQ ID NO:4, SEQ ID NO:5, SEQ NO:6, SEQ H) NO:7, SEQ ID NO:8, or SEQ ID NO: 28, a portion of at least one of these sequences, and/or another peanut allergen encoding sequence is administered to a cell. In another particular instance of the invention, at least two peanut allergens found on separate DNA constructs are administered in combination to a cell. In preferred embodiments, the cell is an antigen presenting cell, such as a dendritic cell. Preferably, the dendritic cell is a human dendritic cell. The present invention can be administered by methods known in the art to be effective delivery methods for nucleic acid vaccines, including intramuscular injection, intradermal injection, subcutaneous injection, electroporation, gene gun vaccination, or liposome-mediated transfer.

The present invention provides a formulation that when administered to a cell results in an increased specific antibody response. The increased antibody response to the peanut allergen is useful for treating an IgE-mediated allergic disease. IgE has certain properties related to its cellular restriction and the resulting intracellular signaling upon binding cognate allergen. IgE is generated against a peanut allergen when B cells receive IL-4 secreted by Th2 cells. This helps instruct B cells to produce IgE class antibodies. Upon secretion by B cells, IgE binds to Fc-eRI, its high affinity receptor expressed by mast cells and eosinophils, resulting in these cells and the animal becoming sensitized to future allergen exposure. Consequently, the symptoms of allergy can be triggered upon the ingestion, inhalation, or mucosal contact with a peanut allergen. Due to the binding properties of antibodies, it has been proposed that one way of reducing peanut allergy symptoms is to chelate free allergen available for binding by IgE through competition with other antibody classes. In particular, an allergy formulation that increases IgG has been proposed to be a pathway for reducing allergic disease. The invention described herein induces enhanced IgG production, thus causing a decrease in the ratio of IgE to IgG in a clinically significant manner.

### EXAMPLES

The invention will now be described with reference to exemplary embodiments of the invention. The following examples are intended to give the reader a better understanding of the construction and activity of the constructs of the invention, and should not be construed as a limitation on the scope of the invention.

### Example 1

### General Materials and Methods

Genetic sequences were prepared that encoded the peanut allergens Ara H1, Ara H2, and Ara H3 as the native sequences (control plasmids) and as chimeras with human LAMP-1 (experimental plasmids), with each sequence inserted between the luminal and transmembrane domains of LAMP. Previous studies have shown that antigenic sequences must be optimized for human usage, thus all unnecessary or deleterious elements (cryptic splice sites, secondary RNA/DNA structures, secondary ORFs) were removed in order to maximize RNA stability and protein expression. AraH1-LAMP comprised SEQ ID NO: 15. AraH2-LAMP comprised SEQ ID NO: 12. The final optimized sequence was chemically synthesized and inserted into the LAMP open reading frame (ORF) of the antibiotic free pDNA-VACC-ultra vector (Nature Pharmaceuticals, Lincoln, Nebr.). The expression of the chimeric protein was determined for each plasmid by transfecting NIH3T3 cells and subsequent Western blot analysis. Cellular trafficking to the lysosome was confirmed by confocal microscopy and by immunoblotting cell lysates.

The AraH3del gene was codon optimized for human usage using the GeneArt/Invitrogen online gene design software. The synthetic gene was manufactured by GeneArt/Invitrogen (Life Technologies, Grand Island, N.Y.). The synthetic gene was inserted into the N LAMP-C LAMP gene to create N LAMP-AraH3del-C LAMP (SEQ ID NO: 27) which was then inserted into the expression vector. The deletion was created based on the proteolytic processing of the native AraH3 protein into an acidic and basic subunit. The acidic subunit was generated and used as a single plasmid.

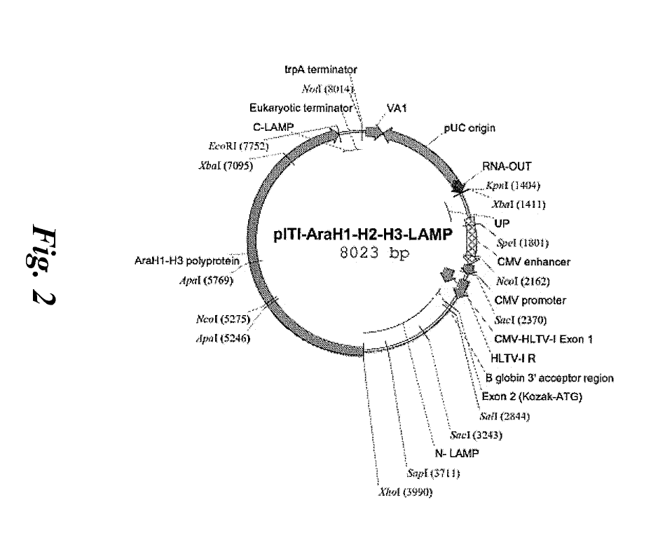

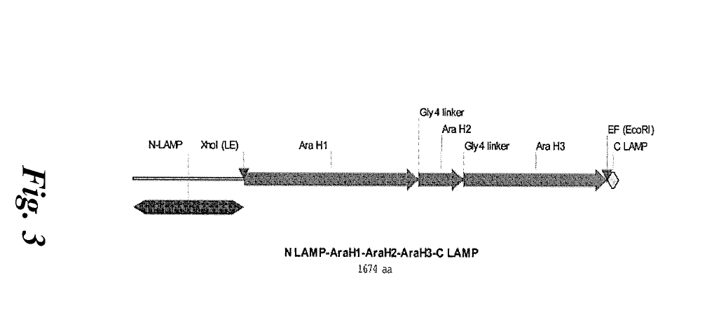

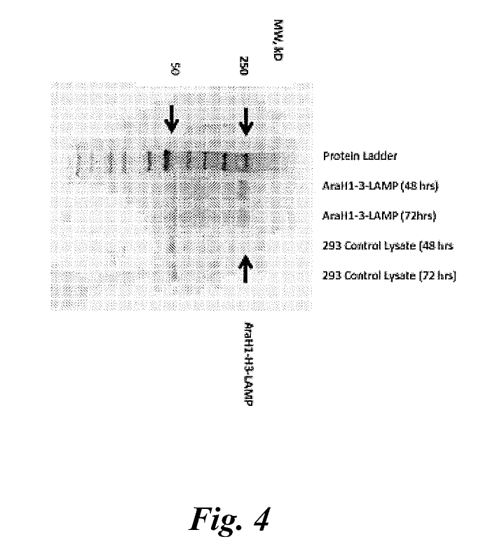

The single multivalent construct (AraH1/H2/H3-LAMP comprising SEQ ID NO: 21) was prepared by synthesizing DNA encoding each of the dominant peanut allergens (Ara H1, Ara H2, Ara H3) inserted into a LAMP-vax immunization vector (FIGS. 2 and 3). In this single multivalent peanut construct, a 5 amino acid linker sequence (GGGGS) was inserted in between Ara H1 and Ara H2 and in between Ara H2 and Ara H3. Western blot analysis showed co-expression of peanut allergens Ara H1, H2, and H3 from the Ara-LAMP-vax single multivalent construct (FIG. 4). The ARA-LAMP vax composition comprised three plasmids, each comprising DNA encoding a single peanut allergen inserted into a LAMP-vax immunization vector (Ara H1, H2, or H3del) within the luminal and transmembrane domain of LAMP (i.e., SEQ ID NOs: 18, 19, and 20) in the pDNAVACC-ultra vector; Nature Technology Corp., Lincoln, Nebr.). Ara-LAMP-vax is also referred to herein as Ara-H-LAMP. It was established that each plasmid expressed the chimeric Ara/LAMP protein in transfected cell culture and that mice generated allergen-specific antibodies as a result of treatment. The expression of each vector was assessed in transfected cells singularly and in combination.

All animal experiments were conducted in compliance with the animal ethics committee of the Office of Laboratory Animal Welfare (OLAW) approved facility. BALB/c mice were immunized with either with the Ara-LAMP-vax single multivalent construct or the Ara-LAMP-vax three-plasmid composition by intramuscular (IM) or intradermal (ID) injection and the immune response was characterized. Control mice were immunized with blank vector (i.e., pDNAVACC-ultra vector without the Ara-LAMP construct; “control vector”) at the same concentration. The day prior to antigen challenge, mice were prepared for passive cutaneous anaphylaxis (PCA) tests as previously described (Saloga et al (1993) J. Clin. Invest. 91(1):133-40; Li et al (1999) J. Immunol. 162:5624-5630).

To determine therapeutic efficacy, naïve mice were sensitized to peanut and then immunized two weeks later with an Ara-LAMP-vax formulation (50 μg of single multivalent Ara-LAMP-vax plasmid/200 μL PBS per animal or 50 μg of each of AraH1-LAMP-vax plasmid, AraH2-LAMP-vax plasmid, and AraH3-LAMP-vax plasmid/200 μL PBS per animal) three times in two week intervals. Following immunization, mice were challenged with peanut by experimentally inducing food allergy through the administration of 10 mg of Peanut Paste (PN) with 20 μg of cholera toxin (CT) using a ball-ended mouse feeding needle once a week for 8 weeks and scored for allergy symptoms.

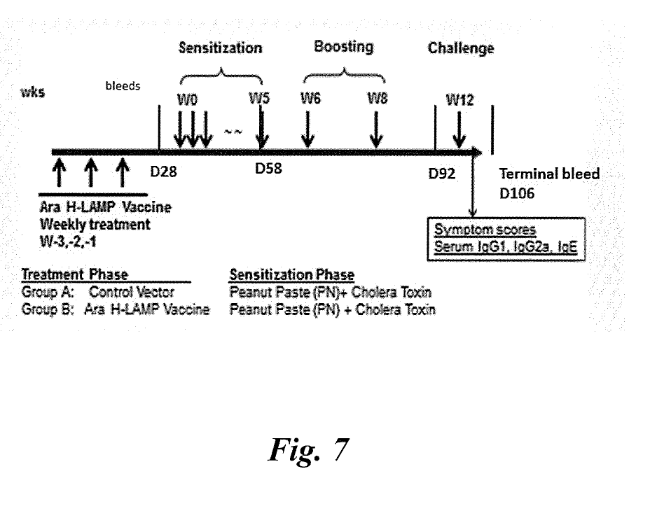

To determine prophylactic efficiency, peanut naïve BALB/c mice were immunized three times with Ara-LAMP-vax (50 μg of single multivalent Ara-LAMP-vax plasmid/200 μL PBS per animal or a formulation of 50 μg of each of AraH1-LAMP-vax plasmid, AraH2-LAMP-vax plasmid, and AraH3-LAMP-vax plasmid/200 μL PBS per animal) at day 0, day 14 and day 28, and then sensitized with peanut extract and cholera toxin (FIG. 7). Serum samples were collected at each vaccination date and then weekly until day 42.

Upon antigen challenge, allergy symptoms were scored by blinded, independent investigators according to a 0-5 scale, where 0 represented no symptoms and 5 was death (Li et al. (2001) J. Allergy Clin. Immunol. 108:639-646). To determine the histamine levels, sera was collected and assayed 30-40 minutes after challenge. Histological studies were performed on ear samples using light microscopy to determine the degree of mast cell degranulation as a result of systemic anaphylaxis.

Mice were bled weekly and the sera were stored at −80° C. Mice were sacrificed, the immune response generated by each vaccine formulation was assayed, and antibody levels for IgG subtypes, IgE and cytokines were measured. The immunological response of T-cells and B-cells was evaluated by means of ELISPOT, ELISA, cell proliferation assays, and cytokine assays. To determine if any vaccine formulation results in allergen leakage, serum samples were assayed for peanut allergens by sandwich ELISA.

For the cytokine assays, supernatants were assayed for the presence of IFN-γ and IL-4 by ELISA. Matched antibody pairs were used for IFN-γ and IL-4 and done according to manufacturer's instructions. The standard curves were generated with mouse recombinant IFN-gamma and IL-4. All antibodies and cytokines were purchased from Invitrogen, Carlsbad, Calif. The detection limits of IFN-γ and IL-4 assays were 20 and 10 μg/ml, respectively.

### Example 2

### ARA-LAMP Prophylactic Studies

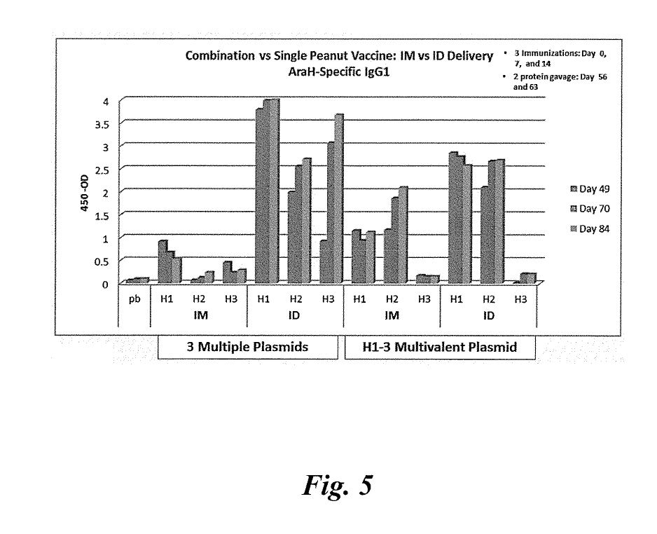

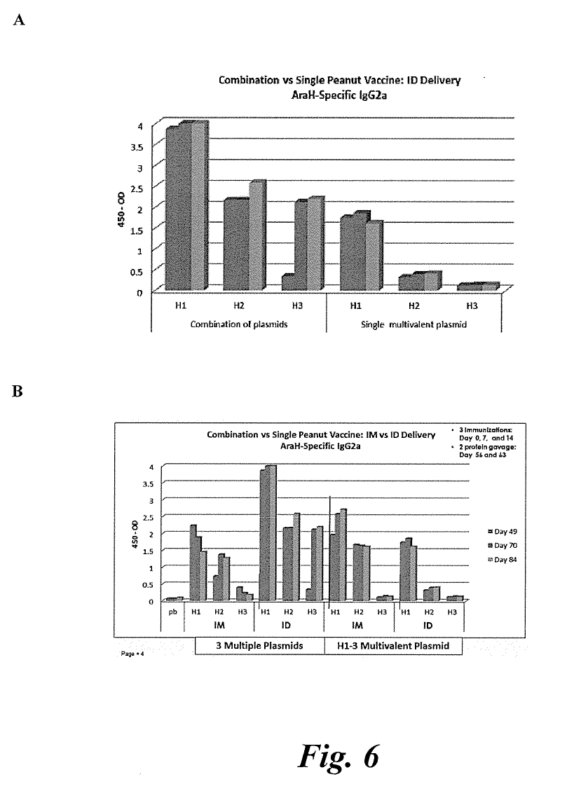

DNA constructs comprising the peanut allergens Ara H1, Ara H2, and/or Ara H3 were tested in a mouse model for prophylactic effectiveness. A multivalent LAMP plasmid (AraH1/H2/H3-LAMP generated according to Example 1) encoding the peanut allergens Ara H1, Ara2, and Ara H3 was compared to a three plasmid mix (AraH1-LAMP, AraH2-LAMP, and ARAH3del-LAMP, prepared in accordance with Example 1), each plasmid encoding a single peanut allergen. BALB/c mice were immunized with either 50 μg of the single multivalent peanut plasmid or 50 μg of each individual plasmid weekly for three weeks either by intradermal (ID) or intramuscular (IM) injection. Five weeks following the last immunization, IgG1 and IgG2a antibody titers were assayed by ELISA. The multivalent plasmid was found to be immunogenic, but the magnitude of antibody response was lower than single allergen delivery in multiple plasmids (FIGS. 5 and 6). Without wishing to be bound to any one particular theory, it is believed that antigen competition, such as epitope access to MHC-II presentation, may limit the immune response to all antigens. The strongest response after immunization was with the multiple plasmids delivered by intradermal (ID) injection.

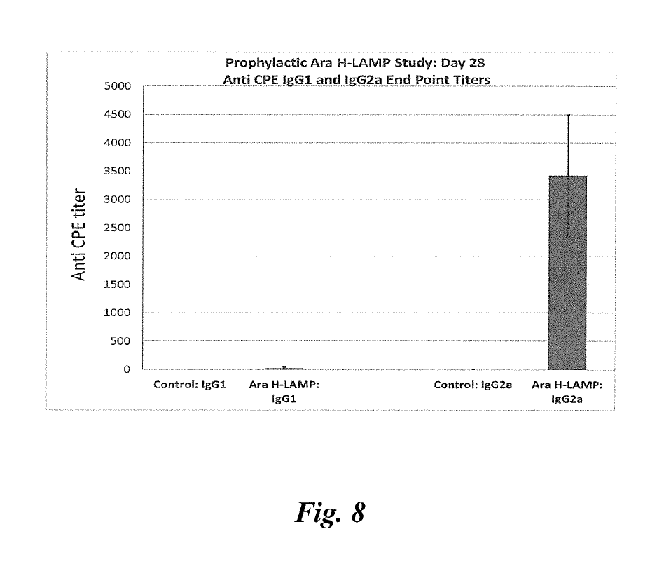

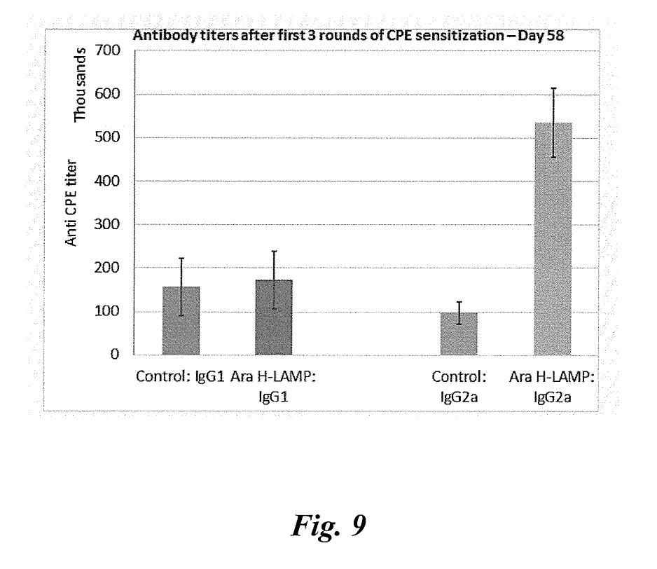

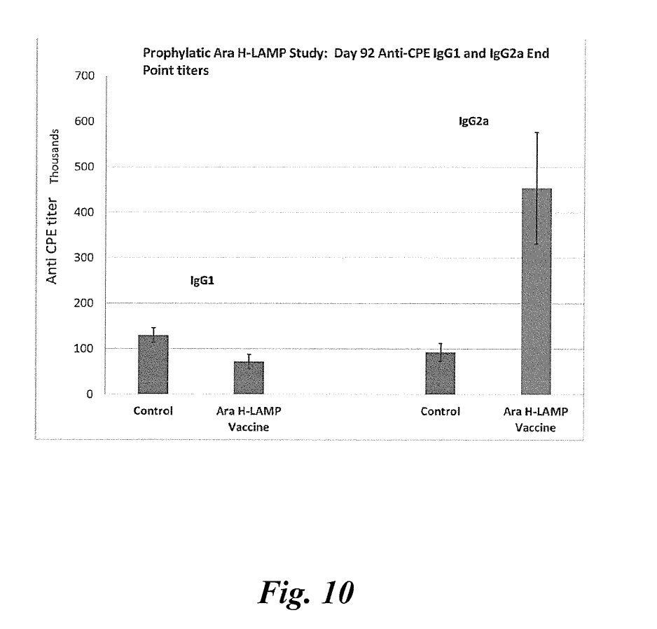

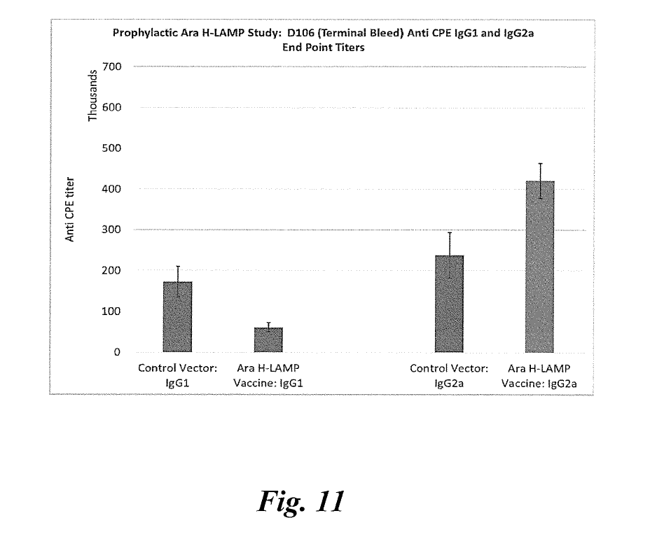

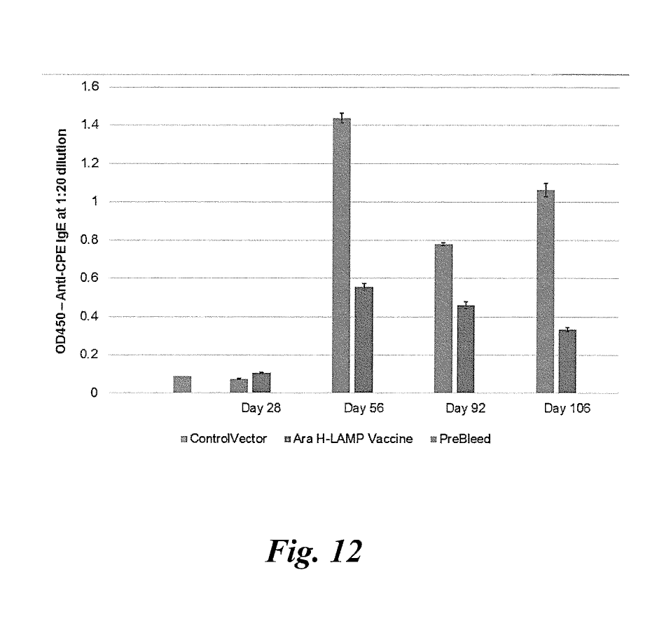

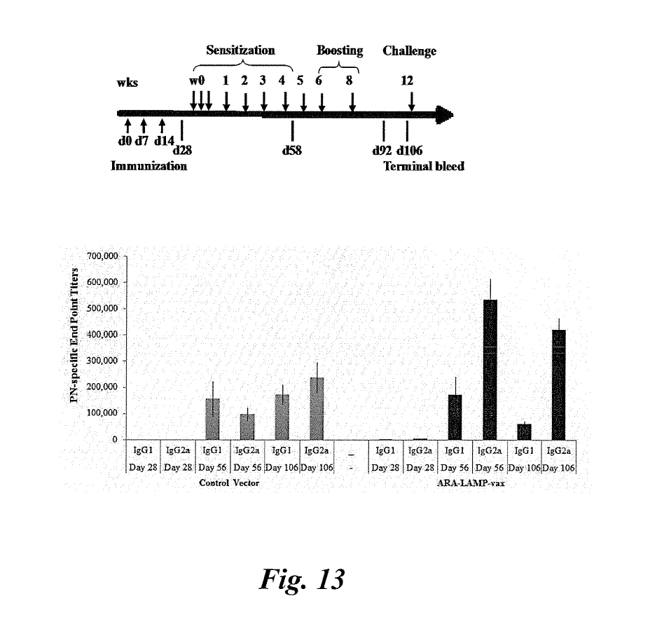

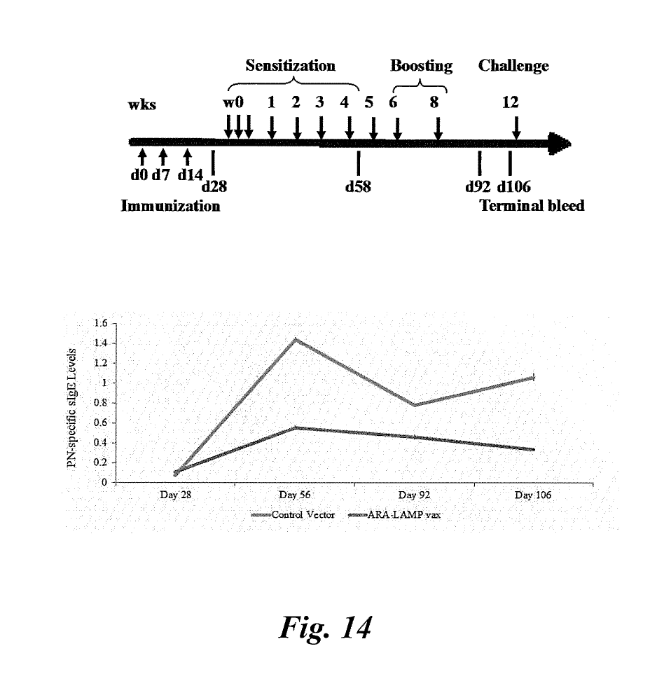

The representative protocol shown in FIG. 7 was used for further prophylactic studies. Mice were immunized on days 0, 7, and 14 with the single multivalent Ara H-LAMP DNA vaccine (weeks-3, -2, -1). IgG1 and IgG2a antibody levels were measured after immunization and IgG2a levels were found to be significantly higher with the single multivalent vaccine as compared to the control vector (FIG. 8). Mice were then sensitized with peanut paste (PN) and cholera toxin (CT) three times initially at week 0 and then weekly through week 5, followed by two boostings at weeks 6 and 8. IgG2a antibody levels at day 58 (week 5) were significantly higher with the multivalent vaccine as compared to the control vector (FIG. 9). After the two boostings, IgG2a antibody levels at day 92 were also significantly higher with the single multivalent vaccine as compared to the control vector (FIG. 10). This significant difference in IgG2a antibody levels continued after the challenge with peanut paste at week 12 (FIG. 11). Attenuation of the IgE response was seen throughout with the single multivalent vaccine (FIG. 12) supporting the prophylactic mechanism of the multivalent vaccine. FIGS. 13 and 14 show summaries of the prophylactic studies.

Interestingly, cell transfection with the single multivalent plasmid showed that all Ara h allergens produced fusion proteins in similar quantity to plasmids encoding a single allergen. Thus modifying the length of identity of the linker sequences may improve immunogenicity to all allergens. These results illustrate that multivalent allergy plasmids can successfully be designed that have excellent in vitro expression and broad immunogenicity. Further, these results show that the presently disclosed DNA vaccines can be used to prophylactically treat a subject for peanut allergies.

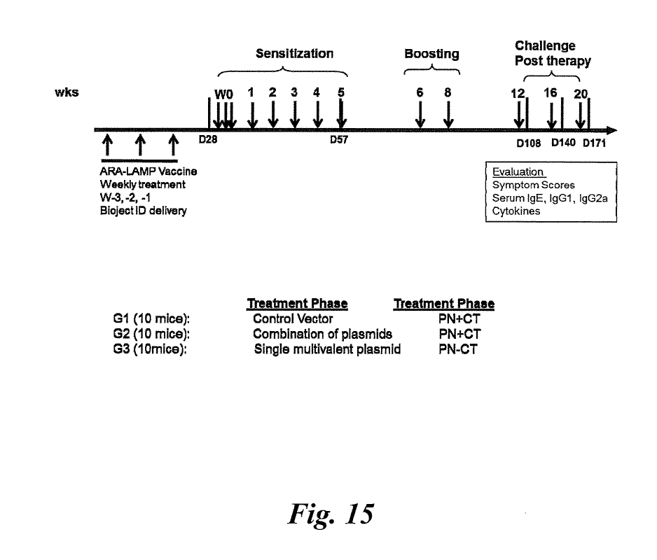

Further prophylactic studies comparing a combination of AraH1-LAMP, AraH2-LAMP, and AraH3del-LAMP plasmids versus a single multivalent AraH1/H2/H3-LAMP plasmid using Bioject ID delivery were conducted in accordance with the representative protocol shown in FIG. 15. Five week old female C3H/HeJmice (N=10 mice/group) were immunized on day 0, 7, and 14 (wk-3, -2, -1) with either a combination of Ara H1-LAMP, Ara H2-LAMP, and Ara H3del-LAMP plasmids (50 μg each) or a single multivalent AraH1/H2/H3 LAMP DNA plasmid (50 ug). Mice were then sensitized with 10 mg Peanut paste (PN)+20 μg CT, intragastrically (i.g.) three times initially at week (W) 0 and then weekly through W5 followed by two hoostings with 50 mg PN+20 ug CT, i.g. at W6 and W8. Mice which received the Control Vector (50 ug) were included as a control. Mice were then challenged with 200 mg PN, i.g., at W12, W16, and W20. Immunological responses were determined.

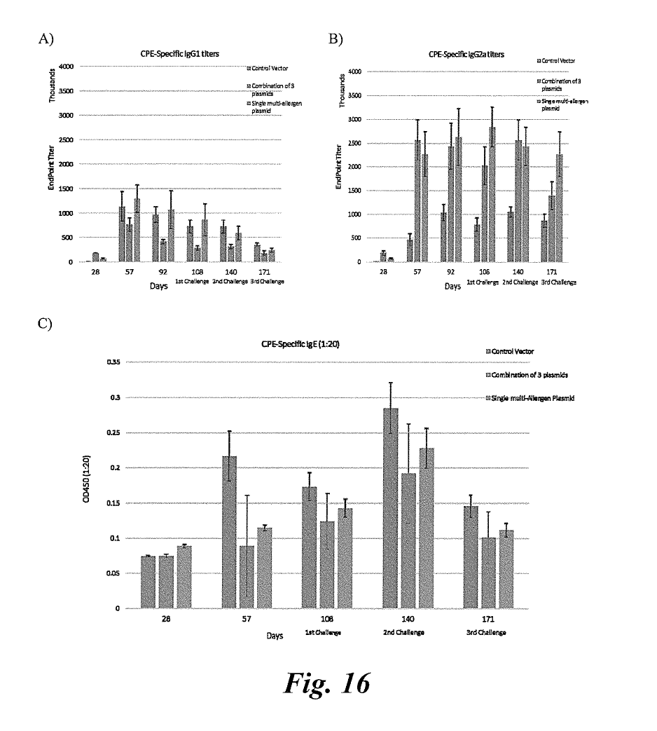

The results in FIG. 16 show that both the combination of single AraH1-LAMP-vax, AraH2-LAMP-vax, and Ara-H3del-LAMP-vax plasmids and the single multivalent Ara H1/H2/H3-LAMP plasmid induced a strong IgG2a response when delivered by intradermal injection (ID) via the Bioject B2000 needle-free device. The single multivalent Ara H1/H2/H3 LAMP plasmid, however, induced a stronger antibody response as a whole and also suppressed peanut-specific IgE.

### Example 3

### ARA-LAMP Therapeutic Studies

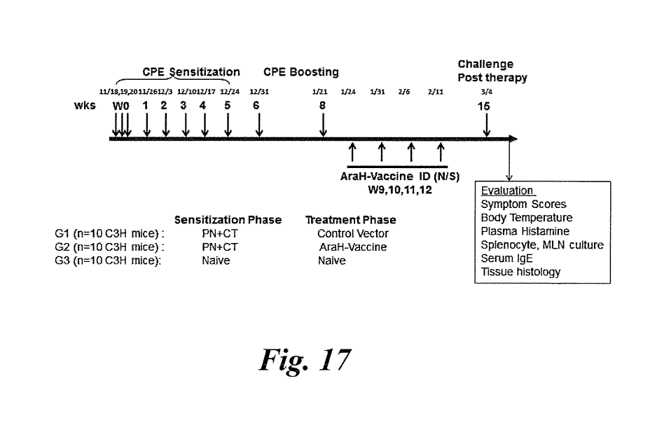

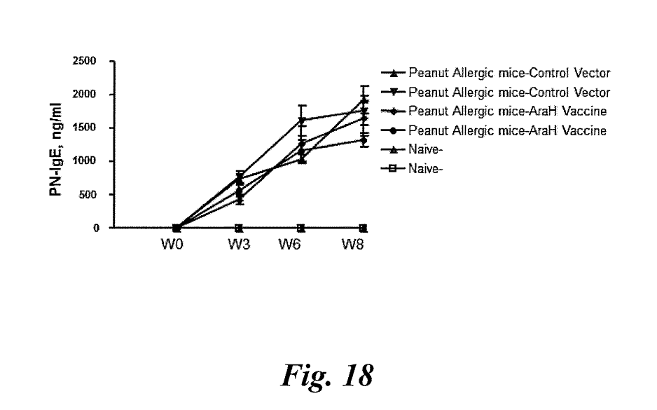

Experiments were also performed to determine the ability of the presently disclosed DNA vaccines to provide therapeutic treatment. A representative protocol is shown in FIG. 17 in which mice were first sensitized using peanut paste and cholera toxin and then were treated with the presently disclosed ARA-LAMP-vax three-plasmid (AraH1-LAMP, AraH2-LAMP, and Ara-H3del-LAMP, prepared in accordance with Example 1) composition. FIG. 18 shows the IgE antibody levels during the weeks prior to vaccine treatment with ARA-LAMP-vax, the three plasmid mix, each plasmid encoding a single peanut allergen.

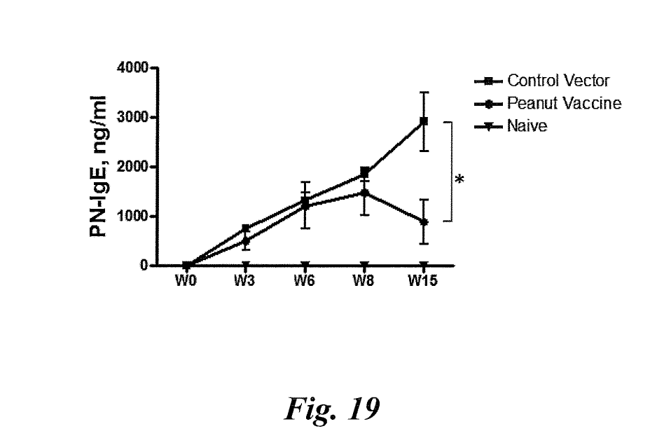

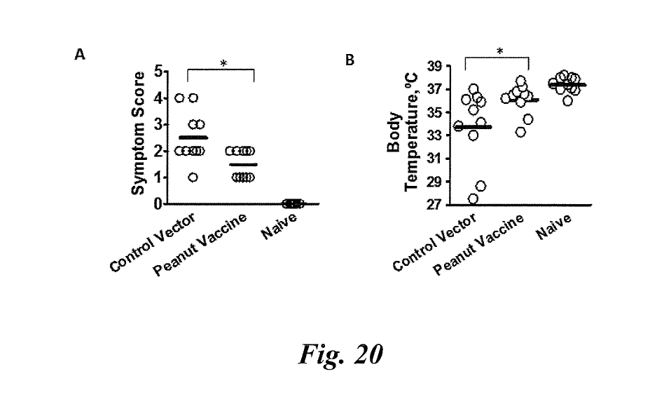

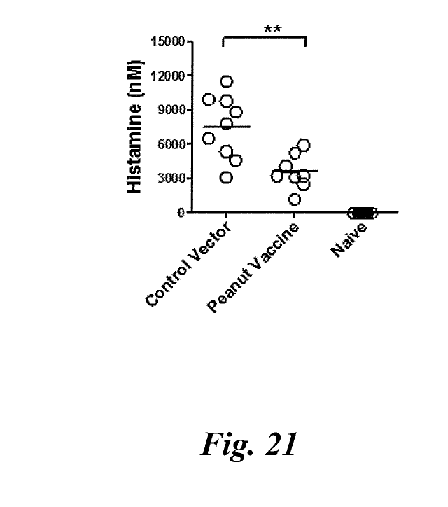

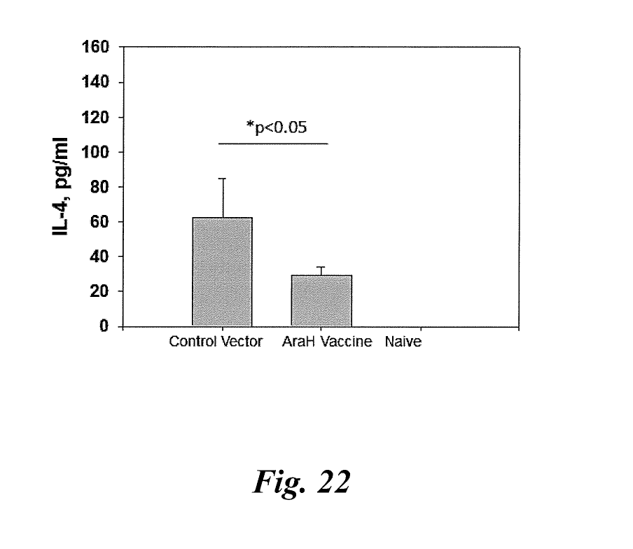

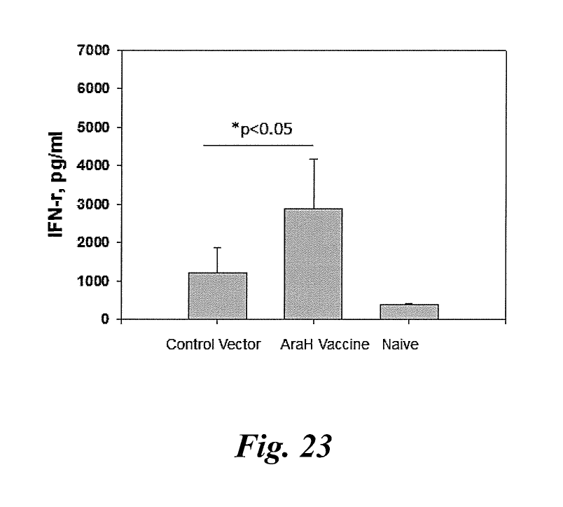

After vaccine treatment, IgE antibody levels at week 15 decreased when the multivalent DNA vaccine was used (FIG. 19). The anaphylaxis challenge results at week 15 (symptom scores, FIG. 20, Panel A; body temperature, FIG. 20, Panel B; FIG. 21) showed that administration of the ARA-LAMP-vax three-plasmid composition resulted in less severe symptoms and less plasma histamine levels as compared to the control vector. In addition, less of the pro-allergic cytokine, IL-4, was found with administration of the ARA-LAMP-vax three-plasmid composition (FIG. 22) whereas levels of IFN-γ were elevated (FIG. 23) relative to control vector. These results show that the presently disclosed DNA vaccines can be used for therapeutic treatment.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

### SEQUENCE LISTING

SEQ ID NO: 1 - AraH-LAMP (or AraH1-H2-H3-LAMP)
The amino acid sequence of the coding region for the Ara
H1/H2/H3 polyprotein chimeric construct, as follows:
SIGNAL: (1) . . . (27)
N-LAMP: (28) . . . (380)
AraH1: (383) . . . (983)
AraH2: (988) . . . (1138)
AraH3: (1143) . . . (1634)
TM/CYTO: (1637) . . . (1672)
Met Ala Pro Arg Ser Ala Arg Arg Pro Leu Leu Leu Leu Leu Leu Leu
Leu Leu Leu Gly Leu Met His Cys Ala Ser Ala Ala Met Phe Met Val
Lys Asn Gly Asn Gly Thr Ala Cys Ile Met Ala Asn Phe Ser Ala Ala
Phe Ser Val Asn Tyr Asp Thr Lys Ser Gly Pro Lys Asn Met Thr Leu
Asp Leu Pro Ser Asp Ala Thr Val Val Leu Asn Arg Ser Ser Cys Gly
Lys Glu Asn Thr Ser Asp Pro Ser Leu Val Ile Ala Phe Gly Arg Gly
His Thr Leu Thr Leu Asn Phe Thr Arg Asn Ala Thr Arg Tyr Ser Val
Gln Leu Met Ser Phe Val Tyr Asn Leu Ser Asp Thr His Leu Phe Pro
Asn Ala Ser Ser Lys Glu Ile Lys Thr Val Glu Ser Ile Thr Asp Ile
Arg Ala Asp Ile Asp Lys Lys Tyr Arg Cys Val Ser Gly Thr Gln Val
His Met Asn Asn Val Thr Val Thr Leu His Asp Ala Thr Ile Gln Ala
Tyr Leu Ser Asn Ser Ser Phe Ser Arg Gly Glu Thr Arg Cys Glu Gln
Asp Arg Pro Ser Pro Thr Thr Ala Pro Pro Ala Pro Pro Ser Pro Ser
Pro Ser Pro Val Pro Lys Ser Pro Ser Val Asp Lys Tyr Asn Val Ser
Gly Thr Asn Gly Thr Cys Leu Leu Ala Ser Met Gly Leu Gln Leu Asn
Leu Thr Tyr Glu Arg Lys Asp Asn Thr Thr Val Thr Arg Leu Leu Asn
Ile Asn Pro Asn Lys Thr Ser Ala Ser Gly Ser Cys Gly Ala His Leu
Val Thr Leu Glu Leu His Ser Glu Gly Thr Thr Val Leu Leu Phe Gln
Phe Gly Met Asn Ala Ser Ser Ser Arg Phe Phe Leu Gln Gly Ile Gln
Leu Asn Thr Ile Leu Pro Asp Ala Arg Asp Pro Ala Phe Lys Ala Ala
Asn Gly Ser Leu Arg Ala Leu Gln Ala Thr Val Gly Asn Ser Tyr Lys
Cys Asn Ala Glu Glu His Val Arg Val Thr Lys Ala Phe Ser Val Asn
Ile Phe Lys Val Trp Val Gln Ala Phe Lys Val Glu Gly Gly Gln Phe
Gly Ser Val Glu Glu Cys Leu Leu Asp Glu Asn Ser Leu Glu Lys Ser
Ser Pro Tyr Gln Lys Lys Thr Glu Asn Pro Cys Ala Gln Arg Cys Leu
Glu Ser Cys Gln Gln Glu Pro Asp Asp Leu Lys Glu Lys Ala Cys Glu
Ser Arg Cys Thr Lys Leu Glu Tyr Asp Pro Arg Cys Val Tyr Asp Pro
Arg Gly His Thr Gly Thr Thr Asn Gln Arg Ser Pro Pro Gly Glu Arg
Thr Arg Gly Arg Gln Pro Gly Asp Tyr Asp Asp Asp Arg Arg Gln Pro
Arg Arg Glu Glu Gly Gly Arg Trp Gly Pro Ala Gly Pro Arg Glu Arg
Glu Arg Glu Glu Asp Trp Arg Gln Pro Arg Glu Asp Trp Arg Arg Pro
Ser His Gln Gln Pro Arg Lys Ile Arg Pro Glu Gly Arg Glu Gly Glu
Gln Glu Trp Gly Thr Pro Gly Ser His Val Arg Glu Glu Thr Ser Arg
Asn Asn Pro Phe Tyr Phe Pro Ser Arg Arg Phe Ser Thr Arg Tyr Gly
Asn Gln Asn Gly Arg Ile Arg Val Leu Gln Arg Phe Asp Gln Arg Ser
Arg Gln Phe Gln Asn Leu Gln Asn His Arg Ile Val Glu Ile Glu Ala
Lys Pro Asn Thr Leu Val Leu Pro Lys His Ala Asp Ala Asp Asn Ile
Leu Val Ile Gln Gln Gly Gln Ala Thr Val Thr Val Ala Asn Gly Asn
Asn Arg Lys Ser Phe Asn Leu Asp Glu Gly His Ala Leu Arg Ile Pro
Ser Gly Phe Ile Ser Tyr Ile Leu Asn Arg His Asp Asn Gln Asn Leu
Arg Val Ala Lys Ile Ser Met Pro Val Asn Thr Pro Gly Gln Phe Glu
Asp Phe Phe Pro Ala Ser Ser Arg Asp Gln Ser Ser Tyr Leu Gln Gly
Phe Ser Arg Asn Thr Leu Glu Ala Ala Phe Asn Ala Glu Phe Asn Glu
Ile Arg Arg Val Leu Leu Glu Glu Asn Ala Gly Gly Glu Gln Glu Glu
Arg Gly Gln Arg Arg Trp Ser Thr Arg Ser Ser Glu Asn Asn Glu Gly
Val Ile Val Lys Val Ser Lys Glu His Val Glu Glu Leu Thr Lys His
Ala Lys Ser Val Ser Lys Lys Gly Ser Glu Glu Glu Gly Asp Ile Thr
Asn Pro Ile Asn Leu Arg Glu Gly Glu Pro Asp Leu Ser Asn Asn Phe
Gly Lys Leu Phe Glu Val Lys Pro Asp Lys Lys Asn Pro Gln Leu Gln
Asp Leu Asp Met Met Leu Thr Cys Val Glu Ile Lys Glu Gly Ala Leu
Met Leu Pro His Phe Asn Ser Lys Ala Met Val Ile Val Val Val Asn
Lys Gly Thr Gly Asn Leu Glu Leu Val Ala Val Arg Lys Glu Gln Gln
Gln Arg Gly Arg Arg Glu Glu Glu Glu Asp Glu Asp Glu Glu Glu Glu
Gly Ser Asn Arg Glu Val Arg Arg Tyr Thr Ala Arg Leu Lys Glu Gly
Asp Val Phe Ile Met Pro Ala Ala His Pro Val Ala Ile Asn Ala Ser
Ser Glu Leu His Leu Leu Gly Phe Gly Ile Asn Ala Glu Asn Asn His
Arg Ile Phe Leu Ala Gly Asp Lys Asp Asn Val Ile Asp Gln Ile Glu
Lys Gln Ala Lys Asp Leu Ala Phe Pro Gly Ser Gly Glu Gln Val Glu
Lys Leu Ile Lys Asn Gln Lys Glu Ser His Phe Val Ser Ala Arg Pro
Gln Ser Gln Ser Gln Ser Pro Ser Ser Pro Glu Lys Glu Ser Pro Glu
Lys Glu Asp Gln Glu Glu Glu Asn Gln Gly Gly Lys Gly Pro Leu Leu
Ser Ile Leu Lys Ala Phe Asn Gly Gly Gly Gly Arg Gln Gln Trp Glu
Leu Gln Gly Asp Arg Arg Cys Gln Ser Gln Leu Glu Arg Ala Asn Leu
Arg Pro Cys Glu Gln His Leu Met Gln Lys Ile Gln Arg Asp Glu
Asp Ser Tyr Gly Arg Asp Pro Tyr Ser Pro Ser Gln Asp Pro Tyr
Ser Pro Ser Gln Asp Pro Asp Arg Arg Asp Pro Tyr Ser Pro Ser
Pro Tyr Asp Arg Arg Gly Ala Gly Ser Ser Gln His Gln Glu Arg
Cys Cys Asn Glu Leu Asn Glu Phe Glu Asn Asn Gln Arg Cys Met
Cys Glu Ala Leu Gln Gln Ile Met Glu Asn Gln Ser Asp Arg Leu
Gln Gly Arg Gln Gln Glu Gln Gln Phe Lys Arg Glu Leu Arg Asn
Leu Pro Gln Gln Cys Gly Leu Arg Ala Pro Gln Arg Cys Asp Leu
Glu Val Glu Ser Gly Gly Arg Asp Arg Tyr Gly Gly Gly Gly Val
Thr Phe Arg Gln Gly Gly Glu Glu Asn Glu Cys Gln Phe Gln Arg
Leu Asn Ala Gln Arg Pro Asp Asn Arg Ile Glu Ser Glu Gly Gly
Tyr Ile Glu Thr Trp Asn Pro Asn Asn Gln Glu Phe Gln Cys Ala
Gly Val Ala Leu Ser Arg Thr Val Leu Arg Arg Asn Ala Leu Arg
Arg Pro Phe Tyr Ser Asn Ala Pro Leu Glu Ile Tyr Val Gln Gln
Gly Ser Gly Tyr Phe Gly Leu Ile Phe Pro Gly Cys Pro Ser Thr
Tyr Glu Glu Pro Ala Gln Glu Gly Arg Arg Tyr Gln Ser Gln Lys
Pro Ser Arg Arg Phe Gln Val Gly Gln Asp Asp Pro Ser Gln Gln
Gln Gln Asp Ser His Gln Lys Val His Arg Phe Asp Glu Gly Asp
Leu Ile Ala Val Pro Thr Gly Val Ala Phe Trp Met Tyr Asn Asp
Glu Asp Thr Asp Val Val Thr Val Thr Leu Ser Asp Thr Ser Ser
Ile His Asn Gln Leu Asp Gln Phe Pro Arg Arg Phe Tyr Leu Ala
Gly Asn Gln Glu Gln Glu Phe Leu Arg Tyr Gln Gln Gln Gln Gly
Ser Arg Pro His Tyr Arg Gln Ile Ser Pro Arg Val Arg Gly Asp
Glu Gln Glu Asn Glu Gly Ser Asn Ile Phe Ser Gly Phe Ala Gln
Glu Phe Leu Gln His Ala Phe Gln Val Asp Arg Gln Thr Val Glu
Asn Leu Arg Gly Glu Asn Glu Arg Glu Glu Gln Gly Ala Ile Val
Thr Val Lys Gly Gly Leu Arg Ile Leu Ser Pro Asp Glu Glu Asp
Glu Ser Ser Arg Ser Pro Pro Asn Arg Arg Glu Glu Phe Asp Glu
Asp Arg Ser Arg Pro Gln Gln Arg Gly Lys Tyr Asp Glu Asn Arg
Arg Gly Tyr Lys Asn Gly Ile Glu Glu Thr Ile Cys Ser Ala Ser
Val Lys Lys Asn Leu Gly Arg Ser Ser Asn Pro Asp Ile Tyr Asn
Pro Gln Ala Gly Ser Leu Arg Ser Val Asn Glu Leu Asp Leu Pro
Ile Leu Gly Trp Leu Gly Leu Ser Ala Gln His Gly Thr Ile Tyr
Arg Asn Ala Met Phe Val Pro His Tyr Thr Leu Asn Ala His Thr
Ile Val Val Ala Leu Asn Gly Arg Ala His Val Gln Val Val Asp
Ser Asn Gly Asn Arg Val Tyr Asp Glu Glu Leu Gln Glu Gly His
Val Leu Val Val Pro Gln Asn Phe Ala Val Ala Ala Lys Ala Gln
Ser Glu Asn Tyr Glu Tyr Leu Ala Phe Lys Thr Asp Ser Arg Pro
Ser Ile Ala Asn Gln Ala Gly Glu Asn Ser Ile Ile Asp Asn Leu
Pro Glu Glu Val Val Ala Asn Ser Tyr Arg Leu Pro Arg Glu Gln
Ala Arg Gln Leu Lys Asn Asn Asn Pro Phe Lys Phe Phe Val Pro
Pro Phe Asp His Gln Ser Met Arg Glu Val Ala Glu Phe Thr Leu
Ile Pro Ile Ala Val Gly Gly Ala Leu Ala Gly Leu Val Leu Ile
Val Leu Ile Ala Tyr Leu Val Gly Arg Lys Arg Ser His Ala Gly
Tyr Gln Thr Ile
SEQ ID NO: 2 - Ara H1
The amino acid sequence of the coding region for the Ara H1
protein without the signal sequence (in the Ara H1/H2/H3
polyprotein chimeric construct, AraH-LAMP, and in the
individual Ara H1 construct, AraH1-LAMP), as follows:
KSSPYQKKTENPCAQRCLQSCQQEPDDLKQKACESRCTKLEYDPRCVYDPRGHT
GTTNQRSPPGERTRGRQPGDYDDDRRQPRREEGGRWGPAGPREREREEDWRQP
REDWRRPSHQQPRKIRPEGREGEQEWGTPGSHVREETSRNNPFYFPSRRFSTRYG
NQNGRIRVLQRFDQRSRQFQNLQNHRIVQIEAKPNTLVLPKHADADNILVIQQGQ
ATVTVANGNNRKSFNLDEGHALRIPSGFISYILNRHDNQNLAVAKISMINNTPGQ
FEDFFPASSRDQSSYLQGFSRNTLEAAFNAEFNEIRRVLLEENAGGEQEERGQRR
WSTRSSENNEGVIVKVSKEHVEELTKHAKSVSKKGSEEEGDITNPINLREGEPDLS
NNFGKLFEVKPDKKNPQLQDLDMMLTCVEIKEGALMLPHFNSKAMVIVVVNKG
TGNLELVAVRKEQQQRGRREEEEDEDEEEEGSNREVRRYTARLKEGDVFIMPAA
HPVAINASSELHLLGEGINAENNHRIFLAGDKDNVIDQIEKQAKDLAFPGSGEQVE
KLIKNQKESHFVSARPQSQSQSPSSPEKESPEKEDQEEENQGGKGPLLSILKAFN
SEQ ID NO: 3 - Ara H2
The amino acid sequence of the coding region for the
Ara H2 protein without the native signal sequence (in
the Ara H1/H2/H3 polyprotein chimeric construct,
AraH-LAMP, and in the individual Ara H2 construct,
AraH2-LAMP), as follows:
RQQWELQGDRRCQSQLERANLRPCEQHLMQKIQRDEDSYGRDPYSPSQDPYSPS
QDPDRRDPYSPSPYDRRGAGSSQHQERCCNELNEFENNQRCMCEALQQIMENQS
DRLQGRQQEQQFKRELRNLPQQCGLRAPQRCDLEVESGGRDRY
SEQ ID NO: 4 - Ara H3 in polyprotein chimeric
construct, AraH-LAMP (or AraH1-H2-H3-LAMP)
The amino acid sequence of the coding region for the
Ara H3 protein without the native signal sequence in
the Ara H1/H2/H3 polyprotein chimeric construct, as
follows:
VTFRQGGEENECQFQRLNAQRPDNRIESEGGYIETWNPNNQEFQCAGVALSRTV
LRRNALRRPFYSNAPLEIYVQQGSGYFGLIFPGCPSTYEEPAQEGRRYQSQKPSRR
FQVGQDDPSQQQQDSHQKVHRFDEGDLIAVPTGVAFWMYNDEDTDVVTVTLSD
TSSIHNQLDQFPRRFYLAGNQEQEFLRYQQQQGSRPHYRQISPRVRGDEQENEGS
NIFSGFAQEFLQHAFQVDRQTVENLRGENEREEQGAIVTVKGGLRILSPDEEDESS
RSPPNRREEFDEDRSRPQQRGKYDENRRGYKNGIEETICSASVKKNLGRSSNPDI
YNPQAGSLRSVNELDLPILGWLGLSAQHGTIYRNAMFVPHYTLNAHTIVVALNG
RAHVQVVDSNGNRVYDEELQEGHVLVVPQNFAVAAKAQSENYEYLAFKTDSRP
SIANQAGENSIIDNLPEEVVANSYRLPREQARQLKNNNPFKFFVPPFDHQSMREV
A
SEQ ID NO: 5 - Ara H3del in the individual Ara
H3del construct, AraH3del LAMP
The amino acid sequence of the coding region for
the Ara H3del protein (truncated version that was
designed to avoid splicing sites; le = Xho, ef =
EcoRI) in the Ara H3del individual construct, as
follows:
VTFRQGGEENECQFQRLNAQRPDNRIESEGGYIETWNPNNQEFQCAGVALSRTV
LRRNALRRPFYSNAPLEIYVQQGSGYFGLIFPGCPSTYEEPAQEGRRYQSQKPSRR
FQVGQDDPSQQQQDSHQKVHRFDEGDLIAVPTGVAFWMYNDEDTDVVTVTLS
DTSSIHNQLDQFPRRFYLAGNQEQEFLRYQQQQGSRPHYRQISPRVRGDEQENEG
SNIFSGFAQEFLQHAFQVDRQTVENLRGENEREEQGAIVTVKGGLRILSPDEEDES
SRSPPNRREEFDEDRSRPQQRGKYDENRRGYKN
SEQ ID NO: 6 - AraH3del LAMP
The amino acid sequence of the coding region for
the Ara H3 LAMP fusion protein (LAMP is in bold;
flanking XhoI (LE) and EcoRI (EF) sites are
uppercase and underlined), as follows:
MAPRSARRPLLLLLLLLLLGLMHCASAAMFMVKNGNGTACIMANFSAAFSV
NYDTKSGPKNMTLDLPSDATVVLNRSSCGKENTSDPSLVIAFGRGHTLTLNF
TRNATRYSVQLMSFVYNLSDTHLFPNASSKEIKTVESITDIRADIDKKYRCVS
GTQVHMNNVTVTLHDATIQAYLSNSSFSRGETRCEQDRPSPTTAPPAPPSPSP
SPVPKSPSVDKYNVSGTNGTCLLASMGLQLNLTYERKDNTTVTRLLNINPNK
TSASGSCGAHLVTLELHSEGTTVLLFQFGMNASSSRFFLQGIQLNTILPDARD
PAFKAANGSLRALQATVGNSYKCNAEHIVRVTKAFSVNIFKVWVQAFKVEG
GQFGSVEECLLDENSLEVTFRQGGEENECQFQRLNAQRPDNRIESEGGYIETWN
PNNQEFQCAGVALSRTVLRRNALRRPFYSNAPLEIYVQQGSGYFGLIFPGCPSTY
EEPAQEGRRYQSQKPSRRFQVGQDDPSQQQQDSHQKVHRFDEGDLIAVPTGVAF
WMYNDEDTDVVTVTLSDTSSIHNQLDQFPRRFYLAGNQEQEFLRYQQQQGSRP
HYRQISPRVRGDEQENEGSNIFSGFAQEFLQHAFQVDRQTVENLRGENEREEQGA
IVTVKGGLRILSPDEEDESSRSPPNRREEFDEDRSRPQQRGKYDENRRGYKNEFT
LIPIAVGGALAGLVLIVLIAYLVGRKRSHAGYQTI*
SEQ ID NO: 7 - AraH2 LAMP
The amino acid sequence of the coding region for
the Ara H2 LAMP fusion protein (LAMP is in bold;
flanking XhoI (LE) and EcoRI (EF) sites are
uppercase and underlined), as follows:
MAPRSARRPLLLLLLLLLLGLMHCASAAMFMVKNGNGTACIMANFSAAFSV
NYDTKSGPKNMTLDLPSDATVVLNRSSCGKENTSDPSLVIAFGRGHTLTLNF
TRNATRYSVQLMSFVYNLSDTHLFPNASSKEIKTVESITDIRADIDKKYRCVS
GTQVHMNNVTVTLHDATIQAYLSNSSFSRGETRCEQDRPSPTTAPPAPPSPSP
SPVPKSPSVDKYNVSGTNGTCLLASMGLQLNLTYERKDNTTVTRLLNINPNK
TSASGSCGAHLVTLELHSEGTTVLLFQFGMNASSSRFFLQGIQLNTILPDARD
PAFKAANGSLRALQATVGNSYKCNAEEHVRVTKAFSVNIFKVWVQAFKVEG
GQFGSVEECLLDENSLERQQWELQGURRCQSQLERANLRPCEQHLMQKIQRDE
DSYGRDPYSPSQDPYSPSQDPDRRDPYSPSPYDRRGAGSSQHQERCCNELNEFEN
NQRCMCEALQQIMENQSDRLQGRQQEQQFKRELRNLPQQCGLRAPQRCDLEVE
SGGRDRYEFTLIPIAVGGALAGLVLIVLIAYLVGRKRSHAGYQTI*
SEQ ID NO: 8 - AraH1 LAMP
The amino acid sequence of the coding region for
the Ara H1 LAMP fusion protein (LAMP is in bold;
flanking XhoI (LE) and EcoRI (EF) sites are
uppercase and underlined), as follows:
MAPRSARRPLLLLLIALLLGLMHCASAAMFMVKNGNGTACIMANFSAAESV
NYDTKSGPKNMTLDLPSDATVVLNRSSCGKENTSDPSLVIAFGRGHTLTLNF
TRNATRYSVQLMSFVYNLSDTHLFPNASSKEIKTVESITDIRADIDKKYRCVS
GTQVHMNNVTVTLHDATIQAYLSNSSFSRGETRCEQDRPSPTTAPPAPPSPSP
SPVPKSPSVDKYNVSGTNGTCLLASMGLQLNLTYERKDNTTVTRLLNINPNK
TSASGSCGAHLVTLELHSEGTTVLLFQFGMNASSSRFFLQGIQLNTILPDARD
PAFKAANGSLRALQATVGNSYKCNAEEHVRVTKAFSVNIFKVWVQAFKVEG
GQFGSVEECLLDENSLEKSSPYQKKTENPCAQRCLQSCQQEPDDLKQKACESR
CTKLEYDPRCVYDPRGHTGTTNQRSPPGERTRGRQPGDYDDDRRQPRREEGGR
WGPAGPREREREEDWRQPREDWRRPSHQQPRKIRPEGREGEQEWGTPGSHVRE
ETSRNNPFYFPSRRESTRYGNQNGRIRVLQRFDQRSRQFQNLQNHRIVQIEAKPNT
LVLPKHADADNILVIQQGQATVTVANGNNRKSFNLDEGHALRIPSGFISYILNRH
DNQNLRVAKISMPVNTPGQFEDFFPASSRDQSSYLQGFSRNTLEAAFNAEFNEIR
RVLLEENAGGEQEERGQRRWSTRSSENNEGVIVKVSKEHVEELTKHAKSVSKKG
SEEEGDITNPINLREGEPDLSNNFGKLFEVKPDKKNPQLQDLDMMLTCVEIKEGA
LMLPHFNSKAMVIVVVNKGTGNLELVAVRKEQQQRGRREEEEDEDEEEEGSNR
EVRRYTARLKEGDVFIMPAAHPVAINASSELHLLGFGINAENNHRIFLAGDKDNV
IDQIEKQAKDLAFPGSGEQVEKLIKNQKESHFVSARPQSQSQSPSSPEKESPEKED
QEEENQGGKGPLLSILKAFNEFTLIPIAVGGALAGLVLIVLIAYLVGRKRSHAG
YQTI*
SEQ ID NO: 9 - Deleted Ara H3 region
The amino acid sequence of Ara H3 not included in the
individual Ara H3del construct, as follows:
GIEETICSASVKKNLGRSSNPDIYNPQAGSLRSVNELDLPILGWLGLSAQHGTIYR
NAMFVPHYTLNAHTIVVALNGRAHVQVVDSNGNRVYDEELQEGHVLVVPQNF
AVAAKAQSENYEYLAFKTDSRPSIANQAGENSIIDNLPEEVVANSYRLPREQARQ
LKNNNPFKFFVPPFDHQSMREVA
SEQ ID NO: 10 - LAMP2 Nucleotide sequence
SIGNAL: (1) . . . (84)
STABILIZING: (85) . . . (1125)
TM/CYTO: (1126) . . . (1227)
atggtgtgcttccgcctcttcccggttccgggctcagggctcgttctggtctgcctagtcctgggagctgtgcggtcttatgcattg
gaacttaatttgacagattcagaaaatgccacttgcctttatgcaaaatggcagatgaatttcacagttcgctatgaaactacaaata
aaacttataaaactgtaaccatttcagaccatggcactgtgacatataatggaagcatttgtggggatgatcagaatggtcccaaaa
tagcagtgcagttcggacctggcttttcctggattgcgaattttaccaaggcagcatctacttattcaattgacagcgtctcattttcct
acaacactggtgataacacaacatttcctgatgctgaagataaaggaattcttactgttgatgaacttttggccatcagaattccattg
aatgacctttttagatgcaatagtttatcaactttggaaaagaatgatgttgtccaacactactgggatgttcttgtacaagcttttgtcc
aaaatggcacagtgagcacaaatgagttcctgtgtgataaagacaaaacttcaacagtggcacccaccatacacaccactgtgc
catctcctactacaacacctactccaaaggaaaaaccagaagctggaacctattcagttaataatggcaatgatacttgtctgctgg
ctaccatggggctgcagctgaacatcactcaggataaggttgcttcagttattaacatcaaccccaatacaactcactccacaggc
agctgccgttctcacactgctctacttagactcaatagcagcaccattaagtatctagactttgtctttgctgtgaaaaatgaaaacc
gattttatctgaaggaagtgaacatcagcatgtatttggttaatggctccgttttcagcattgcaaataacaatctcagctactggatg
cccccaagttcttatatgtgcaacaaagagcagactgtttcagtgtctggagcatttcagataaatacctttgatctaagggttcagc
ctttcaatgtgacacaaggaaagtattctacagctcaagactgcagtgcagatgacgacaacttccttgtgcccatagcggtggg
agctgccttggcaggagtacttattctagtgttgctggcttattttattggtctcaagcaccatcatgctggatatgagcaattttag
SEQ ID NO: 11 - LAMP-3 (DC-LAMP) Nucleotide Sequence
SIGNAL: (1) . . . (81)
STABILIZING: (82) . . . (1143)
TM/CYTO: (1144) . . . (1248)
atgccccggcagctcagcgcggcggccgcgctcttcgcgtccctggccgtaattttgcacgatggcagtcaaatgagagcaaa
agcatttccagaaaccagagattattctcaacctactgcagcagcaacagtacaggacataaaaaaacctgtccagcaaccagct
aagcaagcacctcaccaaactttagcagcaagattcatggatggtcatatcacctttcaaacagcggccacagtaaaaattccaa
caactaccccagcgactacaaaaaacactgcaaccaccagcccaattacctacaccctggtcacaacccaggccacacccaac
aactcacacacagctcctccagttactgaagttacagtcggccctagcttagccccttattcactgccacccaccatcaccccacc
agctcatacaactggaaccagttcatcaaccgtcagccacacaactgggaacaccactcaacccagtaaccagaccacccttcc
agcaactttatcgatagcactgcacaaaagcacaaccggtcagaagcctgttcaacccacccatgccccaggaacaacggcag
ctgcccacaataccacccgcacagctgcacctgcctccacggttcctgggcccacccttgcacctcagccatcgtcagtcaaga
ctggaatttatcaggttctaaacggaagcagactctgtataaaagcagagatggggatacagctgattgttcaagacaaggagtc
ggttttttcacctcggagatacttcaacatcgaccccaacgcaacgcaagcctctgggaactgtggcacccgaaaatccaacctt
ctgttgaattttcagggcggatttgtgaatctcacatttaccaaggatgaagaatcatattatatcagtgaagtgggagcctatttgac
cgtctcagatccagagacaatttaccaaggaatcaaacatgcggtggtgatgttccagacagcagtcgggcattccttcaagtgc
gtgagtgaacagagcctccagttgtcagcccacctgcaggtgaaaacaaccgatgtccaacttcaagcctttgattttgaagatga
ccactttggaaatgtggatgagtgctcgtctgactacacaattgtgcttcctgtgattggggccatcgtggttggtctctgccttatgg
gtatgggtgtctataaaatccgcctaaggtgtcaatcatctggataccagagaatc
SEQ ID NO: 12 - ENDOLYN Nucleotide Sequence
SIGNAL: (1) . . . (72)
STABILIZING: (73) . . . (486)
TM/CYTO: (487) . . . (594)
atgtcgcggctctcccgctcactgctttgggccgccacctgcctgggcgtgctctgcgtgctgtccgcggacaagaacacgacc
cagcacccgaacgtgacgactttagcgcccatctccaacgtaacctcggcgccggtgacgtccctcccgctggtcaccactcc
ggcaccagaaacctgtgaaggtcgaaacagctgcgtttcctgttttaatgttagcgttgttaatactacctgcttttggatagaatgta
aagatgagagctattgttcacataactcaacagttagtgattgtcaagtggggaacacgacagacttctgttccgtttccacggcca
ctccagtgccaacagccaattctacagctaaacccacagttcagccctccccttctacaacttccaagacagttactacatcaggt
acaacaaataacactgtgactccaacctcacaacctgtgcgaaagtctacctttgatgcagccagtttcattggaggaattgtcctg
gtcttgggtgtgcaggctgtaattttctttctttataaattctgcaaatctaaagaacgaaattaccacactctgtaa
SEQ ID NO: 13 - LIMP II Nucleotide Sequence
SIGNAL: (13) . . . (81)
STABILIZING: (82) . . . (1299)
TM/CYTO: (1300) . . . (1434)
atgggccgatgctgcttctacacggcggggacgttgtccctgctcctgctggtgaccagcgtcacgctgctggtggcccgggtc
ttccagaaggctgtagaccagagtatcgagaagaaaattgtgttaaggaatggtactgaggcatttgactcctgggagaagcccc
ctctgcctgtgtatactcagttctatttcttcaatgtcaccaatccagaggagatcctcagaggggagacccctcgggtggaagaa
gtggggccatacacctacagggaactcagaaacaaagcaaatattcaatttggagataatggaacaacaatatctgctgttagca
acaaggcctatgtttttgaacgagaccaatctgttggagaccctaaaattgacttaattagaacattaaatattcctgtattgactgtca
tagagtggtcccaggtgcacttcctcagggagatcatcgaggccatgttgaaagcctatcagcagaagctctttgtgactcacac
agttgacgaattgctctggggctacaaagatgaaatcttgtcccttatccatgttttcaggcccgatatctctccctattttggcctatt
ctatgagaaaaatgggactaatgatggagactatgtttttctaactggagaagacagttaccttaactttacaaaaattgtggaatgg
aatgggaaaacgtcacttgactggtggataacagacaagtgcaatatgattaatggaacagatggagattcttttcacccactaat
aaccaaagatgaggtcctttatgtcttcccatctgacttttgcaggtcagtgtatattactttcagtgactatgagagtgtacagggac
tgcctgcctttcggtataaagttcctgcagaaatattagccaatacgtcagacaatgccggcttctgtatacctgagggaaactgcc
tgggctcaggagttctgaatgtcagcatctgcaagaatggtgcacccatcattatgtctttcccacacttttaccaagcagatgaga
ggtttgtttctgccatagaaggcatgcacccaaatcaggaagaccatgagacatttgtggacattaatcctttgactggaataatcct
aaaagcagccaagaggttccaaatcaacatttatgtcaaaaaattagatgactttgttgaaacgggagacattagaaccatggtttt
cccagtgatgtacctcaatgagagtgttcacattgataaagagacggcgagtcgactgaagtctatgattaacactactttgatcat
caccaacataccctacatcatcatggcgctgggtgtgttctttggtttggtttttacctggcttgcatgcaaaggacagggatccatg
gatgagggaacagcggatgaaagagcacccctcattcgaacctag
SEQ ID NO: 14 - AraH1-AraH2-AraH3 Nucleotide sequence
AraH1: (1) . . . (1803)
LINKER: (1804) . . . (1815)
AraH2: (1816) . . . (2268)
LINKER: (2269) . . . (2280)
AraH3: (2281) . . . (3756)
aagtccagcccctaccagaagaaaaccgagaacccctgcgcccagcggtgcctgcagtcttgtcagcaggaacccgacgac
ctgaagcagaaggcctgcgagagccggtgcaccaagctggaatacgaccccagatgcgtgtacgaccctagaggccacacc
ggcaccaccaaccagagaagccctccaggcgagcggaccagaggcagacagcctggcgactacgacgacgacagacgg
cagcccagaagagaagagggcggcagatggggacctgccggccctagagagagagaacgcgaggaagattggagacag
cccagagaggactggcggaggccttctcaccagcagccccggaagatcagacccgagggcagagaaggcgagcaggaat
ggggcacacctggctctcacgtgcgcgaggaaaccagccggaacaaccccttctacttcccctcccggcggttcagcaccag
atacggcaaccagaacggccggatcagagtgctgcagagattcgaccagcggagccggcagttccagaacctgcagaacca
ccggatcgtgcagatcgaggccaagcccaacaccctggtgctgcccaaacacgccgacgccgacaacatcctcgtgatccag
cagggccaggccaccgtgacagtggccaacggcaacaacagaaagagcttcaacctggacgagggccacgccctgagaat
ccccagcggcttcatcagctacatcctgaacagacacgacaatcagaacctgagggtggccaagatcagcatgcccgtgaaca
cccctggccagttcgaggacttcttccccgcatcctcccgggaccagagcagctacctgcagggcttcagccggaataccctg
gaagccgccttcaacgccgagttcaacgagatcagacgggtgctgctggaagagaacgctggcggagagcaggaagaacg
gggccagagaagatggtccaccagaagcagcgagaacaacgagggcgtgatcgtgaaggtgtccaaagaacacgtggaag
aactgaccaagcacgccaagagcgtgtccaagaagggctccgaggaagagggggacatcaccaaccccatcaatctgagag
agggcgagcccgacctgagcaacaacttcggcaagctgttcgaagtgaagcccgacaagaagaacccccagctgcaggacc
tggacatgatgctgacctgcgtggaaatcaaagagggggccctgatgctgccacacttcaactccaaagccatggtcatcgtgg
tcgtgaacaagggcaccggcaacctggaactggtggccgtgcggaaagagcagcagcagagaggccgcagagaggaaga
agaggacgaggacgaagaagaagagggatccaaccgggaagtgcggcggtacaccgccagactgaaagaaggcgacgtg
ttcatcatgcctgccgcccaccccgtggccatcaatgcctctagcgagctgcatctgctgggcttcggcattaacgccgagaaca
atcaccggatctttctggccggcgacaaagacaacgtgatcgaccagatcgagaagcaggccaaggacctggcctttcccggc
tctggcgaacaagtggaaaagctgatcaagaaccagaaagaaagccacttcgtgtccgccagaccccagagccagtctcaga
gccctagctcccccgagaaagagtctcctgagaaagaggaccaggaagaggaaaaccagggcggcaagggccctctgctg
agcatcctgaaggccttcaatggcggcggaggcaggcagcagtgggaactgcagggcgacagaagatgccagtcccagctg
gaacgggccaacctgaggccttgcgagcagcacctgatgcagaaaatccagcgcgacgaggacagctacggccgggatcct
tacagccccagccaggacccttactcccctagccaggatcccgacagaagggacccctacagccctagcccctacgatagaa
gaggcgccggaagcagccagcaccaggaaagatgctgcaacgagctgaacgagtttgagaacaaccagcgctgcatgtgcg
aggccctgcagcagatcatggaaaatcagagcgaccggctgcagggacggcagcaggaacagcagttcaagagagagctg
cggaacctgccccagcagtgtggactgagagccccccagagatgcgacctggaagtggaaagcggcggcagagataggta
cggcggagggggcgtgaccttcagacagggcggagaagagaatgagtgccagtttcagcggctgaacgcccagaggcccg
acaacagaatcgagagcgagggcggctacatcgagacatggaaccccaacaaccaggaatttcagtgcgctggggtggccct
gagcaggaccgtgctgagaagaaatgccctgaggcggcccttctacagcaacgcccccctggaaatctacgtgcagcagggc
agcggctacttcggcctgatctttcccggatgcccctccacctatgaggaacccgctcaggaaggcagacggtatcagagcca
gaagcctagcagacggttccaagtgggccaggacgatcccagccaacagcagcaggactctcaccagaaggtgcaccgctt
cgacgagggcgacctgatcgctgtgccaaccggcgtggccttctggatgtacaacgacgaggataccgacgtcgtgaccgtg
accctgagcgacaccagctccatccacaaccagctggaccagttccccaggcggttttacctggccggcaatcaggaacagga
atttctgagataccagcagcagcagggctccagaccccactacagacagatcagccctagagtgcggggcgacgaacagga
aaatgagggcagcaacatcttctccggctttgcccaggaatttctgcagcacgccttccaggtggaccggcagaccgtggaaaa
cctgagaggcgagaacgagagagaggaacagggcgccatcgtgactgtgaagggcggcctgaggatcctgagccccgacg
aagaggatgagtcctctagaagcccccccaaccgccgggaagagttcgatgaggaccgcagcagacctcagcagcggggg
aagtacgacgagaacaggcggggctacaagaacggcatcgaggaaacaatctgcagcgccagcgtgaagaagaatctggg
ccggtccagcaaccccgacatctacaatccacaggccggcagcctgcggagcgtgaacgaactggatctgcccatcctggga
tggctgggcctgtctgcccagcacggcaccatctaccggaacgccatgttcgtgcctcactacaccctgaatgcccacaccatc
gtggtggctctgaacggccgcgcccacgtccaagtggtggacagcaacggcaatcgggtgtacgatgaagaactgcaggaa
ggacacgtcctggtggtgccccagaattttgccgtggccgccaaggcccagtccgagaactatgagtatctggccttcaagacc
gacagccggccctctatcgccaatcaagccggcgagaacagcatcatcgacaacctgcccgaggaagtggtggccaacagct
accggctgcctagagagcaggcccggcagctgaagaacaacaaccctttcaagttcttcgtgcccccattcgaccaccagagc
atgagagaggtggcc
SEQ ID NO: 15 - AraH1 Nucleotide sequence
aagtccagcccctaccagaagaaaaccgagaacccctgcgcccagcggtgcctgcagtcttgtcagcaggaacccgacgac
ctgaagcagaaggcctgcgagagccggtgcaccaagctggaatacgaccccagatgcgtgtacgaccctagaggccacacc
ggcaccaccaaccagagaagccctccaggcgagcggaccagaggcagacagcctggcgactacgacgacgacagacgg
cagcccagaagagaagagggcggcagatggggacctgccggccctagagagagagaacgcgaggaagattggagacag
cccagagaggactggcggaggccttctcaccagcagccccggaagatcagacccgagggcagagaaggcgagcaggaat
ggggcacacctggctctcacgtgcgcgaggaaaccagccggaacaaccccttctacttcccctcccggcggttcagcaccag
atacggcaaccagaacggccggatcagagtgctgcagagattcgaccagcggagccggcagttccagaacctgcagaacca
ccggatcgtgcagatcgaggccaagcccaacaccctggtgctgcccaaacacgccgacgccgacaacatcctcgtgatccag
cagggccaggccaccgtgacagtggccaacggcaacaacagaaagagcttcaacctggacgagggccacgccctgagaat
ccccagcggcttcatcagctacatcctgaacagacacgacaatcagaacctgagggtggccaagatcagcatgcccgtgaaca
cccctggccagttcgaggacttcttccccgcatcctcccgggaccagagcagctacctgcagggcttcagccggaataccctg
gaagccgccttcaacgccgagttcaacgagatcagacgggtgctgctggaagagaacgctggcggagagcaggaagaacg
gggccagagaagatggtccaccagaagcagcgagaacaacgagggcgtgatcgtgaaggtgtccaaagaacacgtggaag
aactgaccaagcacgccaagagcgtgtccaagaagggctccgaggaagagggggacatcaccaaccccatcaatctgagag
agggcgagcccgacctgagcaacaacttcggcaagctgttcgaagtgaagcccgacaagaagaacccccagctgcaggacc
tggacatgatgctgacctgcgtggaaatcaaagagggggccctgatgctgccacacttcaactccaaagccatggtcatcgtgg
tcgtgaacaagggcaccggcaacctggaactggtggccgtgcggaaagagcagcagcagagaggccgcagagaggaaga
agaggacgaggacgaagaagaagagggatccaaccgggaagtgcggcggtacaccgccagactgaaagaaggcgacgtg
ttcatcatgcctgccgcccaccccgtggccatcaatgcctctagcgagctgcatctgctgggcttcggcattaacgccgagaaca
atcaccggatctttctggccggcgacaaagacaacgtgatcgaccagatcgagaagcaggccaaggacctggcctttcccggc
tctggcgaacaagtggaaaagctgatcaagaaccagaaagaaagccacttcgtgtccgccagaccccagagccagtctcaga
gccctagctcccccgagaaagagtctcctgagaaagaggaccaggaagaggaaaaccagggcggcaagggccctctgctg
agcatcctgaaggccttcaat
SEQ ID NO: 16 - AraH2 Nucleotide sequence
aggcagcagtgggaactgcagggcgacagaagatgccagtcccagctggaacgggccaacctgaggccttgcgagcagca
cctgatgcagaaaatccagcgcgacgaggacagctacggccgggatccttacagccccagccaggacccttactcccctagc
caggatcccgacagaagggacccctacagccctagcccctacgatagaagaggcgccggaagcagccagcaccaggaaag
atgctgcaacgagctgaacgagtttgagaacaaccagcgctgcatgtgcgaggccctgcagcagatcatggaaaatcagagc
gaccggctgcagggacggcagcaggaacagcagttcaagagagagctgcggaacctgccccagcagtgtggactgagagc
cccccagagatgcgacctggaagtggaaagcggcggcagagataggtac
SEQ ID NO: 17 - AraH3 Nucleotide sequence
gtgaccttcagacagggcggagaagagaatgagtgccagtttcagcggctgaacgcccagaggcccgacaacagaatcgag
agcgagggcggctacatcgagacatggaaccccaacaaccaggaatttcagtgcgctggggtggccctgagcaggaccgtg
ctgagaagaaatgccctgaggcggcccttctacagcaacgcccccctggaaatctacgtgcagcagggcagcggctacttcg
gcctgatctttcccggatgcccctccacctatgaggaacccgctcaggaaggcagacggtatcagagccagaagcctagcaga
cggttccaagtgggccaggacgatcccagccaacagcagcaggactctcaccagaaggtgcaccgcttcgacgagggcgac
ctgatcgctgtgccaaccggcgtggccttctggatgtacaacgacgaggataccgacgtcgtgaccgtgaccctgagcgacac
cagctccatccacaaccagctggaccagttccccaggcggttttacctggccggcaatcaggaacaggaatttctgagatacca
gcagcagcagggctccagaccccactacagacagatcagccctagagtgcggggcgacgaacaggaaaatgagggcagca
acatcttctccggctttgcccaggaatttctgcagcacgccttccaggtggaccggcagaccgtggaaaacctgagaggcgaga
acgagagagaggaacagggcgccatcgtgactgtgaagggcggcctgaggatcctgagccccgacgaagaggatgagtcc
tctagaagcccccccaaccgccgggaagagttcgatgaggaccgcagcagacctcagcagcgggggaagtacgacgagaa
caggcggggctacaagaacggcatcgaggaaacaatctgcagcgccagcgtgaagaagaatctgggccggtccagcaacc
ccgacatctacaatccacaggccggcagcctgcggagcgtgaacgaactggatctgcccatcctgggatggctgggcctgtct
gcccagcacggcaccatctaccggaacgccatgttcgtgcctcactacaccctgaatgcccacaccatcgtggtggctctgaac
ggccgcgcccacgtccaagtggtggacagcaacggcaatcgggtgtacgatgaagaactgcaggaaggacacgtcctggtg
gtgccccagattttgccgtggccgccaaggcccagtccgagaactatgagtatctggccttcaagaccgacagccggccctct
atcgccaatcaagccggcgagaacagcatcatcgacaacctgcccgaggaagtggtggccaacagctaccggctgcctaga
gagcaggcccggcagctgaagaacaacaaccctttcaagttcttcgtgcccccattcgaccaccagagcatgagagaggtggc
c
SEQ ID NO: 18 - AraH1-LAMP Nucleotide sequence
SIGNAL: (1) . . . (86)
STABILIZING: (87) . . . (1146)
AraH1: (1147) . . . (2943)
TM/CYTO: (2943) . . . (3066)
atggcgccccgcagcgcccggcgacccctgctgctgctactgctgttgctgctgctcggcctcatgcattgtgcgtcagcagca
atgtttatggtgaaaaatggcaacgggaccgcgtgcataatggccaacttctctgctgccttctcagtgaactacgacaccaaga
gtggccctaagaacatgacccttgacctgccatcagatgccacagtggtgctcaaccgcagctcctgtggaaaagagaacactt
ctgaccccagtctcgtgattgcttttggaagaggacatacactcactctcaatttcacgagaaatgcaacacgttacagcgttcagc
tcatgagttttgtttataacttgtcagacacacaccttttccccaatgcgagctccaaagaaatcaagactgtggaatctataactgac
atcagggcagatatagataaaaaatacagatgtgttagtggcacccaggtccacatgaacaacgtgaccgtaacgctccatgat
gccaccatccaggcgtacctttccaacagcagcttcagcaggggagagacacgctgtgaacaagacaggccttccccaacca
cagcgccccctgcgccacccagcccctcgccctcacccgtgcccaagagcccctctgtggacaagtacaacgtgagcggcac
caacgggacctgcctgctggccagcatggggctgcagctgaacctcacctatgagaggaaggacaacacgacggtgacaag
gcttctcaacatcaaccccaacaagacctcggccagcgggagctgcggcgcccacctggtgactctggagctgcacagcgag
ggcaccaccgtcctgctcttccagttcgggatgaatgcaagttctagccggtttttcctacaggaatccagttgaatacaattcttc
ctgacgccagagaccctgcctttaaagctgccaacggctccctgcgagcgctgcaggccacagtcggcaattcctacaagtgc
aacgcggaggagcacgtccgtgtcacgaaggcgttttcagtcaatatattcaaagtgtgggtccaggctttcaaggtggaaggtg
gccagtttggctctgtggaggagtgtctgctggacgagaacagcctcgagaagtccagcccctaccagaagaaaaccgagaa
cccctgcgcccagcggtgcctgcagtcttgtcagcaggaacccgacgacctgaagcagaaggcctgcgagagccggtgcac
caagctggaatacgaccccagatgcgtgtacgaccctagaggccacaccggcaccaccaaccagagaagccctccaggcga
gcggaccagaggcagacagcctggcgactacgacgacgacagacggcagcccagaagagaagagggcggcagatgggg
acctgccggccctagagagagagaacgcgaggaagattggagacagcccagagaggactggcggaggccttctcaccagc
agccccggaagatcagacccgagggcagagaaggcgagcaggaatggggcacacctggctctcacgtgcgcgaggaaac
cagccggaacaaccccttctacttcccctcccggcggttcagcaccagatacggcaaccagaacggccggatcagagtgctgc
agagattcgaccagcggagccggcagttccagaacctgcagaaccaccggatcgtgcagatcgaggccaagcccaacacc
ctggtgctgcccaaacacgccgacgccgacaacatcctcgtgatccagcagggccaggccaccgtgacagtggccaacggc
aacaacagaaagagcttcaacctggacgagggccacgccctgagaatccccagcggcttcatcagctacatcctgaacagac
acgacaatcagaacctgagggtggccaagatcagcatgcccgtgaacacccctggccagttcgaggacttcttccccgcatcct
cccgggaccagagcagctacctgcagggcttcagccggaataccctggaagccgccttcaacgccgagttcaacgagatcag
acgggtgctgctggaagagaacgctggcggagagcaggaagaacggggccagagaagatggtccaccagaagcagcgag
aacaacgagggcgtgatcgtgaaggtgtccaaagaacacgtggaagaactgaccaagcacgccaagagcgtgtccaagaag
ggctccgaggaagagggggacatcaccaaccccatcaatctgagagagggcgagcccgacctgagcaacaacttcggcaa
gctgttcgaagtgaagcccgacaagaagaacccccagctgcaggacctggacatgatgctgacctgcgtggaaatcaaagag
ggggccctgatgctgccacacttcaactccaaagccatggtcatcgtggtcgtgaacaagggcaccggcaacctggaactggt
ggccgtgcggaaagagcagcagcagagaggccgcagagaggaagaagaggacgaggacgaagaagaagagggatcca
accgggaagtgcggcggtacaccgccagactgaaagaaggcgacgtgttcatcatgcctgccgcccaccccgtggccatcaa
tgcctctagcgagctgcatctgctgggcttcggcattaacgccgagaacaatcaccggatctttctggccggcgacaaagacaa
cgtgatcgaccagatcgagaagcaggccaaggacctggcctttcccggctctggcgaacaagtggaaaagctgatcaagaac
cagaaagaaagccacttcgtgtccgccagaccccagagccagtctcagagccctagctcccccgagaaagagtctcctgaga
aagaggaccaggaagaggaaaaccagggcggcaagggccctctgctgagcatcctgaaggccttcaatgaattcacgctgat
ccccatcgctgtgggtggtgccctggcggggctggtcctcatcgtcctcatcgcctacctcgtcggcaggaagaggagtcacg
caggctaccagactatctag
SEQ ID NO: 19 - AraH2-LAMP Nucleotide sequence
SIGNAL: (1) . . . (86)
STABILIZING: (87) . . . (1146)
ARA H2: (1147)-(1600)
TM/CYTO: (1601) . . . (1716)
atggcgccccgcagcgcccggcgacccctgctgctgctactgctgttgctgctgctcggcctcatgcattgtgcgtcagcagca
atgtttatggtgaaaaatggcaacgggaccgcgtgcataatggccaacttctctgctgccttctcagtgaactacgacaccaaga
gtggccctaagaacatgacccttgacctgccatcagatgccacagtggtgctcaaccgcagctcctgtggaaaagagaacactt
ctgaccccagtctcgtgattgcttttggaagaggacatacactcactctcaatttcacgagaaatgcaacacgttacagcgttcagc
tcatgagttttgtttataacttgtcagacacacaccttttccccaatgcgagctccaaagaaatcaagactgtggaatctataactgac
atcagggcagatatagataaaaaatacagatgtgttagtggcacccaggtccacatgaacaacgtgaccgtaacgctccatgat
gccaccatccaggcgtacctttccaacagcagcttcagcaggggagagacacgctgtgaacaagacaggccttccccaacca
cagcgccccctgcgccacccagcccctcgccctcacccgtgcccaagagcccctctgtggacaagtacaacgtgagcggcac
caacgggacctgcctgctggccagcatggggctgcagctgaacctcacctatgagaggaaggacaacacgacggtgacaag
gcttctcaacatcaaccccaacaagacctcggccagcgggagctgcggcgcccacctggtgactctggagctgcacagcgag
ggcaccaccgtcctgctcttccagttcgggatgaatgcaagttctagccggtttttcctacaaggaatccagttgaatacaattcttc
ctgacgccagagaccctgcctttaaagctgccaacggctccctgcgagcgctgcaggccacagtcggcaattcctacaagtgc
aacgcggaggagcacgtccgtgtcacgaaggcgttttcagtcaatatattcaaagtgtgggtccaggctttcaaggtggaaggtg
gccagtttggctctgtggaggagtgtctgctggacgagaacagcctcgagaggcagcagtgggaactgcagggcgacagaa
gatgccagtcccagctggaacgggccaacctgaggccttgcgagcagcacctgatgcagaaaatccagcgcgacgaggaca
gctacggccgggatccttacagccccagccaggacccttactcccctagccaggatcccgacagaagggacccctacagccc
tagcccctacgatagaagaggcgccggaagcagccagcaccaggaaagatgctgcaacgagctgaacgagtttgagaacaa
ccagcgctgcatgtgcgaggccctgcagcagatcatggaaaatcagagcgaccggctgcagggacggcagcaggaacag
cagttcaagagagagctgcggaacctgccccagcagtgtggactaagagccccccagagatgcgacctggaagtggaaagc
ggcggcagagataggtacgaattcacgctgatccccatcgctgtgggtggtgccctggcggggctggtcctcatcgtcctcatc
gcctacctcgtcggcaggaagaggagtcacgcaggctaccagactatctag
SEQ ID NO: 20 - AraH3-LAMP Nucleotide sequence
SIGNAL: (1) . . . (86)
STABILIZING: (87) . . . (1146)
AraH3: (1147) . . . (2623)
TM/CYTO: (2624) . . . (2739)
atggcgccccgcagcgcccggcgacccctgctgctgctactgctgttgctgctgctcggcctcatgcattgtgcgtcagcagca
atgtttatggtgaaaaatggcaacgggaccgcgtgcataatggccaacttctctgctgccttctcagtgaactacgacaccaaga
gtggccctaagaacatgacccttgacctgccatcagatgccacagtggtgctcaaccgcagctcctgtggaaaagagaacactt
ctgaccccagtctcgtgattgcttttggaagaggacatacactcactctcaatttcacgagaaatgcaacacgttacagcgttcagc
tcatgagttttgtttataacttgtcagacacacaccttttccccaatgcgagctccaaagaaatcaagactgtggaatctataactgac
atcagggcagatatagataaaaaatacagatgtgttagtggcacccaggtccacatgaacaacgtgaccgtaacgctccatgat
gccaccatccaggcgtacctttccaacagcagcttcagcaggggagagacacgctgtgaacaagacaggccttccccaacca
cagcgccccctgcgccacccagcccctcgccctcacccgtgcccaagagcccctctgtggacaagtacaacgtgagcggcac
caacgggacctgcctgctggccagcatggggctgcagctgaacctcacctatgagaggaaggacaacacgacggtgacaag
gcttctcaacatcaaccccaacaagacctcggccagcgggagctgcggcgcccacctggtgactctggagctgcacagcgag
ggcaccaccgtcctgctcttccagttcgggatgaatgcaagttctagccggtttttcctacaaggaatccagttgaatacaattcttc
ctgacgccagagaccctgcctttaaagctgccaacggctccctgcgagcgctgcaggccacagtcggcaattcctacaagtgc
aacgcggaggagcacgtccgtgtcacgaaggcgttttcagtcaatatattcaaagtgtgggtccaggctttcaaggtggaaggtg
gccagtttggctctgtggaggagtgtctgctggacgagaacagcctcgaggtgaccttcagacagggcggagaagagaatga
gtgccagtttcagcggctgaacgcccagaggcccgacaacagaatcgagagcgagggcggctacatcgagacatggaaccc
caacaaccaggaatttcagtgcgctggggtggccctgagcaggaccgtgctgagaagaaatgccctgaggcggcccttctaca
gcaacgcccccctggaaatctacgtgcagcagggcagcggctacttcggcctgatctttcccggatgcccctccacctatgagg
aacccgctcaggaaggcagacggtatcagagccagaagcctagcagacggttccaagtgggccaggacgatcccagccaac
agcagcaggactctcaccagaaggtgcaccgcttcgacgagggcgacctgatcgctgtgccaaccggcgtggccttctggat
gtacaacgacgaggataccgacgtcgtgaccgtgaccctgagcgacaccagctccatccacaaccagctggaccagttcccc
aggcggttttacctggccggcaatcaggaacaggaatttctgagataccagcagcagcagggctccagaccccactacagaca
gatcagccctagagtgcggggcgacgaacaggaaaatgagggcagcaacatcttctccggctttgcccaggaatttctgcagc
acgccttccaggtggaccggcagaccgtggaaaacctgagaggcgagaacgagagagaggaacagggcgccatcgtgact
gtgaagggcggcctgaggatcctgagccccgacgaagaggatgagtcctctagaagcccccccaaccgccgggaagagttc
gatgaggaccgcagcagacctcagcagcgggggaagtacgacgagaacaggcggggctacaagaacggcatcgaggaaa
caatctgcagcgccagcgtgaagaagaatctgggccggtccagcaaccccgacatctacaatccacaggccggcagcctgcg
gagcgtgaacgaactggatctgcccatcctgggatggctgggcctgtctgcccagcacggcaccatctaccggaacgccatgt
tcgtgcctcactacaccctgaatgcccacaccatcgtggtggctctgaacggccgcgcccacgtccaagtggtggacagcaac
ggcaatcgggtgtacgatgaagaactgcaggaaggacacgtcctggtggtgccccagaattttgccgtggccgccaaggccc
agtccgagaactatgagtatctggccttcaagaccgacagccggccctctatcgccaatcaagccggcgagaacagcatcatc
gacaacctgcccgaggaagtggtggccaacagctaccggctgcctagagagcaggcccggcagctgaagaacaacaaccct
ttcaagttcttcgtgcccccattcgaccaccagagcatgagagaggtggccgaattcacgctgatccccatcgctgtgggtggtg
ccctggcggggctggtcctcatcgtcctcatcgcctacctcgtcggcaggaagaggagtcacgcaggctaccagactatctag
SEQ ID NO: 21 - AraH1-AraH2-AraH3-LAMP (AraH-LAMP, AraH1-H2-H3-LAMP)
Nucleotide sequence
SIGNAL: (1) . . . (86)
STABILIZING: (87) . . . (1146)
AraH1: (1147) . . . (2949)
LINKER: (2950) . . . (2961)
AraH2: (2962) . . . (3414)
LINKER: (3415) . . . (3426)
AraH3: (3427) . . . (4902)
TM/CYTO: (4903) . . . (5019)
atggcgccccgcagcgcccggcgacccctgctgctgctactgctgttgctgctgctcggcctcatgcattgtgcgtcagcagca
atgtttatggtgaaaaatggcaacgggaccgcgtgcataatggccaacttctctgctgccttctcagtgaactacgacaccaaga
gtggccctaagaacatgacccttgacctgccatcagatgccacagtggtgctcaaccgcagctcctgtggaaaagagaacactt
ctgaccccagtctcgtgattgcttttggaagaggacatacactcactctcaatttcacgagaaatgcaacacgttacagcgttcagc
tcatgagttttgtttataacttgtcagacacacaccttttccccaatgcgagctccaaagaaatcaagactgtggaatctataactgac
atcagggcagatatagataaaaaatacagatgtgttagtggcacccaggtccacatgaacaacgtgaccgtaacgctccatgat
gccaccatccaggcgtacctttccaacagcagcttcagcaggggagagacacgctgtgaacaagacaggccttccccaacca
cagcgccccctgcgccacccagcccctcgccctcacccgtgcccaagagcccctctgtggacaagtacaacgtgagcggcac
caacgggacctgcctgctggccagcatggggctgcagctgaacctcacctatgagaggaaggacaacacgacggtgacaag
gcttctcaacatcaaccccaacaagacctcggccagcgggagctgcggcgcccacctggtgactctggagctgcacagcgag
ggcaccaccgtcctgctcttccagttcgggatgaatgcaagttctagccggtttttcctacaaggaatccagttgaatacaattcttc
ctgacgccagagaccctgcctttaaagctgccaacggctccctgcgagcgctgcaggccacagtcggcaattcctacaagtgc
aacgcggaggagcacgtccgtgtcacgaaggcgttttcagtcaatatattcaaagtgtgggtccaggctttcaaggtggaaggtg
gccagtttggctctgtggaggagtgtctgctggacgagaacagcctcgagaagtccagcccctaccagaagaaaaccgagaa
cccctgcgcccagcggtgcctgcagtcttgtcagcaggaacccgacgacctgaagcagaaggcctgcgagagccggtgcac
caagctggaatacgaccccagatgcgtgtacgaccctagaggccacaccggcaccaccaaccagagaagccctccaggcga
gcggaccagaggcagacagcctggcgactacgacgacgacagacggcagcccagaagagaagagggcggcagatgggg
acctgccggccctagagagagagaacgcgaggaagattggagacagcccagagaggactggcggaggccttctcaccagc
agccccggaagatcagacccgagggcagagaaggcgagcaggaatggggcacacctggctctcacgtgcgcgaggaaac
cagccggaacaaccccttctacttcccctccccggcggttcagcaccagatacggcaaccagaacggccggatcagagtgctgc
agagattcgaccagcggagccggcagttccagaacctgcagaaccaccggatcgtgcagatcgaggccaagcccaacacc
ctggtgctgcccaaacacgccgacgccgacaacatcctcgtgatccagcagggccaggccaccgtgacagtggccaacggc
aacaacagaaagagcttcaacctggacgagggccacgccctgagaatccccagcggcttcatcagctacatcctgaacagac
acgacaatcagaacctgagggtggccaagatcagcatgcccgtgaacacccctggccagttcgaggacttcttccccgcatcct
cccgggaccagagcagctacctgcagggcttcagccggaataccctggaagccgccttcaacgccgagttcaacgagatcag
acgggtgctgctggaagagaacgctggcggagagcaggaagaacggggccagagaagatggtccaccagaagcagcgag
aacaacgagggcgtgatcgtgaaggtgtccaaagaacacgtggaagaactgaccaagcacgccaagagcgtgtccaagaag
ggctccgaggaagagggggacatcaccaaccccatcaatctgagagagggcgagcccgacctgagcaacaacttcggcaa
gctgttcgaagtgaagcccgacaagaagaacccccagctgcaggacctggacatgatgctgacctgcgtggaaatcaaagag
ggggccctgatgctgccacacttcaactccaaagccatggtcatcgtggtcgtgaacaagggcaccggcaacctggaactggt
ggccgtgcggaaagagcagcagcagagaggccgcagagaggaagaagaggacgaggacgaagaagaagagggatcca
accgggaagtgcggcggtacaccgccagactgaaagaaggcgacgtgttcatcatgcctgccgcccaccccgtggccatcaa
tgcctctagcgagctgcatctgagggcttcggcattaacgccgagaacaatcaccggatctttctggccggcgacaaagacaa
cgtgatcgaccagatcgagaagcaggccaaggacctggcctttcccggctctggcgaacaagtggaaaagctgatcaagaac
cagaaagaaagccacttcgtgtccgccagaccccagagccagtctcagagccctagctcccccgagaaagagtctcctgag
aaagaggaccaggaagaggaaaaccagggcggcaagggccctctgctgagcatcctgaaggccttcaatggcggcggagg
caggcagcagtgggaactgcagggcgacagaagatgccagtcccagctggaacgggccaacctgaggccttgcgagcagc
acctgatgcagaaaatccagcgcgacgaggacagctacggccgggatccttacagccccagccaggacccttactcccct
agccaggatcccgacagaagggacccctacagccctagcccctacgatagaagaggcgccggaagcagccagcaccagga
aagatgctgcaacgagctgaacgagtttgagaacaaccagcgctgcatgtgcgaggccctgcagcagatcatggaaaatcag
agcgaccggctgcagggacggcagcaggaacagcagttcaagagagagctgcggaacctgccccagcagtgtggactg
agagccccccagagatgcgacctggaagtggaaagcggcggcagagatcggtacggcggagggggcgtgaccttcagaca
gggcggagaagagaatgagtgccagtttcagcggctgaacgcccagaggcccgacaacagaatcgagagcgagggcggct
acatcgagacatggaaccccaacaaccaggaatttcagtgcgctggggtggccctgagcaggaccgtgctgagaagaaat
gccctgaggcggcccttctacagcaacgcccccctggaaatctacgtgcagcagggcagcggctacttcggcctgatctttccc
ggatgcccctccacctatgaggaacccgctcaggaaggcagacggtatcagagccagaagcctagcagacggttccaagtgg
gccaggacgatcccagccaacagcagcaggactctcaccagaaggtgcaccgcttcgacgagggcgacctgatcgctgtgc
caaccggcgtggccttctggatgtacaacgacgaggataccgacgtcgtgaccgtgaccctgagcgacaccagctccatccac
aaccagctggaccagttccccaggcggttttacctggccggcaatcaggaacaggaatttctgagataccagcagcagcaggg
ctccagaccccactacagacagatcagccctagagtgcggggcgacgaacaggaaaatgagggcagcaacatcttctccggc
tttgcccaggaatttctgcagcacgccttccaggtggaccggcagaccgtggaaaacctgagaggcgagaacgagagagagg
aacagggcgccatcgtgactgtgaagggcggcctgaggatcctgagccccgacgaagaggatgagtcctctagaagccccc
ccaaccgccgggaagagttcgatgaggaccgcagcagacctcagcagcgggggaagtacgacgagaacaggcggggcta
caagaacggcatcgaggaaacaatctgcagcgccagcgtgaagaagaatctgggccggtccagcaaccccgacatctacaat
ccacaggccggcagcctgcggagcgtgaacgaactggatctgcccatcctgggatggctgggcctgtctgcccagcacggc
accatctaccggaacgccatgttcgtgcctcactacaccctgaatgcccacaccatcgtggtggctctgaacggccgcgcccac
gtccaagtggtggacagcaacggcaatcgggtgtacgatgaagaactgcaggaaggacacgtcctggtggtgccccagaattt
tgccgtggccgccaaggcccagtccgagaactatgagtatctggccttcaagaccgacagccggccctctatcgccaatcaag
ccggcgagaacagcatcatcgacaacctgcccgaggaagtggtggccaacagctaccggctgcctagagagcaggcccgg
cagctgaagaacaacaaccctttcaagttcttcgtgcccccattcgaccaccagagcatgagagaggtggccgaattcacgctg
atccccatcgctgtgggtggtgccctggcggggctggtcctcatcgtcctcatcgcctacctcgtcggcaggaagaggagtcac
gcaggctaccagactatctag
SEQ ID NO: 22 - LAMP-3 (DC-LAMP) amino acid sequence
SIGNAL: (1) . . . (27)
STABILIZING: (28) . . . (381)
TM/CYTO: (382) . . . (416)
MPRQLSAAAALFASLAVILHDGSQMRAKAFPETRDYSQPTAAATVQDIKKPVQQ
PAKQAPHQTLAARFMDGHITFQTAATVKIPTTTPATTKNTATTSPITYTLVTTQAT
PNNSHTAPPVTEVTVGPSLAPYSLPPTITPPAHTTGTSSSTVSHTTGNTTQPSNQTT
LPATLSIALHKSTTGQKPVQPTHAPGTTAAAHNTTRTAAPASTVPGPTLAPQPSS
VKTGIYQVLNGSRLCIKAEMGIQLIVQDKESVFSPRRYFNIDPNATQASGNCGTR
KSNLLLNFQGGFVNLTFTKDEESYYISEVGAYLTVSDPETIYQGIKHAVVMFQTA
VGHSFKCVSEQSLQLSAHLQVKTTDVQLQAFDEEDDHFGNVDECSSDYTIVLPVI
GAIVVGLCLMGMGVYKIRLRCQSSGYQRI
SEQ ID NO: 23 - LAMP2 amino acid sequence
SIGNAL: (1) . . . (28)
STABILIZING: (29) . . . (375)
TM/CYTO: (376) . . . (408)
MVCFRLFPVPGSGLVLVCLVLGAVRSYALELNLTDSENATCLYAKWQMNFTVR
YETTNKTYKTVTISDHGTVTYNGSICGDDQNGPKIAVQFGPGFSWIANFTKAAST
YSIDSVSFSYNTGDNTTFPDAEDKGILTVDELLAIRIPLNDLFRCNSLSTLEKNDVV
QHYWDVLVQAFVQNGTVSTNEFLCDKDKTSTVAPTIHTTVPSPTTTPTPKEKPEA
GTYSVNNGNDTCLLATMGLQLNITQDKVASVININPNTTHSTGSCRSHTALLRLN
SSTIKYLDFVFAVKNENRFYLKEVNISMYLVNGSVFSIANNNLSYWMPPSSYMC
NKEQTVSVSGAFQINTFDLRVQPFNVTQGKYSTAQDCSADDDNFLVPIAVGAAL
AGVLILVLLAYFIGLKHHHAGYEQF
SEQ ID NO: 24 - LIMP II amino acid sequence
SIGNAL: (5) . . . (27)
STABILIZING: (28) . . . (433)
TM/CYTO: (434) . . . (478)
MGRCCFYTAGTLSLLLLVTSVTLLVARVFQKAVDQSIEKKIVLRNGTEAFDSWE
KPPLPVYTQFYFFNVTNPEEILRGETPRVEEVGPYTYRELRNKANIQFGDNGTTIS
AVSNKAYVFERDQSVGDPKIDLIRTLNIPVLTVIEWSQVHFLREIIEAMLKAYQQK
LFVTHTVDELLWGYKDEILSLIHVFRPDISPYFGLFYEKNGTNDGDYVFLTGEDS
YLNFTKIVEWNGKTSLDWWITDKCNMINGTDGDSFHPLITKDEVLYVFPSDFCRS
VYITFSDYESVQGLPAFRYKVPAEILANTSDNAGFCIPEGNCLGSGVLNVSICKNG
APIIMSFPHFYQADERFVSAIEGMHPNQEDHETFVDINPLTGIILKAAKRFQINIYV
KKLDDFVETGDIRTMVFPVMYLNESVHIDKETASRLKSMINTTLIITNIPYIIMALG
VFFGLVFTWLACKGQGSMDEGTADERAPLIRT
SEQ ID NO: 25 - ENDOLYN amino acid sequence
SIGNAL: (1) . . . (24)
STABILIZING: (25) . . . (162)
TM/CYTO: (163) . . . (197)
MSRLSRSLLWAATCLGVLCVLSADKNTTQHPNVTTLAPISNVTSAPVTSLPLVTT
PAPETCEGRNSCVSCFNVSVVNTTCFWIECKDESYCSHNSTVSDCQVGNTTDFCS
VSTATPVPTANSTAKPTVQPSPSTTSKTVTTSGTTNNTVTPTSQPVRKSTFDAASFI
GGIVLVLGVQAVIFFLYKFCKSKERNYHTL
SEQ ID NO: 26 - AraH3del nucleotide sequence
gtgaccttcagacagggcggagaagagaatgagtgccagtttcagcggctgaacgcccagaggcccgacaacagaatcgag
agcgagggcggctacatcgagacatggaaccccaacaaccaggaatttcagtgcgctggggtggccctgagcaggaccgtg
ctgagaagaaatgccctgaggcggcccttctacagcaacgcccccctggaaatctacgtgcagcagggcagcggctacttcg
gcctgatctttcccggatgcccctccacctatgaggaacccgctcaggaaggcagacggtatcagagccagaagcctagcaga
cggttccaagtgggccaggacgatcccagccaacagcagcaggactctcaccagaaggtgcaccgcttcgacgagggcgac
ctgatcgctgtgccaaccggcgtggccttctggatgtacaacgacgaggataccgacgtcgtgaccgtgaccctgagcgacac
cagctccatccacaaccagctggaccagttccccaggcggttttacctggccggcaatcaggaacaggaatttctgagatacca
gcagcagcagggctccagaccccactacagacagatcagccctagagtgcggggcgacgaacaggaaaatgagggcagca
acatcttctccggctttgcccaggaatttctgcagcacgccttccaggtggaccggcagaccgtggaaaacctgagaggcgaga
acgagagagaggaacagggcgccatcgtgactgtgaagggcggcctgaggatcctgagccccgacgaagaggatgagtcc
tctagaagcccccccaaccgccgggaagagttcgatgaggaccgcagcagacctcagcagcgggggaagtacgacgagaa
caggcggggctacaagaac
SEQ ID NO: 27 - AraH3del-LAMP nucleotide sequence
SIGNAL: (1) . . . (86)
STABILIZING: (87) . . . (1146)
ARAH3del: (1147) . . . (2064)
TM/CYTO: (2065) . . . (2181)
atggcgccccgcagcgcccggcgacccctgctgctgctactgctgttgctgctgctcggcctcatgcattgtgcgtcagcagca
atgtttatggtgaaaaatggcaacgggaccgcgtgcataatggccaacttctctgctgccttctcagtgaactacgacaccaaga
gtggccctaagaacatgacccttgacctgccatcagatgccacagtggtgctcaaccgcagctcctgtggaaaagagaacactt
ctgaccccagtctcgtgattgcttttggaagaggacatacactcactctcaatttcacgagaaatgcaacacgttacagcgtccag
ctcatgagttttgtttataacttgtcagacacacaccttttccccaatgcgagctccaaagaaatcaagactgtggaatctataactga
catcagggcagatatagataaaaaatacagatgtgttagtggcacccaggtccacatgaacaacgtgaccgtaacgctccatgat
gccaccatccaggcgtacctttccaacagcagcttcagccggggagagacacgctgtgaacaagacaggccttccccaacca
cagcgccccctgcgccacccagcccctcgccctcacccgtgcccaagagcccctctgtggacaagtacaacgtgagcggcac
caacgggacctgcctgctggccagcatggggctgcagctgaacctcacctatgagaggaaggacaacacgacggtgacaag
gcttctcaacatcaaccccaacaagacctcggccagcgggagctgcggcgcccacctggtgactctggagctgcacagcgag
ggcaccaccgtcctgctcttccagttcgggatgaatgcaagttctagccggtttttcctacaaggaatccagttgaatacaattcttc
ctgacgccagagaccctgcctttaaagctgccaacggctccctgcgagcgctgcaggccacagtcggcaattcctacaagtgc
aacgcggaggagcacgtccgtgtcacgaaggcgttttcagtcaatatattcaaagtgtgggtccaggctttcaaggtggaaggtg
gccagtttggctctgtggaggagtgtctgctggacgagaacagcctcgaggtgaccttcagacagggcggagaagagaatga
gtgccagtttcagcggctgaacgcccagaggcccgacaacagaatcgagagcgagggcggctacatcgagacatggaaccc
caacaaccaggaatttcagtgcgctggggtggccctgagcaggaccgtgctgagaagaaatgccctgaggcggcccttctaca
gcaacgcccccctggaaatctacgtgcagcagggcagcggctacttcggcctgatctttcccggatgcccctccacctatgagg
aacccgctcaggaaggcagacggtatcagagccagaagcctagcagacggttccaagtgggccaggacgatcccagccaac
agcagcaggactctcaccagaaggtgcaccgcttcgacgagggcgacctgatcgctgtgccaaccggcgtggccttctggat
gtacaacgacgaggataccgacgtcgtgaccgtgaccctgagcgacaccagctccatccacaaccagctggaccagttcccc
aggcggttttacctggccggcaatcaggaacaggaatttctgagataccagcagcagcagggctccagaccccactacagaca
gatcagccctagagtgcggggcgacgaacaggaaaatgagggcagcaacatcttctccggctttgcccaggaatttctgcagc
acgccttccaggtggaccggcagaccgtggaaaacctgagaggcgagaacgagagagaggaacagggcgccatcgtgact
gtgaagggcggcctgaggatcctgagccccgacgaagaggatgagtcctctagaagcccccccaaccgccgggaagagttc
gatgaggaccgcagcagacctcagcagcgggggaagtacgacgagaacaggcggggctacaagaacgaattcacgctgat
ccccatcgctgtgggtggtgccctggcggggctggtcctcatcgtcctcatcgcctacctcgtcggcaggaagaggagtcacg
caggctaccagactatctag
SEQ ID NO: 28 - AraH3-LAMP amino acid sequence
SIGNAL: (1) . . . (27)
STABILIZING: (28) . . . (380)
ARAH3: (381) . . . (884)
TM/CYTO: (885) . . . (912)
MAPRSARRPLLLLLLLLLLGLMHCASAAMFMVKNGNGTACIMANFSAAFSVNY
DTKSGPKNMTLDLPSDATVVLNRSSCGKENTSDPSLVIAFGRGHTLTLNFTRNAT
RYSVQLMSFVYNLSDTHLFPNASSKEIKTVESITDIRADIDKKYRCVSGTQVHMN
NVTVTLHDATIQAYLSNSSFSRGETRCEQDRPSPTTAPPAPPSPSPSPVPKSPSVDK
YNVSGTNGTCLLASMGLQLNLTYERKDNTTVTRLLNINPNKTSASGSCGAHLVT
LELHSEGTTVLLFQFGMNASSSRFFLQGIQLNTILPDARDPAFKAANGSLRALQA
TVGNSYKCNAEEHVRVTKAFSVNIFKVWVQAFKVEGGQFGSVEECLLDENSLEV
TFRQGGEENECQFQRLNAQRPDNRIESEGGYIETWNPNNQEFQCAGVALSRTVL
RRNALRRPFYSNAPLEIYVQQGSGYFGLIFPGCPSTYEEPAQEGRRYQSQKPSRRF
QVGQDDPSQQQQDSHQKVHRFDEGDLIAVPTGVAFWMYNDEDTDVVTVTLSD
TSSIHNQLDQFPRRFYLAGNQEQEFLRYQQQQGSRPHYRQISPRVRGDEQENEGS
NIFSGFAQEFLQHAFQVDRQTVENLRGENEREEQGAIVTVKGGLRILSPDEEDESS
RSPPNRREEFDEDRSRPQQRGKYDENRRGYKNGIEETICSASVKKNLGRSSNPDI
YNPQAGSLRSVNELDLPILGWLGLSAQHGTIYRNAMFVPHYTLNAHTIVVALNG
RAHVQVVDSNGNRVYDEELQEGHVLVVPQNFAVAAKAQSENYEYLAFKTDSRP
SIANQAGENSIIDNLPEEVVANSYRLPREQARQLKNNNPFKFFVPPFDHQSMREV
AEFTLIPIAVGGALAGLVLIVLIAYLVGRKRSHAGYQTI
SEQ ID NO: 29 - LAMP1 nucleotide sequence
SIGNAL: (1) . . . (481)
STABILIZING: (82) . . . (1140)
TM/CYTO: (1141) . . . (1251)
atggcgccccgcagcgcccggcgacccctgctgctgctactgctgttgctgctgctcggcctcatgcattgtgcgtcagcagca
atgtttatggtgaaaaatggcaacgggaccgcgtgcataatggccaacttctctgctgccttctcagtgaactacgacaccaaga
gtggccctaagaacatgacccttgacctgccatcagatgccacagtggtgctcaaccgcagctcctgtggaaaagagaacactt
ctgaccccagtctcgtgattgcttttggaagaggacatacactcactctcaatttcacgagaaatgcaacacgttacagcgtccag
ctcatgagttttgtttataacttgtcagacacacaccttttccccaatgcgagctccaaagaaatcaagactgtggaatctataactga
catcagggcagatatagataaaaaatacagatgtgttagtggcacccaggtccacatgaacaacgtgaccgtaacgctccatgat
gccaccatccaggcgtacctttccaacagcagcttcagccggggagagacacgctgtgaacaagacaggccttccccaacca
cagcgccccctgcgccacccagcccctcgccctcacccgtgcccaagagcccctctgtggacaagtacaacgtgagcggcac
caacgggacctgcctgctggccagcatggggctgcagctgaacctcacctatgagaggaaggacaacacgacggtgacaag
gcttctcaacatcaaccccaacaagacctcggccagcgggagctgcggcgcccacctggtgactctggagctgcacagcgag
ggcaccaccgtcctgctcttccagttcgggatgaatgcaagttctagccggtttttcctacaaggaatccagttgaatacaattcttc
ctgacgccagagaccctgcctttaaagctgccaacggctccctgcgagcgctgcaggccacagtcggcaattcctacaagtgc
aacgcggaggagcacgtccgtgtcacgaaggcgttttcagtcaatatattcaaagtgtgggtccaggctttcaaggtggaaggtg
gccagtttggctctgtggaggagtgtctgctggacgagaacagcacgctgatccccatcgctgtgggtggtgccctggcgggg
ctggtcctcatcgtcctcatcgcctacctcgtcggcaggaagaggagtcacgcaggctaccagactatctag
SEQ ID NO: 30 - LAMP1 amino acid sequence
SIGNAL: (1) . . . (27)
STABILIZING: (28) . . . (380)
TM/CYTO: (381) . . . (416)
MAPRSARRPLLLLLLLLLLGLMHCASAAMFMVKNGNGTACIMANFSAAFSVNY
DTKSGPKNMTLDLPSDATVVLNRSSCGKENTSDPSLVIAFGRGHTLTLNFTRNAT
RYSVQLMSFVYNLSDTHLFPNASSKEIKTVESITDIRADIDKKYRCVSGTQVHMN
NVTVTLHDATIQAYLSNSSFSRGETRCEQDRPSPTTAPPAPPSPSPSPVPKSPSVDK
YNVSGTNGTCLLASMGLQLNLTYERKDNTTVTRLLNINPNKTSASGSCGAHLVT
LELHSEGTTVLLFQFGMNASSSRFFLQGIQLNTILPDARDPAFKAANGSLRALQA
TVGNSYKCNAEEHVRVTKAFSVNIFKVWVQAFKVEGGQFGSVEECLLDENSTLI
PIAVGGNLAGLVLIVLIAYLVGRKRSHAGYQTI

1-50. (canceled)

51. An isolated or purified nucleic acid molecule comprising, in sequential order:
a nucleic acid sequence encoding a signal sequence;
a nucleic acid sequence encoding an intra-organelle stabilizing/trafficking domain;
a nucleic acid sequence encoding a peanut allergen domain, wherein the peanut allergen domain comprises at least one peanut allergen that does not include a native signal sequence for the peanut allergen;
a nucleic acid sequence encoding a transmembrane domain; and
a nucleic acid sequence encoding an endosomal/lysosomal targeting domain.

52. The nucleic acid molecule of claim 51, wherein the signal sequence, the intra-organelle stabilizing/trafficking domain, the transmembrane domain, and/or the endosomal/lysosomal targeting domain is derived from a lysosomal associated membrane protein (LAMP).

53. The nucleic acid molecule of claim 52, wherein LAMP is selected from LAMP1, LAMP2, LAMP-3 (DC-LAMP), LIMP II, or ENDOLYN.

54. The nucleic acid molecule of claim 51, wherein:
(a) the intra-organelle stabilizing/trafficking domain comprises amino acids 28 to 380 of SEQ ID NO: 1;
(b) the transmembrane domain comprises amino acids 1637 to 1660 of SEQ ID NO: 1 or the lumenal domain of LAMP; and/or
(c) the endosomal/lysosomal targeting domain comprises a YXXØ signal or the amino acid sequence LIRT.

55. The nucleic acid molecule of claim 54, wherein the YXXØ signal comprises the amino acid sequence YQTI, YQRI, YEQF, or YHTL.

56. The nucleic acid molecule of claim 51, wherein the nucleic acid sequence encoding a peanut allergen domain comprises a nucleic acid sequence that encodes two or more peanut allergenic epitopes.

57. The nucleic acid molecule of claim 56, wherein the nucleic acid sequence encoding a peanut allergen domain comprises a nucleic acid sequence that encodes three peanut allergens.

58. The nucleic acid molecule of claim 51, wherein the at least one peanut allergen comprises Ara H1, Ara H2, Ara H3, Ara H3del or a combination thereof.

59. The nucleic acid molecule of claim 51, wherein the at least one peanut allergen domain comprises the amino acid sequence of SEQ ID NO:2, SEQ ID NO:3, SEQ ID NO:4, and/or SEQ ID NO:5.

60. The nucleic acid molecule of claim 56 wherein the peanut allergenic epitopes or peanut allergens are separated by a linker.

61. The nucleic acid molecule of claim 60, wherein the linker comprises the amino acid sequence GGGG or GGGGS.

62. The nucleic acid molecule of claim 51, wherein said nucleic acid molecule is selected from:
(a) a nucleic acid molecule comprising a nucleic acid sequence encoding an amino acid sequence which is at least 70% identical to SEQ ID NO: 1;
(b) a nucleic acid molecule comprising a nucleic acid sequence encoding an amino acid sequence which is at least 80% identical to SEQ ID NO: 1;
(c) a nucleic acid molecule comprising a nucleic acid sequence encoding an amino acid sequence which is at least 90% identical to SEQ ID NO: 1;
(d) a nucleic acid molecule comprising a nucleic acid sequence encoding an amino acid sequence of SEQ ID NO: 1 or an amino acid sequence of SEQ ID NO: 1 in which one or 10 or less amino acids are substituted, deleted, inserted and/or added;
(e) a nucleic acid molecule comprising a nucleic acid sequence encoding an amino acid sequence of SEQ ID NO: 1; or
(f) a nucleic acid molecule comprising a nucleic acid sequence encoding amino acid sequence of SEQ ID NO:6, SEQ ID NO:7, SEQ ID NO:8 or SEQ ID NO:28.

63. The nucleic acid molecule of claim 51 wherein said nucleic acid molecule comprises deoxyribonucleic acid (DNA).

64. A vector comprising the nucleic acid molecule of claim 51.

65. A vector comprising the nucleic acid molecule of claim 62.

65. A cell comprising the nucleic acid molecule of claim 51.

66. A polypeptide encoded by the nucleic acid molecule of claim 51.

67. The nucleic acid molecule of claim 51 mixed with a pharmaceutically acceptable carrier.

68. The nucleic acid molecule of claim 62 mixed with a pharmaceutically acceptable carrier.

69. The vector of claim 64 mixed with a pharmaceutically acceptable carrier.

70. The vector of claim 65 mixed with a pharmaceutically acceptable carrier.

71. A method of preventing or treating a peanut allergic reaction in a subject in need thereof, comprising administering a therapeutically effective amount of the nucleic acid molecule of claim 51.

72. The method of claim 71, wherein the subject was exposed to a peanut allergen prior to the administering.

73. The method of claim 71, wherein the nucleic acid molecule is administered in an amount sufficient to:
(a) decrease the production of an IgE response;
(b) decrease plasma histidine levels;
(c) decrease production of IL-4;
(d) increase IFN-γ level;
(e) induce or increase the production of an allergen-specific IgG response; and/or
(f) attenuate an IgE response.

74. The method of claim 71, wherein the method reduces, eliminates, or prevents at least one clinical allergy symptom.

75. The method of 71, wherein the nucleic acid molecule is administered to the subject by intramuscular injection (IM) or intradermal (ID) injection.

76. The method of claim 71 wherein the subject is a human.
